(19) United States
(12) Patent Application Publication
(10) Pub. No.: US 2012/0232070 A1
(43) Pub. Date: Sep. 13, 2012

(54) Use of Indole Derivatives as Nurr-1 Activators for the Application Thereof as a Medicament for the Treatment of Parkinson's Disease

(75) Inventors: Jerome Amaudrut, Dijon (FR); Benaissa Boubia, Saint Apollinaire (FR); Maria Johanna Petronella Van Dongen, Hilversum (NL); Fabrice Guillier, Belleneuve (FR); Olivia Poupardin-Olivier, Varois Et Chaignot (FR)

(73) Assignee: Laboratoires Fournier SA, Dijon (FR)

(21) Appl. No.: 13/416,018

(22) Filed: Mar. 9, 2012

Related U.S. Application Data
(63) Continuation of application No. PCT/FR2010/051884, filed on Sep. 10, 2010.

(30) Foreign Application Priority Data
Sep. 11, 2009 (FR) 0956259
Jan. 8, 2010 (FR) 1050107

(51) Int. Cl. A61K 31/404; C07D 405/12; C07D 413/12; A61K 31/538; C07D 401/12; A61K 31/454; C07D 401/06; A61K 31/4439; C07D 417/06; C07D 409/06; A61K 31/5377; C07D 413/14; C07D 413/10; A61K 31/4245; A61P 25/28; A61P 25/16; C07D 409/12; C07D 403/12; A61K 31/4155; C07D 209/18

(52) U.S. Cl. 514/230.5; 548/509; 514/415; 548/454; 514/414; 544/105; 546/201; 514/323; 546/277.7; 514/339; 548/181; 548/468; 544/144; 514/235.2; 544/131; 548/132; 514/364; 548/144; 548/465; 548/364.7; 514/406

(57) ABSTRACT

Compounds derived from indole, notably useful in therapeutics, selected from:
i) the compounds of formula:

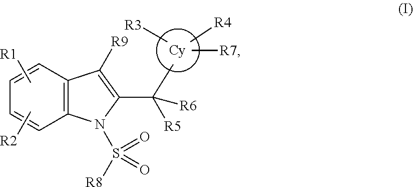

and
ii) the pharmaceutically acceptable salts of the compounds of formula (I);
   in which R1, R2, R3, R4, R5, R6, R8, R9 and Cy have defined meanings, and the use of such compounds in pharmaceuticals for the treatment of neurodegenerative diseases, particularly Parkinson's disease.

### CROSS REFERENCE TO RELATED APPLICATIONS

This application is a continuation of international application no. PCT/FR2010/051884, filed Sep. 10, 2010, designating the United States of America and published in French on Mar. 17, 2011 as WO 2011/030068, the entire disclosure of which is incorporated herein by reference. Priority is claimed based upon French patent application nos. FR 09 56259, filed Sep. 11, 2009, and FR 10 50107, filed Jan. 8, 2010, the entire disclosures of each of which are likewise incorporated herein by reference.

### BACKGROUND OF THE INVENTION

The present invention relates to new indole compounds; preferably derivatives of the indole benzoic type, as well as the method of preparation thereof and use thereof as the active principle of medicinal products, notably intended for the treatment and/or inhibition of diseases involving the NURR-1 nuclear receptors. More specifically, this invention relates to the use of these compounds for the manufacture of a medicinal product for the treatment and/or inhibition of neurodegenerative diseases and in particular Parkinson's disease.

Neurodegenerative diseases are defined as diseases characterized by progressive dysfunction of the nervous system. They are often associated with atrophy of the structures of the central or peripheral nervous system affected. They include, among others, diseases such as Alzheimer's disease, Creutzfeldt-Jakob disease, Huntington's disease, Parkinson's disease, lysosomal diseases, progressive supranuclear paralysis, multiple sclerosis and amyotrophic lateral sclerosis. Among these neurodegenerative diseases, Parkinson's disease is a disorder affecting about four million people world-wide. Although it affects people of any age, it is commoner in the elderly (with 2% of the population of people over 65 years affected by this disease). It is characterized by degeneration of the dopaminergic neurons of the substantia nigra. These types of neurons synthesize dopamine and use it as neurotransmitters.

It has been established that there is a relation between dopamine deficiency and nervous disorders. Dopamine plays a key role in the control of voluntary movements, cognitive functions and the development of behaviours associated with the emotions.

The existing therapeutic strategy for the treatment of Parkinson's disease is based on attenuation of the symptoms by making up for the dopamine deficiency by administering a metabolic precursor such as L-DOPA.

The current increase in frequency of this pathology has now made it necessary to develop new therapeutic agents, playing a beneficial role in survival and neuronal differentiation.

These developments led to the identification of compounds that are able to activate the nuclear receptors involved in the pathogenesis of Parkinson's disease.

Being strongly expressed in the brain, the NURR-1 transcription factor, a member of the superfamily of orphan nuclear receptors, has been identified as having an essential role in the development and maintenance of the dopaminergic neurons of the mesencephalon (Zetterstrom, Solomin et al. 1997, Science. 1997 Apr. 11; 276(5310):248-50).

The NURR-1 nuclear receptor is involved in maintenance of the dopaminergic phenotype via regulation of the specific genes of the dopaminergic neurons (DA). It also promotes survival of the DA neurons by protecting them from toxic aggression. The NURR-1 nuclear receptor therefore serves as a specific transcription factor of the dopaminergic neurons, for which the activities could be regulated by modulating dopaminergic neurotransmission in Parkinson's disease.

This receptor binds to DNA in the form of monomers, homodimers or heterodimers with RXR (Retinoid X Receptor), a nuclear receptor which is the heteropartner of many other members of the family of nuclear receptors. RXR takes part in numerous physiological processes such as lipid and glucose metabolism, development and differentiation. NURR-1 thus interacts with the α and γ isoforms of RXR. RXRα is expressed ubiquitously whereas expression of RXRγ is concentrated mainly in the brain and notably in the striatum, the hypothalamus and the hypophysis.

The NURR-1/RXRα and NURR-1/RXRγ complexes that are formed are capable of regulating transcription in response to a ligand of RXR. RXR therefore modulates the activation potential of transcription of NURR-1 positively.

Identification of compounds capable of inducing the activity of the NURR-1/RXRα and NURR-1/RXRγ complexes should accordingly offer novel routes for treating Parkinson's disease.

Heterocyclic active compounds for the treatment of Parkinson's disease are known from document WO2003/015780.

Moreover, documents WO2004/072050, FR 2 903 105, FR 2 903 106 and FR 2 903 107 describe compounds that are activators of the NURR-1 receptor, whereas the use of heterocyclic compounds that modulate the activity of the receptors of the NGFI-B family (of which NURR-1 is a member) is described in document WO2005/047268.

Furthermore, various indole compounds have been described in the prior art. Thus:
documents WO 00/46196 and WO 99/07678 disclose compounds that are derivatives of indole-2-carboxylic acid for their anti-inflammatory activity;
document WO 98/41092 describes derivatives of indole-2-carboxamide that are active against pain;
document WO2005/056522 describes derivatives of indole that find application as active principles of medicinal products for treating certain diseases of the cardiovascular system.

Finally, from the documents: Journal of Organic Chemistry, vol. 54, No. 14, 1989, pages 3264-3269; Journal of Organic Chemistry, American Chemical Society, Easton; vol. 57, 23 Oct. 1992, pages 5891-5899; Journal of Medicinal Chemistry, vol. 35, No. 26, 1992, pages 4854-4857; Journal of Chemical Society, Perkin Transactions 1, Chemical Society, No. 12, 1 Jan. 1991, pages 3165-3172; Journal of Organic Chemistry, American Chemical Society, Easton; vol. 50, No. 26, 27 Dec. 1985, pages 5451-5457; EP 1 086 950; Heterocycles, Elsevier Science Publishers B.V. Amsterdam, NL, vol. 34, No. 8, 27 Apr. 1996, pages 1613-1621 and WO2001082909, the following compounds are known:
2-[[1-(Phenylsulfonyl)-1H-indol-2-yl]carbonyl]-3-pyridinecarboxylic acid;
2-[[5-Methoxy-1-(phenyl sulfonyl)-1H-indol-2-yl]carbonyl]-3-pyridine-carboxylic acid;
2-[[6-Methoxy-1-(phenylsulfonyl)-1H-indol-2-yl]carbonyl]-3-pyridine-carboxylic acid;
4-[[1-(Phenylsulfonyl)-1H-indol-2-yl]carbonyl]-3-pyridinecarboxylic acid;
3-[[1-(Phenylsulfonyl)-1H-indol-2-yl]carbonyl]-4-pyridinecarboxylic acid;
4-[[5-Methoxy-1-(phenyl sulfonyl)-1H-indol-2-yl]carbonyl]-3-pyridine-carboxylic acid;
2-[[1-(Phenylsulfonyl)-1H-indol-2-yl]carbonyl]-benzoic acid;
3-[[5-Methoxy-1-(phenylsulfonyl)-1H-indol-2-yl]carbonyl]-4-pyridine-carboxylic acid;
4-[1-Hydroxy-1-[5-methoxy-1-(phenylsulfonyl)-1H-indol-2-yl]ethyl]-3-pyridinecarboxylic acid;
4-[1-[5-Methoxy-1-(phenylsulfonyl)-1H-indol-2-yl]ethyl]-3-pyridine-carboxylic acid;
4-[[3-Chloro-1-(phenylsulfonyl)-1H-indol-2-yl]carbonyl]-3-pyridine-carboxylic acid, methyl ester;
5-[Hydroxy[5-(methylthio)-1-(phenylsulfonyl)-1H-indol-2-yl]methyl]-2-furancarboxylic acid, ethyl ester;
5-[[5-(Methylthio)-1-(phenylsulfonyl)-1H-indol-2-yl]methyl]-2-furan-carboxylic acid, ethyl ester;
4-[[3-Bromo-1-(phenylsulfonyl)-1H-indol-2-yl]carbonyl]-3-pyridine-carboxylic acid;
4-[[1-(Phenylsulfonyl)-1H-inden-2-yl]carbonyl]-benzonitrile.
   In all these documents, said compounds are presented as synthesis intermediates.

### SUMMARY OF THE INVENTION

According to a first aspect, the present invention relates to compounds derived from indole which are NURR-1/RXRα and NURR-1/RXRγ agonists, capable of inhibiting the degeneration of neurons observed in Parkinson's disease for use thereof as a medicinal product and are selected from:

i) the compounds of formula:

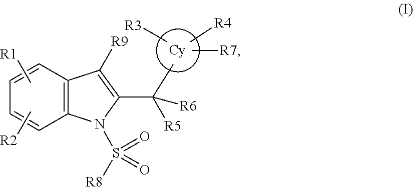

in which:
Cy represents a phenyl group or a heteroaromatic group having 5 or 6 ring members;
R1 and R2 each represent, independently of one another, a hydrogen atom, a halogen atom, a nitro group, an alkyl group having 1 to 4 carbon atoms, optionally fully or partially halogenated, an alkoxy group having 1 to 4 carbon atoms, a heterocyclic group having 4 to 6 atoms, a group —SCH₃, —OCF₃, —NH₂, —NHR, or —NR₂;
R3 and R4 each represent, independently of one another, a hydrogen atom, a halogen atom, an alkyl group having 1 to 4 carbon atoms, a hydroxyl group or an alkoxy group having 1 to 4 carbon atoms;
R5 and R6 each represent, independently of one another, a hydrogen atom, a halogen atom, an alkyl group having 1 to 4 carbon atoms, a hydroxyl group;
or R5 and R6 form, together with the carbon atom to which they are attached, a cycloalkyl group having 3 to 6 carbon atoms, an ethylene group (C═CH₂) or a carbonyl group (C═O);
R7 represents a —COOR group, a bioisosteric group of carboxylic acid or a —CN group;
R8 represents:
an alkyl group having 1 to 6 carbon atoms;
an aryl, heteroaryl, cyclic or heterocyclic group, which group may be unsubstituted or substituted with one, two or three substituents which may be identical or different, selected from halogen atoms, alkyl groups having 1 to 6 carbon atoms, optionally fully or partially halogenated, or optionally hydroxylated, alkoxy groups having 1 to 6 carbon atoms, optionally fully or partially halogenated, the phenoxy group, cyclic groups having 3 to 6 carbon atoms, aryl and heteroaryl groups, in particular phenyl and pyrazolyl, unsubstituted or substituted with one or two substituents, which may be identical or different, selected from halogen atoms and alkyl groups having 1 to 4 carbon atoms, the SCHF₂ and acyl-morpholine groups;
   R9 represents a hydrogen atom, a halogen atom or an alkyl group having 1 to 4 carbon atoms;
   R represents a hydrogen atom or an alkyl group having 1 to 4 carbon atoms.

ii) the pharmaceutically acceptable salts of said compounds of formula (I).

According to a second aspect, the invention relates to the aforementioned compounds for use as therapeutically active substances, in the treatment and/or inhibition of neurodegenerative diseases, in particular Parkinson's disease, as well as pharmaceutical compositions containing them.

According to a third aspect, the invention relates to the use of at least one compound of formula (I) or one of its pharmaceutically acceptable salts as an active principle for the preparation of a medicinal product intended for the treatment of diseases in which the NURR-1 receptor is involved, notably neurodegenerative diseases, such as in particular Parkinson's disease.

According to a fourth aspect, the present invention relates to novel compounds derived from indole which are NURR-1/RXRα and NURR-1/RXRγ agonists, capable of inhibiting the degeneration of neurons observed in Parkinson's disease, which are selected from the compounds of formula (I) as defined previously, excluding the following compounds:
2-[[1-(Phenylsulfonyl)-1H-indol-2-yl]carbonyl]-3-pyridinecarboxylic acid;
2-[[5-Methoxy-1-(phenylsulfonyl)-1H-indol-2-yl]carbonyl]-3-pyridine-carboxylic acid;
2-[[6-Methoxy-1-(phenylsulfonyl)-1H-indol-2-yl]carbonyl]-3-pyridine-carboxylic acid;
4-[[1-(Phenylsulfonyl)-1H-indol-2-yl]carbonyl]-3-pyridinecarboxylic acid;
3-[[1-(Phenylsulfonyl)-1H-indol-2-yl]carbonyl]-4-pyridinecarboxylic acid;
4-[[5-Methoxy-1-(phenylsulfonyl)-1H-indol-2-yl]carbonyl]-3-pyridine-carboxylic acid;
2-[[1-(Phenylsulfonyl)-1H-indol-2-yl]carbonyl]-benzoic acid;
3-[[5-Methoxy-1-(phenylsulfonyl)-1H-indol-2-yl]carbonyl]-4-pyridine-carboxylic acid;
4-[1-Hydroxy-1-[5-methoxy-1-(phenylsulfonyl)-1H-indol-2-yl]ethyl]-3-pyridinecarboxylic acid;
4-[1-[5-Methoxy-1-(phenylsulfonyl)-1H-indol-2-yl]ethyl]-3-pyridine-carboxylic acid;
4-[[3-Chloro-1-(phenylsulfonyl)-1H-indol-2-yl]carbonyl]-3-pyridine-carboxylic acid, methyl ester;
5-[Hydroxy[5-(methylthio)-1-(phenylsulfonyl)-1H-indol-2-yl]methyl]-2-furancarboxylic acid, ethyl ester;
5-[[5-(Methylthio)-1-(phenylsulfonyl)-1H-indol-2-yl]methyl]-2-furan-carboxylic acid, ethyl ester;
4-[[3-Bromo-1-(phenylsulfonyl)-1H-indol-2-yl]carbonyl]-3-pyridine-carboxylic acid;
4-[[1-(Phenylsulfonyl)-1H-inden-2-yl]carbonyl]-benzonitrile.

According to a last aspect of the invention, the present application aims to cover a method of inhibition and/or treatment of diseases in which the NURR-1 receptor is involved, notably neurodegenerative diseases, and more particularly Parkinson's disease, which consists of administering, to a patient in need thereof, a therapeutically effective amount of a compound of formula (I) or of a pharmaceutical composition containing said compound.

### BRIEF DESCRIPTION OF THE DRAWINGS

FIGS. 1 and 2 are graphs depicting the results of tests of experiments demonstrating the pharmacological activity of representative compounds according to the invention.

### DETAILED DESCRIPTION

“Alkyl group” means a saturated hydrocarbon chain, which can be linear and having at least 1 carbon atom or branched or cyclic and having at least 3 carbon atoms (the latter being also designated by the expression “cycloalkyl”). For example and without limitation, an alkyl group having from 1 to 6 carbon atoms can be a methyl, ethyl, propyl, butyl, pentyl, hexyl, 1-methylethyl, 1-methylpropyl, 2-methylpropyl, 1,1-dimethylethyl, 1-methylbutyl, 1,1-dimethylpropyl, 1-methylpentyl, 1,1-dimethylbutyl, cyclopropyl, cyclobutyl, cyclopentyl, cyclohexyl or cyclopentylmethyl group.

“Halogen” means a bromine, fluorine or chlorine atom.

“Partially or fully halogenated alkyl group” means an alkyl group as defined above in which one (or more) atom(s) of hydrogen is(are) replaced with a halogen atom or with halogen atoms. As an example of said group, we may mention the difluoromethyl or trifluoromethyl groups.

“Hydroxylated alkyl group” means an alkyl group as defined above in which a hydrogen atom is replaced by a hydroxyl group.

“Alkoxy group” means an OR group in which R is an alkyl group as defined previously. As an example of an alkoxy group having from 1 to 4 carbon atoms, we may mention the methoxy, ethoxy, propoxy, butoxy, 1-methylethoxy, 1,1-dimethylethoxy, 1-methylpropoxy, 2-methylpropoxy or cyclopropylmethoxy groups.

“Aryl group” means a monocyclic or bicyclic aromatic hydrocarbon group having from 6 to 12 carbon atoms. As an example of an aryl group, we may mention the phenyl and naphthyl groups.

“Heteroaryl group” means a monocyclic, bicyclic or tricyclic aromatic hydrocarbon group comprising at least one heteroatom in one of the cycles, said heteroatom being selected from nitrogen, oxygen and sulfur (as well as their oxidized forms, for example N-oxide, sulfoxide or sulfone).

A heteroaryl group can for example be a monocyclic group having 5 or 6 ring members, a bicyclic group having 7 to 11 ring members or a tricyclic group having 10 to 16 ring members, said group containing 1 to 3 heteroatoms, preferably 1 or 2 heteroatoms, selected from nitrogen, oxygen and sulfur.

As an example of a monocyclic heteroaryl group having 5 or 6 ring members (also denoted by the expression “heteroaromatic group”), we may mention the pyrrolyl, pyrazolyl, imidazolyl, oxazolyl, isoxazolyl, triazolyl, furanyl, thienyl, thiazolyl, isothiazolyl, thiadiazolyl, pyridyl, pyridazinyl, pyrimidinyl, pyrazinyl and triazinyl groups.

As an example of a bicyclic heteroaryl group, we may mention the benzothiazolyl, benzoxazolyl, benzoxazinone, benzoxadiazolyl, 1,3-benzodioxolyl, benzofuryl, benzopyrazinyl, benzothienyl, indolyl, indazolyl, benzimidazolyl, benzopyranyl, pyrrolopyridynyl, furopyridinyl, isoquinolinyl, quinolinyl and imidazothiazolyl groups.

“Cyclic group” means a saturated or partially unsaturated hydrocarbon group containing 1 to 3 rings having from 3 to 8 carbon atoms per ring.

As an example of a monocyclic group, we may mention the cyclopropyl, cyclobutyl, cyclopentyl, cyclohexyl, cycloheptyl, cyclobutenyl, cyclopentenyl and cyclohexenyl groups.

As an example of a bicyclic group, we may mention the 1,2,3,4-tetrahydronaphthalene group.

“Heterocyclic group” means a cyclic group as defined previously, of which one (or more) carbon atom(s) (optionally associated with one or more hydrogen atoms) is(are) replaced with one (or more) heteroatom(s) notably selected from oxygen and nitrogen.

As an example of a heterocyclic group, we may mention the monocyclic groups such as the tetrahydrofuryl, piperidinyl, pyrrolidinyl, tetrahydropyranyl, morpholinyl, thiamorpholinyl groups, or the bicyclic groups such as the dihydroquinasolinyl, dihydrobenzofuryl, notably 2,3-dihydrobenzofuryl, dihydrobenzothienyl, dihydrobenzoxazinyl, notably 3,4-dihydro-1,4-benzoxazinyl and 3-oxo-3,4-dihydro-1,4-benzoxazinyl, dihydrobenzodioxinyl, notably 2,3-dihydrobenzodioxinyl, dihydrobenzopyrannyl, 1,2,3,4-tetrahydroquinolinyl, 2,3-dihydroindolyl, dihydrobenzodioxepinyl, notably 3,4-dihydro-2H-1,5-benzodioxepinyl groups.

“Bioisosteric group of carboxylic acid” means a group displaying chemical and physical similarities and producing biological properties broadly similar to a carboxylic group as described in Lipinski, Annual Reports in Medicinal Chemistry, 1986, 21, p. 283 “Bioisosterism In Drug Design”; Graham, Theochem, 1995, 343, pp. 105-109 “Theoretical Studies Applied To Drug Design: ab initio Electronic Distributions In Bioisosteres”.

As an example of a bioisosteric group of carboxylic acid, we may mention the optionally substituted acylhydrazine, optionally substituted acylhydrazine carboxylate, optionally substituted alkyl and aryl sulfonylcarbamoyl, optionally substituted sulfonamide, oxadiazolone, optionally substituted phosphonate, optionally substituted isothiazole, optionally substituted isoxazole, optionally substituted isoxazolone tetrazole, optionally substituted thiazolidinedione, optionally substituted thioxothiazolidinone groups.

The compounds of formula (I) in which the substituents R5 and R6 are different possess a centre of asymmetry. For these compounds, the invention covers both the racemic compound and each of the optical isomers considered separately.

The compounds of formula (I) in which R7 represents a COOH group are carboxylic acids which can be used in the form of free acids or in the form of salts, said salts being obtained by combining the acid with a non-toxic mineral or organic base, preferably pharmaceutically acceptable. Among the mineral bases, it is possible for example to use the hydroxides of sodium, of potassium, of magnesium or of calcium. Among the organic bases, it is possible for example to use amines, amino alcohols, basic amino acids such as lysine or arginine or compounds bearing a quaternary ammonium function such as for example betaine or choline.

A first family of compounds according to the invention corresponds to formula I in which:

Cy represents a group of formula:

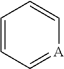

in which:
A represents a carbon atom monosubstituted with a hydrogen atom or a nitrogen atom;
or a heteroaromatic group having 5 ring members and having one or two heteroatoms;
R1 and R2 each represent, independently of one another, a hydrogen atom, a halogen atom, an alkyl group having 1 to 4 carbon atoms, optionally fully or partially halogenated, an alkoxy group having 1 to 4 carbon atoms, a heterocyclic group having 4 to 6 atoms or a group OCF₃;
R3 and R4 each represent, independently of one another, a hydrogen atom, a halogen atom, an alkyl group having 1 to 4 carbon atoms, a hydroxyl group or an alkoxy group having 1 to 4 carbon atoms;
R5 and R6 each represent, independently of one another, a hydrogen atom, a halogen atom, an alkyl group having 1 to 4 carbon atoms, a hydroxyl group;
or R5 and R6 form, with the carbon atom to which they are attached, an ethylene or carbonyl group;
R7 represents a —COOR group, a bioisosteric group of carboxylic acid or a —CN group;
R8 represents:
an alkyl group having 1 to 6 carbon atoms,
an aryl, heteroaryl, cyclic or heterocyclic group, which group may be unsubstituted or substituted with one, two or three substituents which may be identical or different, selected from halogen atoms, alkyl groups having 1 to 6 carbon atoms, optionally fully or partially halogenated, or optionally hydroxylated, alkoxy groups having 1 to 6 carbon atoms, optionally fully or partially halogenated, the phenoxy group, cyclic groups having 3 to 6 carbon atoms, aryl and heteroaryl groups, in particular phenyl and pyrazolyl, unsubstituted or substituted with one or two substituents, which may be identical or different, selected from halogen atoms and alkyl groups having 1 to 4 carbon atoms, the SCHF₂ and acyl-morpholine groups;
   R9 represents a hydrogen atom, a halogen atom or an alkyl group having 1 to 4 carbon atoms, R represents a hydrogen atom or an alkyl group (linear or branched) having 1 to 4 carbon atoms.

A preferred family of compounds according to the invention is constituted of the aforementioned compounds of formula I, in which:

R8 represents:
an alkyl group having 1 to 6 carbon atoms;
a phenyl group substituted with one or two substituents which may be identical or different, selected from halogen atoms, alkyl groups having 1 to 6 carbon atoms, optionally fully or partially halogenated, or optionally hydroxylated, alkoxy groups having 1 to 6 carbon atoms, optionally fully or partially halogenated, the phenoxy group, cyclic groups having 3 to 6 carbon atoms, aryl and heteroaryl groups, in particular phenyl and pyrazolyl, unsubstituted or substituted with one or two substituents, which may be identical or different, selected from halogen atoms and alkyl groups having 1 to 4 carbon atoms, the SCHF₂ and acyl-morpholine groups;
a naphthyl group; a thienyl group, unsubstituted or substituted with a phenyl group; a pyridinyl group unsubstituted or substituted with a substituent selected from alkoxy groups having 1 to 4 carbon atoms, the group phenoxy, heterocyclic groups having 6 ring members, in particular the morpholinyl group; a benzofuranyl group; a dihydrobenzoxazinone group substituted with a methyl group;
a tetrahydronaphthyl group, unsubstituted or substituted with one to four alkyl groups having 1 to 4 carbon atoms, a dihydrobenzodioxinyl group unsubstituted or substituted with an alkyl group having 1 to 4 carbon atoms, a dihydrobenzodioxazinyl group unsubstituted or substituted with an alkyl group having 1 to 4 carbon atoms, a dihydrobenzodioxepinyl group, a piperidinyl group, a dihydrobenzofuranyl group unsubstituted or substituted with one or two alkyl groups having 1 to 4 carbon atoms, a dihydrobenzopyranyl group unsubstituted or substituted with one or two alkyl groups having 1 to 4 carbon atoms.

Among the compounds of the invention, the compounds of formula I are more particularly preferred in which at least one of the following conditions is fulfilled:

Cy represents a phenyl, pyridyl, furanyl, thienyl, pyrrolyl or thiazolyl nucleus;
R1 represents a hydrogen atom, chlorine atom, bromine atom, a group —CF₃, OCH₃, —OCF₃, —C(CH₃)₃ or pyrrolidinyl;
R2 represents a hydrogen atom;
R3 represents the hydrogen atom, chlorine atom, fluorine atom, a hydroxyl group, a methyl group or a methoxy group;
R4 represents a hydrogen atom or fluorine atom;
R5 and R6 each represent, independently of one another, a hydrogen atom, a methyl or hydroxy group or form, together with the carbon atom to which they are attached, an ethylene or carbonyl group;
R8 represents a phenyl group substituted with a C₃-C₄ branched alkyl group; and
R9 represents a hydrogen atom, a fluorine atom or a methyl group, preferably a hydrogen atom.

Among the compounds of the invention, the compounds of formula I are further preferred in which the group R7 represents a bioisosteric group of carboxylic acid and more particularly the optionally substituted isoxazolone, oxadiazolone, optionally substituted alkyl and aryl sulfonylcarbamoyl groups.

As particularly preferred compounds, we may mention:
4-[[1-[[3-(1-methylethyl)phenyl]sulfonyl]-5-(trifluoromethyl)-1H-indol-2-yl]methyl]benzoic acid,
4-[[1-[[3-(1,1-dimethylethyl)phenyl]sulfonyl]-5-(trifluoromethyl)-1H-indol-2-yl]methyl]benzoic acid,
6-[[1-[[3-(1,1-dimethylethyl)phenyl]sulfonyl]-5-(trifluoromethyl)-1H-indol-2-yl]hydroxymethyl]-3-pyridinecarboxylic acid,
4-[[1-[[3-(1,1-dimethylethyl)phenyl]sulfonyl]-5-trifluoromethyl-1H-indol-2-yl]methyl]-3-fluoro-benzoic acid,
5-[[1-[[3-(1,1-dimethylethyl)-phenyl]sulfonyl]-5-trifluoromethyl-1H-indol-2-yl]methyl]-furan-2-carboxylic acid,
4-[[1-[[3-(1,1-dimethylethyl)-phenyl]sulfonyl]-5-trifluoromethyl-1H-indol-2-yl]methyl]-thiophene-2-carboxylic acid,
5-[[1-[[4-(1-methylethyl)-phenyl]sulfonyl]-5-trifluoromethyl-1H-indol-2-yl]methyl]-thiophene-2-carboxylic acid,
4-[[1-[[4-(1-methylethyl)-phenyl]sulfonyl]-5-trifluoromethyl-1H-indol-2-yl]methyl]-thiophene-2-carboxylic acid,
5-[[1-[[4-methyl-3,4-dihydro-2H-benzo[1,4]oxazin-6-yl]-sulfonyl]-5-trifluoromethyl-1H-indol-2-yl]methyl]-thiophene-2-carboxylic acid,
4-[[1-[(4-methyl-3,4-dihydro-2H-benzo[1,4]oxazin-6-yl)-sulfonyl]-5-trifluoromethyl-1H-indol-2-yl]methyl]-thiophene-2-carboxylic acid,
5-[[1-[(4-methyl-3,4-dihydro-2H-benzo[1,4]oxazin-6-yl)-sulfonyl]-5-trifluoromethyl-1H-indol-2-yl]methyl]-furan-2-carboxylic acid,
5-[[1-[[4-methyl-3,4-dihydro-2H-benzo[1,4]oxazin-6-yl)-sulfonyl]-5-trifluoromethyl-1H-indol-2-yl]methyl]-furan-3-carboxylic acid,
4-{[1-[[3-(1,1-dimethylethyl)-phenyl]sulfonyl]-5-trifluoromethyl-1H-indol-2-yl]-hydroxymethyl}-1-methyl-1H-pyrrol-2-yl-carboxylic acid (1,1-dimethyl-ethyl)ester,
2-[[1-[[3-(1,1-dimethylethyl)-phenyl]-sulfonyl]-3-methyl-5-trifluoro-methyl-1H-indol-2-yl]methyl]-thiazole-4-carboxylic acid, ethyl ester,
2-[[1-[[3-(1,1-dimethylethyl)-phenyl]sulfonyl]-5-trifluoromethyl-1H-indol-2-yl]methyl]-thiazole-4-carboxylic acid, ethyl ester,
4-[[1-[[3-(1,1-dimethylethyl)phenyl]sulfonyl]-5-(trifluoro)-6-fluoro-1H-indol-2-yl]methyl]benzoic acid, methyl ester,
4-[[1-[[3-(1-methylethyl)phenyl]sulfonyl]-5-(trifluoromethyl)-1H-indol-2-yl]methyl]benzoic acid,
4-[[1-[[3-(1,1-dimethylethyl)phenyl]sulfonyl]-3-fluoro-5-(trifluoromethyl)-1H-indol-2-yl]methyl]-benzoic acid, methyl ester,
4-[[1-[[3-(1,1-dimethylethyl)phenyl]sulfonyl]-3-fluoro-5-(trifluoromethyl)-1H-indol-2-yl]methyl]-benzoic acid,
4-[[[1-[3,3-dimethyl-2,3-dihydro-benzofuran-5-sulfonyl]-5-(chloro)-1H-indol-2-yl]methyl]benzoic acid, methyl ester,
4-[[[1-[3,3-dimethyl-2,3-dihydro-benzofuran-5-sulfonyl]-5-(chloro)-1H-indol-2-yl]methyl]benzoic acid,
4-[[1-[[3-(1,1-dimethylethyl)phenyl]sulfonyl]-3-methyl-5-(trifluoromethyl)-1H-indol-2-yl]methyl]-benzoic acid,
5-[[1-[[3-(1,1-dimethylethyl)-phenyl]sulfonyl]-5-trifluoromethyl-1H-indol-2-yl]-methyl]-thiophene-2-carboxylic acid, methyl ester,
3-[[1-[[3-(1,1-dimethylethyl)-phenyl]sulfonyl]-5-trifluoromethyl-1H-indol-2-yl]methyl]-4-fluoro-benzoic acid,
3-[[1-[[3-(1,1-dimethylethyl)-phenyl]sulfonyl]-5-trifluoromethyl-1H-indol-2-yl]methyl]-5-fluoro-benzoic acid,
3-[[1-[[3-(1,1-dimethylethyl)-phenyl]sulfonyl]-5-trifluoromethyl-1H-indol-2-yl]methyl]-5-fluoro-benzoic acid,
3-[[1-[[3-(1,1-dimethylethyl)-phenyl]sulfonyl]-5-trifluoromethyl-1H-indol-2-yl]methyl]-6-methoxy-benzoic acid,
3-[[1-[[3-(1,1-dimethylethyl)-phenyl]sulfonyl]-5-trifluoromethyl-1H-indol-2-yl]methyl]-4-chloro-6-fluoro-benzoic acid,
3-[[1-[[3-(1,1-dimethylethyl)-phenyl]sulfonyl]-5-trifluoromethyl-1H-indol-2-yl]methyl]-5-pyridine carboxylic acid,
4-[[1-[[3-(1,1-dimethylethyl)-phenyl]sulfonyl]-5-trifluoromethyl-1H-indol-2-yl]methyl]-2-chloro-benzoic acid,
3-[[1-[[3-(1,1-dimethylethyl)-phenyl]sulfonyl]-5-trifluoromethyl-1H-indol-2-yl]methyl]-6-fluoro-benzoic acid,
3-[[1-[[4-methyl-3,4-dihydro-2H-benzo[1,4]oxazin-6-yl]-sulfonyl]-5-trifluoromethyl-1H-indol-2-yl]methyl]-6-fluoro-benzoic acid,
4-[[1-[[3-(1,1-dimethylethyl)phenyl]sulfonyl]-5-(trifluoromethyl)-1H-indol-2-yl]fluoromethyl]benzoic acid,
4-[1-[3-(1,1-dimethylethyl)phenyl]sulfonyl]-5-(trifluoromethyl)-1H-indol-2H-tetrazol-5-yl-benzyl,
N-[4-[[1-[[3-(1,1-dimethylethyl)phenyl]sulfonyl]-5-(trifluoromethyl)-1H-indol-2-yl]methyl]benzyl]-methanesulfonamide; and the pharmaceutically acceptable salts of these compounds.

The compounds of formula I according to the invention in which R5 and R6 represent a hydrogen atom can be prepared according to a first method consisting of:

a) reacting the compound of formula II

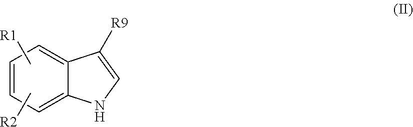

in which:
R1 and R2 each represent, independently of one another, a hydrogen atom, a halogen atom, a nitro group, an alkyl group having 1 to 4 carbon atoms, optionally fully or partially halogenated, an alkoxy group having 1 to 4 carbon atoms, a group —SCH₃, —OCF₃, a heterocyclic group having 4 to 6 atoms, —NH₂, —NHR, or —NR₂,
R represents a hydrogen atom or an alkyl group having 1 to 4 carbon atoms;
R9 represents a hydrogen atom, a halogen atom, an alkyl group having 1 to 4 carbon atoms; with a compound of formula (III)

R8SO₂Cl (III)

in which:
R8 represents an alkyl group having 1 to 6 carbon atoms, an aryl or heteroaryl group, substituted or unsubstituted, a cyclic or heterocyclic group, substituted or unsubstituted; in the presence of a solvent, for example tetrahydrofuran, and of a base, for example sodium hydride, at room temperature, for about 2 to 24 hours, to obtain the compound of formula IV:

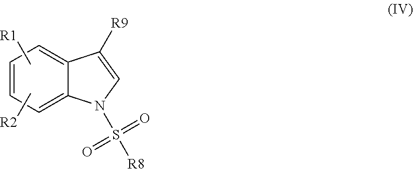

in which:
R1, R2, R8 and R9 retain the same meanings as in the starting compounds;

b) reacting the compound of formula IV with a borate of formula B(OAlk)₃ in which Alk represents an alkyl group having 1 to 4 carbon atoms, such as in particular B(OiPr)₃, in the presence of a base, such as in particular butyl-lithium (BuLi) or lithium diisopropyl amide (LDA), and of a solvent such as tetrahydrofuran or ether, at a temperature from about −100° C. to room temperature, preferably at −78° C., for a time from about 1 to 24 hours, preferably 18 hours, to obtain the compound of formula V:

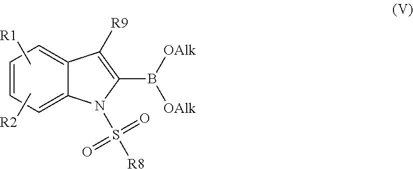

in which R1, R2, R8, R9 and Alk retain the same meanings as in the starting compounds;

c) reacting the compound V thus obtained with a compound of formula VI

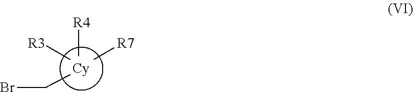

in which R3 and R4 each represent, independently of one another, a hydrogen atom, a halogen atom, an alkyl group having 1 to 4 carbon atoms, a hydroxyl group, an alkoxy group having 1 to 4 carbon atoms;
R7 represents a —COOR group in which R represents a hydrogen atom or an alkyl group having 1 to 4 carbon atoms, a bioisosteric group of carboxylic acid, or a —CN group, and Cy represents a phenyl group or a heteroaromatic group having 5 or 6 ring members;
in the presence of a base such as sodium carbonate, of a solvent such as in particular a dimethyl ether/water or ethanol/water mixture, and of a source of palladium such as in particular tetrakis(triphenylphosphine)palladium, to obtain the compound of formula Ia

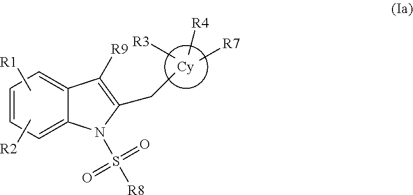

in which:
R1, R2, R3, R4, R7, R8, R9 and Cy retain the same meanings as in the starting compounds;

d) if necessary, hydrolysing the ester function of the compound of formula (Ia), for example by the action of a mineral base such as lithia according to procedures well known by a person skilled in the art, to obtain, after acid treatment, the compound of formula Ib in its free

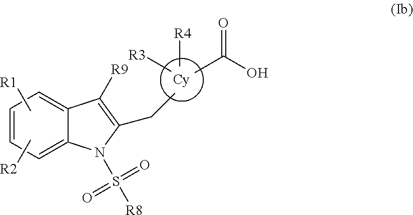

The compounds of formula I according to the invention in which R9 is a hydrogen atom can also be prepared according to a second method consisting of:

a) reacting the compound of formula VII:

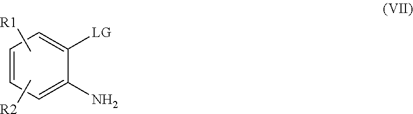

in which R1 and R2 each represent, independently of one another, a hydrogen atom, a halogen atom, a nitro group, an alkyl group having 1 to 4 carbon atoms, optionally fully or partially halogenated, an alkoxy group having 1 to 4 carbon atoms, a group —SCH₃, —OCF₃, a heterocyclic group having 4 to 6 atoms, —NH₂, —NHR or —NR₂;
LG represents an iodine atom, a bromine atom, a tosylate group or a trifluoromethane sulfonate group and R represents a hydrogen atom or a linear or branched alkyl group having 1 to 4 carbon atoms;
with the compound of formula III as defined previously,
in a solvent such as for example pyridine, at room temperature for a time of 3 to 48 hours, to obtain the compound of formula VIII:

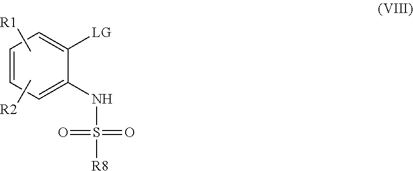

in which R1, R2, R8 and LG retain the same meanings as in the starting compounds;

b) reacting the compound of formula VIII with an acetylene derivative of formula IX:

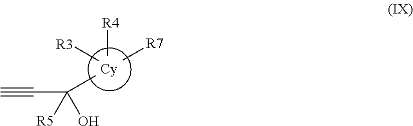

in which R3 and R4 each represent, independently of one another, a hydrogen atom, a halogen atom, an alkyl group having 1 to 4 carbon atoms, a hydroxyl group, an alkoxy group having 1 to 4 carbon atoms;
R5 represents a hydrogen atom, an alkyl group having 1 to 4 carbon atoms;
R7 represents a —COOR group, a bioisosteric group of carboxylic acid or a —CN group, in which R represents a hydrogen atom or an alkyl group having 1 to 4 carbon atoms; and
Cy represents a phenyl group or a heteroaromatic group having 5 or 6 ring members; in the presence of cuprous iodide, of a catalyst based on palladium such as for example bis(triphenylphosphine)palladium chloride, and of an organic base such as diethylamine or triethylamine, in a solvent for example dimethylformamide, under reflux, for 30 minutes to 8 hours, to obtain the compound of formula Ic:

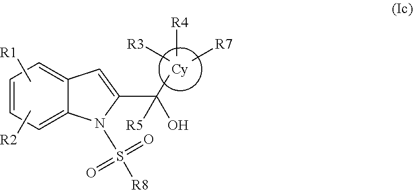

in which R1, R2, R3, R4, R5, R7, R8 and Cy retain the same meanings as in the starting compounds;

c) if necessary either reducing the compound of formula Ic thus obtained or eliminating the hydroxyl group of said compound by treatment with a mixture of triethylsilane, diethyl etherate of boron trifluoride and an optional catalytic amount of trifluoroacetic acid in a solvent such as dichloromethane, at room temperature, for a time from a few minutes to 24 hours, or according to other methods of reduction well known by a person skilled in the art such as treatment with zinc in an acid medium after chlorination; or oxidizing the compound of formula Ic by treatment with pyridinium dichromate in dichloromethane at room temperature for a time from 1 hour to 24 hours; or substituting the hydroxyl group with a fluorine atom by treatment with diethylaminosulfide trifluoride (DAST) in dichloromethane, to obtain the compound of formula If

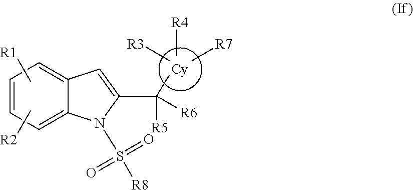

in which:
R1, R2, R3, R4, R7, R8 and Cy retain the same meaning as in the starting compound;
R5 and R6 each represent, independently of one another, a hydrogen atom, a halogen atom, an alkyl group having 1 to 4 carbon atoms, a hydroxyl group;
or R5 and R6 form, together with the carbon atom to which they are attached, a cycloalkyl group having 3 to 6 carbon atoms, an ethylene group (C═CH₂) or a carbonyl group (C═O):

d) if necessary, hydrolysing the ester function of the compound of formula If, for example by the action of a mineral base such as lithia according to procedures well known by a person skilled in the art, to obtain, after acid treatment, the compound of formula Id in its free acid form:

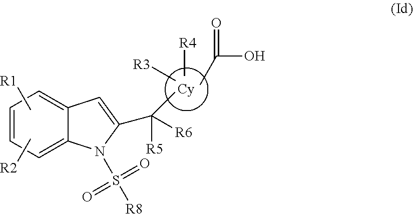

According to one embodiment of this second method, the aforementioned compound of formula VIII can be obtained from the aforementioned compound of formula VII by a method of sulfonylation comprising passage with a disulfonylated compound of formula X:

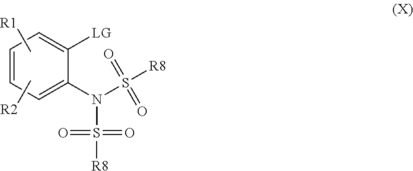

in which R1, R2, R8 and LG have the same meaning as stated previously.

According to this embodiment:
in a first stage, a mixture is formed in variable proportions of the monosulfonylated product of formula VIII and of the disulfonylated product of formula X by a treatment identical to that described in stage a) of the second method, but the reaction being carried out for a much longer time which can be up to 3 weeks; then
in a second stage, the raw reaction product thus obtained is treated directly with potassium in a solvent such as in particular dioxane for a time from about 2 to 24 hours.

The aforementioned compounds of formula IX can be obtained by reacting a compound of formula XXII:

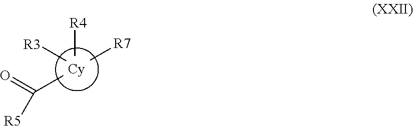

in which R3, R4, R5, R7 and Cy have the same meaning as in product IX,
with ethynylmagnesium bromide at a temperature of 0° C. for a time from 10 minutes to 18 hours.

The compounds of formula I according to the invention in which R9 represents a hydrogen atom or a halogen atom and R7 is a carboxyl —COOH group can also be prepared according to a third method consisting of:

a) reacting the compound of formula VII as defined previously with an acetylene derivative of formula XI:

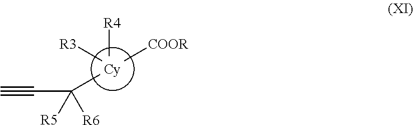

in which R3 and R4 each represent, independently of one another, a hydrogen atom, a halogen atom, an alkyl group having 1 to 4 carbon atoms, a hydroxyl group, an alkoxy group having 1 to 4 carbon atoms;
R5 and R6 each represent, independently of one another, a hydrogen atom, an alkyl group having 1 to 4 carbon atoms, a hydroxyl group;
or R5 and R6 form, together with the carbon atom to which they are attached, a cycloalkyl group having 3 to 6 carbon atoms, an ethylene group (C═CH₂) or a carbonyl group (C═O);
R represents an alkyl group having 1 to 4 carbon atoms, and
Cy represents a phenyl group or a heteroaromatic group having 5 or 6 ring members;
in the presence of cuprous iodide, of a catalyst based on palladium such as for example bis(triphenylphosphine)palladium chloride, and of an organic base such as diethylamine or triethylamine, in a solvent for example dimethylformamide, under reflux, for 30 minutes to 8 hours, to obtain the compound of formula XII:

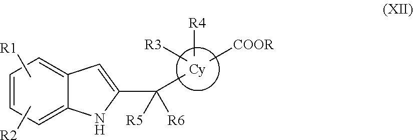

in which R1, R2, R3, R4, R5, R6, R and Cy retain the same meanings as in the starting compounds;

b) if necessary reacting the compound of formula XII with a halogenating agent for example 1-chloromethyl-4-fluoro-1,4-diazoniabicyclo[2.2.2]octane bis(tetrafluoroborate) at room temperature for about 30 minutes to 2 hours; to obtain the compound of formula XXII

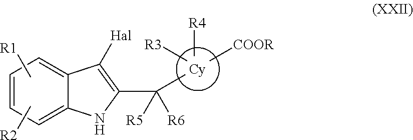

in which R1, R2, R3, R4, R5, R6, R and Cy retain the same meanings as in the starting compounds;
Hal represents a halogen atom;

c) reacting the compound of formula XII or the compound of formula XXII thus obtained with the compound of formula III as defined previously, in the presence of a solvent such as N-methylpyrrolidone (NMP) or dimethylformamide (DMF), and of a base, for example sodium hydride, at room temperature, for about 2 to 24 hours, preferably 18 hours, to obtain the compound of formula Ig;

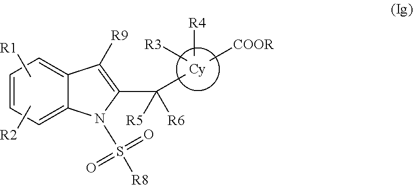

in which R1, R2, R3, R4, R5, R6, R8 and Cy retain the same meanings as in the starting compounds and
R9 represents a hydrogen atom or a halogen atom;

d) treating the reaction product thus obtained with lithium hydroxide in a solvent such as tetrahydrofuran, at room temperature, for about 2 to 24 hours, preferably 18 hours, to obtain the compound of formula Ik:

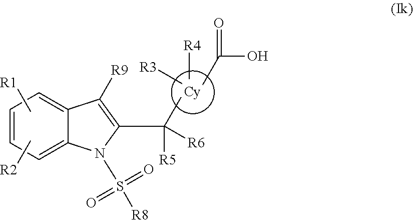

in which R1, R2, R3, R4, R5, R6, R8, R9 and Cy retain the same meanings as in the starting compounds.

The compounds of formula I according to the invention in which R9 represents a hydrogen atom and R7 is a carboxyl —COOH group can also be prepared according to a fourth method consisting of:

a) reacting the compound of formula VIII with the acetylene derivative of formula XI as defined previously, in the presence of cuprous iodide, of a catalyst based on palladium such as for example bis(triphenylphosphine)palladium chloride, and of an organic base such as diethylamine or triethylamine, in a solvent for example dimethylformamide, under reflux, for 30 minutes to 8 hours, to obtain the compound of formula Ie:

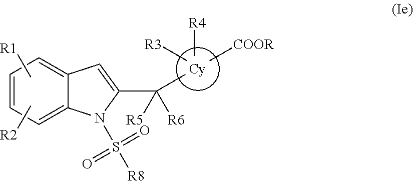

in which R1, R2, R3, R4, R5, R6, R8, R and Cy retain the same meanings as in the starting compounds;

b) reacting the compound of formula Ie thus obtained with lithium hydroxide in a solvent such as tetrahydrofuran, at room temperature, for about 2 to 24 hours, preferably 18 hours, to obtain the compound of formula Id as defined previously:

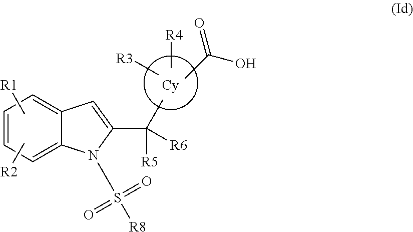

Certain compounds according to the invention can also be prepared according to a fifth method consisting of:

a) reacting the aforementioned compound of formula IV with an aldehyde derivative of formula XIII:

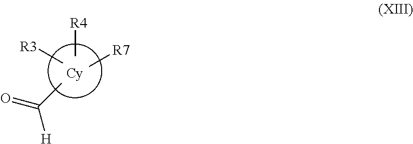

in which:
R3 and R4 each represent, independently of one another, a hydrogen atom, a halogen atom, an alkyl group having 1 to 4 carbon atoms, a hydroxyl group, an alkoxy group having 1 to 4 carbon atoms;
R7 represents a —COOR group in which R represents a hydrogen atom or an alkyl group having 1 to 4 carbon atoms, a bioisosteric group of carboxylic acid or a —CN group; and
Cy represents a phenyl group or a heteroaromatic group having 5 or 6 ring members;
in the presence of a base, such as in particular butyl-lithium (BuLi) or lithium diisopropyl amide (LDA), and of a solvent such as tetrahydrofuran or ether, at a temperature from about −78° C. to 0° C., preferably at −8° C., for a time from about 1 to 24 hours, preferably 2 hours, to obtain the compound of formula Ij:

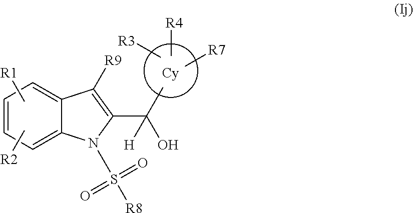

in which:
R1, R2, R3, R4, R7, R8, R9 and Cy retain the same meanings as in the starting compounds;

b) if necessary reducing or oxidizing the compound of formula Ij according to a treatment identical to that described in stage c) of the second method, to obtain the compound of formula I

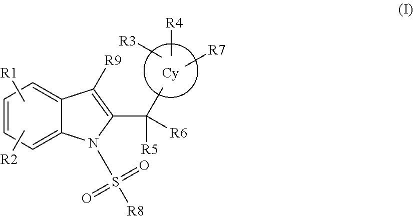

in which:
R1, R2, R3, R4, R7, R8, R9 and Cy retain the same meaning as in the starting compound; and
R5 and R6 each represent, independently of one another, a hydrogen atom, a halogen atom, a hydroxyl group;
or R5 and R6 form, together with the carbon atom to which they are attached, a carbonyl group (C═O);

c) if necessary, hydrolysing the ester function of the compound of formula (I), for example by the action of a mineral base such as lithia according to procedures well known by a person skilled in the art, to obtain, after acid treatment, the compound of formula Ik in its free acid form:

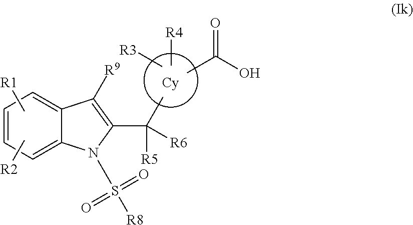

in which R1, R2, R3, R4, R5, R6, R8, R9 and Cy retain the same meaning as in the starting compound.

Certain compounds according to the invention can also be prepared according to a sixth method consisting of:

a) reacting the aforementioned compound of formula IV with an aldehyde derivative of formula XIV:

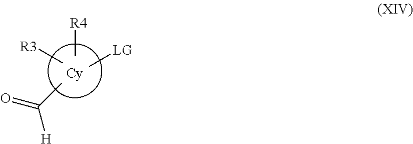

in which:
R3 and R4 each represent, independently of one another, a hydrogen atom, a halogen atom, an alkyl group having 1 to 4 carbon atoms, a hydroxyl group or an alkoxy group having 1 to 4 carbon atoms;
Cy represents a phenyl group or a heteroaromatic group having 5 or 6 ring members; and LG represents an iodine atom, a bromine atom or a tosylate or trifluoromethane-sulfonate group;
in the presence of a base, such as in particular butyl-lithium (BuLi) or lithium diisopropyl amide (LDA), and of a solvent such as tetrahydrofuran or ether, at a temperature from about −78° C. to 0° C., preferably at −8° C., for a time from about 1 to 24 hours, preferably 2 hours, to obtain the compound of formula XV:

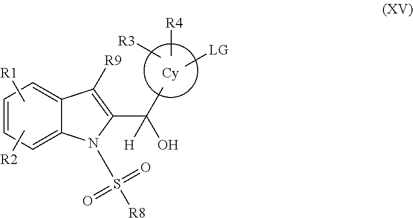

in which:
R1, R2, R3, R4, R8, R9, Cy and LG retain the same meanings as in the starting compounds;

b) if necessary reducing or oxidizing the compound of formula XV according to a treatment identical to that described in stage c) of the second method, to obtain the compound of formula XVI

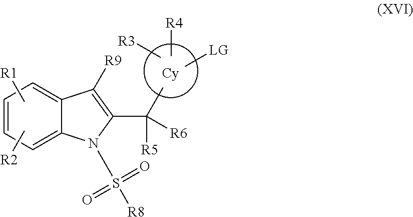

in which:
R1, R2, R3, R4, R8, R9, LG and Cy retain the same meaning as in the starting compound; and
R5 and R6 each represent, independently of one another, a hydrogen atom, a halogen atom, a hydroxyl group;
or R5 and R6 form, together with the carbon atom to which they are attached, a carbonyl group (C═O);

c) treating the compound of formula XVI with molybdenum hexacarbonyl in the presence of a catalyst based on palladium such as for example palladium acetate, of a ligand of the phosphine type such as for example tritert-butyl phosphine, of a mineral base such as sodium carbonate, in a solvent for example dimethoxyethane, under reflux, for 30 minutes to 48 hours, to obtain the compound of formula Ik

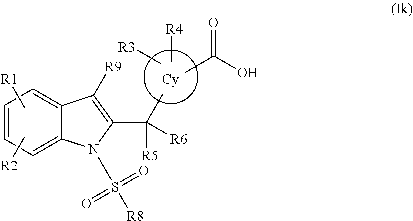

in which R1, R2, R3, R4, R5, R6, R8, R9 and Cy retain the same meaning as in the starting compound.

The compounds of formula (I) according to the invention in which R5, R6, and R9 represent a hydrogen atom can be prepared according to a seventh method consisting of:

a) reacting the compound of formula VIII prepared according to stage a) of the aforementioned second method with prop-2-yn-1-ol in the presence of cuprous iodide and of a catalyst based on palladium, for example bis(triphenyl phosphine) palladium(II) chloride, and of an organic base for example dimethylamine or triethylamine, in a suitable solvent for example N,N-dimethylformamide, at a temperature between room temperature and the reflux temperature of the solvent, for a time between 30 minutes and 6 hours, to obtain the compound

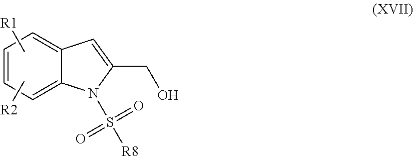

of formula XVII:
in which R1, R2 and R8 retain the same meanings as in the starting compounds;

b) reacting the aforementioned compound of formula XVII with a source of bromine, for example phosphorus tribromide, in a suitable solvent, for example dichloromethane, at room temperature, for a time from about 1 to 6 hours, to obtain the compound of formula XVII:

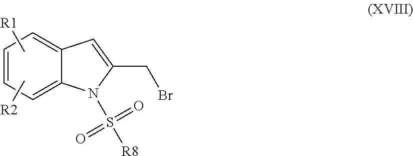

in which R1, R2 and R8 retain the same meanings as in the starting compounds;

c) reacting the aforementioned compound of formula XVIII with a compound of formula XIX:

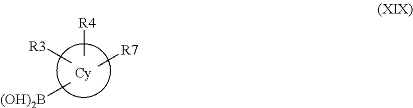

in which:
Cy represents a phenyl group or a heteroaromatic group having five or six ring members;
R3 and R4 each represent, independently of one another, a hydrogen atom, a halogen atom, an alkyl group having 1 to 4 carbon atoms, a hydroxyl group, an alkoxy group having 1 to 4 carbon atoms; and
R7 represents a —COOR group, a bioisosteric group of carboxylic acid or a —CN group; in a suitable solvent, for example a mixture of ethanol and dioxane, in the presence of a catalyst based on palladium, for example the Pd(dppf)Cl₂.CH₂Cl₂ complex; and of a suitable base, for example potassium carbonate, at a temperature between room temperature and the reflux temperature of the solvent, for a time from about 1 to 6 hours, to obtain the compound of formula Il:

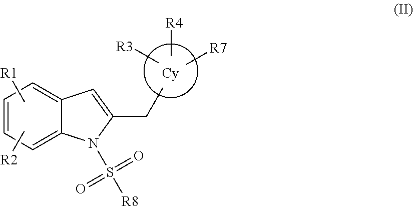

in which R1, R2, R3, R4, R7, R8 and Cy retain the same meanings as in the starting compounds.

The compounds of formulae I according to the invention, in which R3, R4, R5, R6 and R9 represent a hydrogen atom, Cy represents a thiazolyl group and R7 represents a COOH group can be prepared according to an eighth method consisting of:

a) reacting the aforementioned compound of formula XVIII with potassium cyanide in a suitable solvent, for example dichloromethane, in the presence of a phase transfer catalyst, for example tetrabutylammonium bromide, at room temperature for a time from 8 to 24 hours, so as to obtain the compound of formula XX:

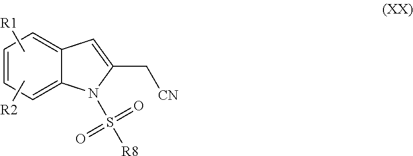

in which R1, R2 and R8 retain the same meanings as in the starting compounds;

b) reacting the compound of formula XX in solution in a suitable solvent, for example a mixture of tetrahydrofuran and water, with diethyl dithiophosphate at a temperature between about 80° C. and 120° C. for a time from about 1 to 6 hours, to obtain the compound of formula XXI:

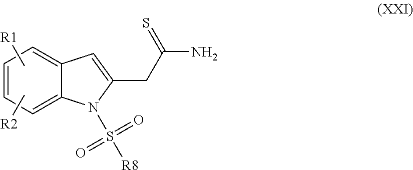

in which R1, R2 and R8 retain the same meanings as in the starting compounds;

c) reacting the compound of formula XXI with ethyl bromopyruvate, in a suitable solvent, for example ethanol, at room temperature for a time from about 12 to 36 hours, so as to obtain the compound of formula Ih:

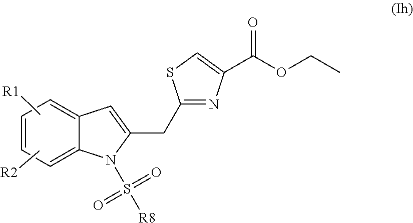

in which R1, R2 and R8 retain the same meanings as in the starting compounds;

d) if necessary, hydrolysing the ester function of the compound of formula Ih, for example by the action of a mineral base such as lithia according to procedures well known by a person skilled in the art, to obtain, after acid treatment, the compound of formula Ii in its free acid form:

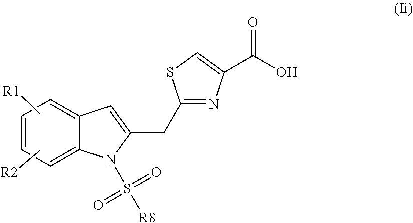

in which R1, R2 and R8 retain the same meanings as in the starting compounds.

The carboxylic acid function of the compounds of formula Ib, Id and Ik can be replaced advantageously with a bioisosteric group of carboxylic acid according to methods well known by a person skilled in the art such as the methods described below.

The compounds of formula I according to the invention, in which R7 represents a acylhydrazine, acylhydrazine carboxylate or oxadiazolone bioisosteric group can be prepared according to a method consisting of:

a) reacting the compound of formula Ib, Id, Ii or Ik on a carbazate in the presence of a coupling agent such as notably the reagent pair 1-(3-dimethylaminopropyl)-3-ethyl-carbodiimide (EDCI)/1-hydroxy-7-azabenzotriazole (HOAT), in an organic solvent such as in particular toluene at room temperature and for 2 to 24 hours to lead to the acylhydrazine carboxylate of formula Im

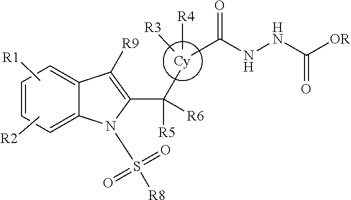

in which R1, R2, R3, R4, R5, R6, R8, R9 and Cy retain the same meanings as in the starting compounds; and
R represents a hydrogen atom or an alkyl group having 1 to 4 carbon atoms;

b) if necessary deprotecting the aforementioned compound of formula Im according to a procedure well known by a person skilled in the art for example by treating the compound of formula Im with an acid such as trifluoroacetic acid in a solvent such as in particular dichloromethane, to obtain a acylhydrazine;

c) if necessary cyclizing the acylhydrazine in the presence of a condensation agent such as carbonyldiimidazole (CDI) in an organic solvent such as dichloromethane, at room temperature and for 2 to 15 hours to obtain the oxadiazolone of formula In:

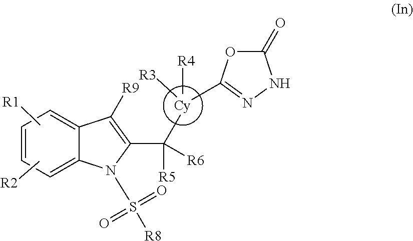

The compounds of formulae I according to the invention, in which R7 represents a sulfonylcarbamoyl bioisosteric group or a derivative group can be prepared according to a method consisting of coupling the compound of formula Ib, Id, Ii or Ik with a sulfonamide in the presence of a coupling agent such as in particular the reagent pair 1-(3-dimethylaminopropyl)-3-ethyl-carbodiimide hydrochloride/4-dimethylaminopyridine (EDCI/DMAP) in an organic solvent such as dichloromethane at room temperature for 12 to 24 hours.

The compounds of formulae I according to the invention, in which R7 represents an isoxazole bioisosteric group or a derivative group such as an isoxazolone group can be prepared according to a method consisting of:

a) activating the acid function of the compound of formula Ib, Id, Ii or Ik using carbonyldiimidazole (CDI) and reacting it with the magnesium salt of theethyl monomalonate;

b) cyclizing in the presence of hydroxylamine and in a basic medium at room temperature for 2 to 4 days to obtain the compound of formula Io:

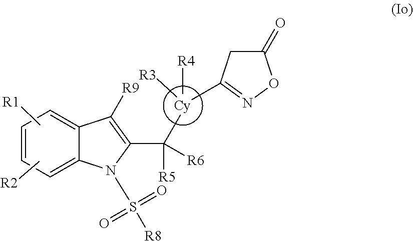

in which R1, R2, R3, R4, R5, R6, R8, R9 and Cy retain the same meanings as in the starting compounds;

The cyano function represented by R7 in the compounds of formula I or Il can advantageously be replaced with a bioisosteric group of carboxylic acid according to methods well known by a person skilled in the art such as the methods described below.

The compounds of formulae I according to the invention, in which R7 represents a tetrazole bioisosteric group can be prepared according to a method consisting of coupling the compound of formula I or Il, in which R7 represents a cyano group, with azidotrimethyltin in a solvent such as ortho-xylene, to form after 10 to 24 hours under reflux of the solvent, the tetrazole of formula Ip.

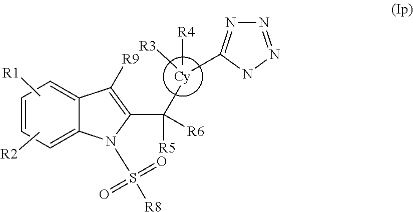

in which R1, R2, R3, R4, R5, R6, R8, R9 and Cy retain the same meanings as in the starting compounds;

The compounds of formula I according to the invention, in which R7 represents an oxadiazole bioisosteric group or a derivative group such as an oxadiazolone group can be prepared according to a method consisting of:

a) adding hydroxylamine sulfate on the cyano group of the compound of formula I or II in which R7 represents a cyano group, in the presence of triethylamine and of a solvent such as ethanol;

b) reacting the compound obtained with ethyl chloroformate for 18 to 24 hours under reflux of the solvent to obtain after acid treatment, the compound of formula Iq.

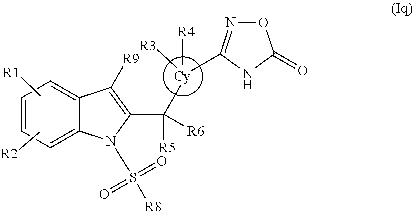

in which R1, R2, R3, R4, R5, R6, R8, R9 and Cy retain the same meanings as in the starting compounds;

We may also mention the compounds of formulae I according to the invention, in which R7 represents a thiazolidine bioisosteric group or a derivative group such as the thiazolidinedione group or the thioxothiazolidinone group which can be prepared according to a method consisting of carrying out a Knoevenagel condensation of a thiazolidine on the compound of formula XXIII in the presence of an inert solvent such as toluene, of a catalyst such as piperidine and in the presence of acetic acid.

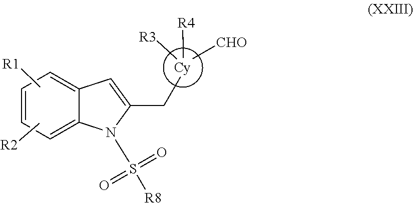

in which R1, R2, R3, R4, R8, and Cy retain the same meanings as in the starting compounds;

The compound of formula XXIII can be obtained according to a process identical to stage c) of the seventh method, by reacting the aforementioned compound of formula XVIII with the compound of formula XXIV

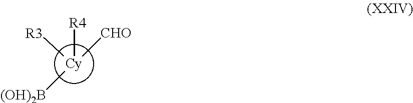

The compounds of the invention in the form of salts of the acids of formula Ib, Id, Ik, Ii with a mineral or organic base, can be obtained conventionally, using the methods that are well known by a person skilled in the art, for example by mixing stoichiometric amounts of the acid of formula Ib, Id, Ik, Ig, Ii and of the base in a solvent, such as for example water or a water-alcohol mixture, and then lyophilizing the solution obtained.

In some of the reaction stages described above, conventional methods of heating can be replaced advantageously with microwave heating using reactors adapted to this manner of reaction. In this case, a person skilled in the art will understand that the “heating” times will be greatly reduced, compared with the times required with conventional heating.

### EXAMPLES

The following examples of preparation of compounds according to formula I will make it easier to understand the invention.

In these examples, which do not limit the scope of the invention, “preparation” designates the examples describing the synthesis of intermediates and “examples” designates those describing the synthesis of compounds of formula (I) according to the invention.

The following abbreviations are used:
mM: millimole,
CH₃CN: acetonitrile,
DCM: dichloromethane,
DMAP: 4-dimethylaminopyridine,
DME: dimethoxyethane,
DMF: N,N-dimethylformamide,
DMSO: dimethylsulfoxide,
EDCI: 1-(3-dimethylaminopropyl)-3-ethyl-carbodiimide hydrochloride,
HOAt: 1-hydroxy-7-azabenzotriazole,
H₂O: water,
LiOH: lithium hydroxide,
MgSO₄: magnesium sulfate,
NH₄O: ammonium chloride,
NMP: N-methylpyrrolidone,
NaHCO₃: sodium hydrogen carbonate,
NaCl: sodium chloride,
Pd₂(dba)₃: dipalladium(0) tris(dibenzylideneacetone),
TFA: trifluoroacetic acid,
THF: tetrahydrofuran.

The melting points (m.p.) were measured using automatic equipment (Optimelt) and the spectral values of Nuclear Magnetic Resonance were characterized by the chemical shift (δ) calculated relative to TMS (tetramethylsilane), by the number of protons associated with the signal and by the form of the signal (s for singlet, d for doublet, t for triplet, q for quadruplet, m for multiplet, sept for septuplet, dd for doublet of doublets). The operating frequency (in MegaHertz) and the solvent used are stated for each compound.

Room temperature is 20° C.±5° C.

### Preparation I

### 1-[[4-(1-methylethyl)phenyl]sulfonyl]-5-(trifluoromethyl)-1H-indole

1.3 g (32.41 mM) of sodium hydride (at 60% in oil) was added portion by portion to a solution of 3.0 g (16.2 mM) of 5-trifluoromethyl-1H-indole in 30 mL of tetrahydrofuran. The reaction mixture was stirred for 30 minutes at room temperature, then 4.25 g (19.44 mM) of 4-(1-methylethyl)-benzenesulfonyl chloride in solution in 8 mL of tetrahydrofuran was added slowly. After stirring for 1.5 h, the reaction mixture was hydrolysed with water and extracted with ethyl acetate.

The organic phase was then washed with saturated NaCl aqueous solution, then dried over magnesium sulfate and evaporated under reduced pressure. The residue obtained was purified by silica gel chromatography, eluting with cyclohexane then progressively with a cyclohexane/ethyl acetate mixture (90/10; v/v). The fractions containing the expected product were combined and concentrated to dryness under reduced pressure to give 6.36 g of 1-[[4-(1-methylethyl)phenyl]sulfonyl]-5-(trifluoromethyl)-1H-indole as an orange solid (yield=69%).

¹H NMR (DMSOd₆, 300 MHz) δ=1.14 (d, 6H), 2.93 (sept, 1H), 6.98 (d, 1H), 7.49 (d, 2H), 6.68 (d, 1H), 7.96 (d, 2H), 8.01 (d, 1H), 8.06 (s, 1H), 8.17 (d, 1H).

### Preparation II

### 5-(chloro)-1-[[4-(1-methylethyl)phenyl]sulfonyl]-1H-indole

Working similarly to preparation I starting from 5-chloro-1H-indole, the expected product was obtained as a yellow liquid (quantitative yield).

¹H NMR (DMSOd₆, 250 MHz) δ=1.14 (d, 6H), 2.93 (sept, 1H), 6.82 (dd, 1H), 7.38 (dd, 1H), 7.47 (d, 2H), 7.70 (dd, 1H), 7.88 (d, 1H), 7.91 (d, 2H), 7.96 (m, 1H).

### Preparation III

### 1-[[3-(1,1-dimethylethyl)phenyl]sulfonyl]-5-(trifluoromethyl)-1H-indole

Working similarly to preparation I starting from 3-tert-butyl-benzenesulfonyl chloride, the expected product was obtained as a yellow solid (yield=98%)

### M.p.=85° C.

### Preparation IV

### 1-[[4-(1-methylethyl)phenyl]sulfonyl]-5-(trifluoromethyl)-1H-indole-2-boronic acid

14.03 mL (22.46 mM, c=1.6M in hexane) of butyl-lithium (BuLi) was added dropwise to a solution of 5.5 g (14.97 mM) of the compound obtained according to preparation I in 50 mL of tetrahydrofuran cooled to −78° C. The reaction mixture was heated to room temperature and stirred for an additional 20 minutes. After cooling to −78° C., 5.87 mL (25.45 mM) of triisopropyl borate was added. The reaction mixture was stirred at room temperature for 18 hours, hydrolysed with 150 mL of water and extracted with ethyl acetate. The organic phase was dried over magnesium sulfate and evaporated under reduced pressure to give 6.5 g of a green oil. The crude product was used without further purification in the next reaction.

### Preparation V

### 5-(chloro)-1-[[4-(1-methylethyl)phenyl]sulfonyl]-1H-indole-2-boronic acid

Working similarly to preparation IV starting from the compound obtained according to preparation II, the expected product was obtained and was used without further purification in the next reaction.

### Preparation VI

### 1-[[3-(1,1-dimethylethyl)phenyl]sulfonyl]-5-(trifluoromethyl)-1H-indole-2-boronic acid

Working similarly to preparation IV starting from the compound obtained according to preparation III, the expected product was obtained and was used without further purification in the next reaction.

### Example 1

### 2-fluoro-4-[[1-[[4-(1-methylethyl)phenyl]sulfonyl]-5-(trifluoromethyl)-1H-indol-2-yl]methyl]benzoic acid, methyl ester

A mixture of 900 mg (2.19 mM) of the compound obtained according to preparation IV, 540 mg (2.19 mM) of 4-(bromomethyl)-2-fluorobenzoic acid methyl ester, 126.46 mg (0.11 mM) of tetrakis(triphenylphosphine)palladium, 974.29 mg (9.19 mM) of sodium carbonate, 10 mL of water and 50 mL of ethylene glycol dimethyl ether was heated at reflux temperature for two hours. The reaction mixture was diluted with water and extracted twice with dichloromethane. The combined organic phases were dried over magnesium sulfate and evaporated under reduced pressure. The residue obtained was purified by silica gel chromatography, eluting with cyclohexane then progressively with a cyclohexane/ethyl acetate mixture (95/5; v/v). The fractions containing the expected product were combined and concentrated to dryness under reduced pressure to give 290 mg of 2-fluoro-4-[[1-[[4-(1-methylethyl)phenyl]sulfonyl]-5-(trifluoromethyl)-1H-indol-2-yl]methyl]benzoic acid methyl ester as a white solid (yield=25%). M.p.=132° C.

### Example 2

### 2-fluoro-4-[[1-[[4-(1-methylethyl)phenyl]sulfonyl]-5-(trifluoromethyl)-1H-indol-2-yl]methyl]benzoic acid

17 mg (0.40 mM) of lithium hydroxide was added to a solution of 180 mg (0.34 mM) of ester obtained according to example 1 in 16 mL of tetrahydrofuran and 4 mL of water. The reaction mixture was stirred for 7 hours at room temperature and then acidified with a solution of 1N hydrochloric acid. After extracting twice with dichloromethane, the combined organic phases were dried over magnesium sulfate, and evaporated under reduced pressure to give 175 mg of 2-fluoro-4-[[1-[[4-(1-methylethyl)phenyl]sulfonyl]-5-(trifluoromethyl)-1H-indol-2-yl]methyl]benzoic acid as a white solid (yield=99%). M.p.=197° C.

### Example 3

### 2-methoxy-4-[[1-[[4-(1-methylethyl)phenyl]sulfonyl]-5-(trifluoromethyl)-1H-indol-2-yl]methyl]benzoic acid, methyl ester

Working similarly to example 1, starting from 4-(bromomethyl)-2-methoxybenzoic acid methyl ester and the compound obtained according to preparation IV, the expected product was obtained as a yellow oil (yield=30%).

¹H NMR (DMSOd₆, 250 MHz) δ=1.13 (d, 6H), 2.91 (sept, 1H), 3.73 (s, 3H), 3.78 (s, 3H), 4.49 (s, 2H), 6.58 (s, 1H), 6.82 (dd, 1H), 7.02 (d, 1H), 7.39 (d, 2H), 7.58 (d, 1H), 7.65 (dd, 1H), 7.72 (d, 2H), 7.97 (s, 1H), 8.27 (d, 1H).

### Example 4

### 2-methoxy-4-[[1-[[4-(1-methylethyl)phenyl]sulfonyl]-5-(trifluoromethyl)-1H-indol-2-yl]methyl]benzoic acid

Working similarly to example 2, starting from the compound obtained according to example 3, the expected product was obtained as a beige oil (yield=98%).

¹H NMR (DMSOd₆, 250 MHz) δ=1.15 (d, 6H), 2.92 (sept, 1H), 3.73 (s, 3H), 4.48 (s, 2H), 6.57 (s, 1H), 6.82 (dd, 1H), 7.00 (d, 1H), 7.41 (d, 2H), 7.58 (d, 1H), 7.64 (dd, 1H), 7.73 (d, 2H), 7.96 (s, 1H), 8.27 (d, 1H), 12.51 (s broad, 1H).

### Example 5

### 4-[[5-chloro-1-[[4-(1-methylethyl)phenyl]sulfonyl]-1H-indol-2-yl]methyl]-2-fluorobenzoic acid, methyl ester

Working similarly to example 1, starting from 4-(bromomethyl)-2-fluorobenzoic acid methyl ester and the compound obtained according to preparation V, the expected product was obtained as yellow crystals (yield=12%). M.p.=127° C.

### Example 6

### 4-[[5-chloro-1-[[4-(1-methylethyl)phenyl]sulfonyl]-1H-indol-2-yl]methyl]-2-fluorobenzoic acid

Working similarly to example 2, starting from the compound obtained according to example 5, the expected product was obtained as white crystals (yield=34%). M.p.=196° C.

### Example 7

### 3-[[1-[[3-(1,1-dimethylethyl)phenyl]sulfonyl]-5-(trifluoromethyl)-1H-indol-2-yl]methyl]benzoic acid, methyl ester

Working similarly to example 1, starting from 3-(bromomethyl)-benzoic acid methyl ester and the compound obtained according to preparation VI, the expected product was obtained as an orange oil (yield=15%). ¹H NMR (DMSOd₆, 250 MHz) δ=1.17 (s, 9H), 3.83 (s, 3H), 4.51 (s, 2H), 6.55 (s, 1H), 7.50 (m, 3H), 7.68 (m, 4H), 7.81 (s, 1H), 7.86 (m, 1H), 7.95 (s, 1H), 8.28 (d, 1H).

### Example 8

### 3-[[1-[[3-(1,1-dimethylethyl)phenyl]sulfonyl]-5-(trifluoromethyl)-1H-indol-2-yl]methyl]benzoic acid

Working similarly to example 2, starting from the compound obtained according to example 7, the expected product was obtained as a beige crystalline powder (yield=95%). M.p.=146° C.

### Example 9

### 2-fluoro-4-[[1-[[3-(1,1-dimethylethyl)phenyl]sulfonyl]-5-(trifluoromethyl)-1H-indol-2-yl]methyl]benzoic acid, methyl ester

Working similarly to example 1, starting from 4-(bromomethyl)-2-fluorobenzoic acid methyl ester and the compound obtained according to preparation VI, the expected product was obtained as of an orange oil (yield=22%).

¹H NMR (DMSOd₆, 250 MHz) δ=1.16 (s, 9H), 3.85 (s, 3H), 4.53 (s, 2H), 6.67 (s, 1H), 7.18 (m, 2H), 7.47 (t, 1H), 7.68 (m, 4H), 7.83 (m, 1H), 7.97 (s, 1H), 8.27 (d, 1H).

### Example 10

### 2-fluoro-4-[[1-[[3-(1,1-dimethylethyl)phenyl]sulfonyl]-5-(trifluoromethyl)-1H-indol-2-yl]methyl]benzoic acid

Working similarly to example 2, starting from the compound obtained according to example 9, the expected product was obtained as beige crystals (yield=75%). M.p.=144° C.

### Example 11

### 4-[[1-[[3-(1,1-dimethylethyl)phenyl]sulfonyl]-5-(trifluoro methyl)-1H-indol-2-yl]methyl]-2-methoxybenzoic acid, methyl ester

Working similarly to example 1, starting from 4-(bromomethyl)-2-methoxybenzoic acid methyl ester and the compound obtained according to preparation VI, the expected product was obtained as a brown oil (yield=19%).

¹H NMR (DMSOd₆, 250 MHz) δ=1.17 (s, 9H), 3.76 (s, 3H), 3.78 (s, 3H), 4.48 (s, 2H), 6.54 (s, 1H), 6.83 (dd, 1H), 7.07 (d, 1H), 7.49 (t, 1H), 7.60 (d, 1H), 7.70 (m, 4H), 7.94 (s, 1H), 8.28 (d, 1H).

### Preparation VII

### 4-[[5-(trifluoromethyl)-1H-indol-2-yl]methyl]benzoic acid, methyl ester

In three separate reactors equipped for microwave heating, a mixture of 9.44 g (32.88 mM) of 2-iodo-4-trifluoromethyl-aniline (or 2-iodo-4-trifluoromethyl-benzeneamine), 6.3 g (36.17 mM) of methyl ester of 4-(2-propynyl)-benzoic acid, 1.15 g (1.64 mM) of bis-triphenylphosphine palladium (II) chloride, 0.31 g (1.64 mM) of copper iodide, 26.5 mL of triethylamine and 26.5 mL of dimethylformamide was heated 1×10 minutes at 120° C., then 2×3 minutes at 120° C. in microwave equipment. The combined reaction mixtures were evaporated under reduced pressure and the residue resulting was purified by silica gel chromatography, eluting with a cyclohexane/ethyl acetate mixture (95/5; v/v) then cyclohexane/ethyl acetate (90/10; v/v). The fractions containing the expected product were combined and concentrated to dryness under reduced pressure to give 6.3 g 4-[[5-(trifluoromethyl)-1H-indol-2-yl]methyl]benzoic acid methyl ester as a light yellow solid (yield=61%). M.p.=127° C.

### Example 12

### 4-[[1-[[4-(1,1-dimethylpropyl)phenyl]sulfonyl]-5-(trifluoromethyl)-1H-indol-2-yl]methyl]benzoic acid

A stock solution was prepared by mixing 2.9 g of the ester obtained according to preparation VII in solution in 14.5 mL of NMP and 696 mg of sodium hydride (60% suspension in oil) for 20 minutes. 500 μL of this solution was added to a solution of 148 mg of 4-(1,1-dimethylpropyl)-benzenesulfonyl chloride in 700 μL of NMP, and the reaction mixture was stirred for 18 hours at room temperature. The solvent was then evaporated under reduced pressure, and 500 μL of a saturated aqueous solution of ammonium chloride was added to the residue thus obtained and the reaction mixture was stirred for 15 minutes. 3 mL of ethyl acetate and 7 mL of a saturated aqueous solution of NaHCO₃ were added and the mixture thus obtained was stirred vigorously. The aqueous phase was extracted twice more with 1 mL of ethyl acetate. The organic phases were combined and evaporated under a nitrogen stream.

The residue thus formed was diluted with 5.4 mL of tetrahydrofuran and then treated with 1.2 mL of a stock solution of lithium hydroxide (prepared by dissolving 1.25 g of LiOH in 34.8 mL of water) at room temperature for 18 hours. The organic solvent was evaporated under a nitrogen stream, and the residue was diluted with 1 mL of an aqueous solution of 1N hydrochloric acid and extracted with a dichloromethane/methanol mixture (95/5; v/v). The organic phase was then evaporated under a nitrogen stream and the product was purified by semipreparative HPLC, thus obtaining 41 mg of 4-[[1-[[4-(1,1-dimethylpropyl)phenyl]-sulfonyl]-5-(trifluoromethyl)-1H-indol-2-yl]methyl]benzoic acid as a beige paste (yield=25%).

¹H NMR (DMSOd₆, 500 MHz) δ=0.52 (t, 3H), 1.18 (s, 6H), 1.55 (q, 2H), 4.51 (s, 2H), 6.60 (s, 1H), 7.29 (d, 2H), 7.45 (d, 2H), 7.65 (d, 1H), 7.69 (d, 2H), 7.85 (d, 2H), 7.98 (s, 1H), 8.27 (d, 1H), 12.92 (s broad, 1H).

Working similarly to example 12, starting from the corresponding sulfonyl chloride derivative, the compounds in the following examples 13 to 26 were obtained.

### Example 13

### 4-[[1-[(3-methoxyphenyl)sulfonyl]-5-(trifluoromethyl)-1H-indol-2-yl]methyl]benzoic acid

Appearance: beige paste Yield: 32%

¹H NMR (DMSOd₆, 500 MHz) δ=3.74 (s, 3H), 4.52 (s, 2H), 6.62 (s, 1H), 7.17 (m, 1H), 7.25 (m, 1H), 7.33 (d, 2H), 7.36 (m, 1H), 7.46 (t, 1H), 7.65 (d, 1H), 7.87 (d, 2H), 7.98 (s, 1H), 8.24 (d, 1H), 12.98 (s broad, 1H).

### Example 14

### 4-[[1-[(5-phenyl-2-thienyl)sulfonyl]-5-(trifluoromethyl)-1H-indol-2-yl]methyl]benzoic acid

Appearance: beige paste. Yield: 17%

¹H NMR (DMSOd₆, 500 MHz) δ=4.54 (s, 2H), 6.70 (s, 1H), 7.42 (m, 5H), 7.53 (d, 1H), 7.61 (m, 2H), 7.68 (d, 1H), 7.93 (m, 3H), 8.01 (s, 1H), 8.24 (d, 1H), 12.98 (s broad, 1H).

### Example 15

### 4-[[1-[(3-chloro-4-fluorophenyl)sulfonyl]-5-(trifluoromethyl)-1H-indol-2-yl]methyl]benzoic acid

Appearance: beige paste. Yield: 7%

¹H NMR (DMSOd₆, 500 MHz) δ=4.53 (s, 2H), 6.71 (s, 1H), 7.30 (d, 2H), 7.56 (t, 1H), 7.66 (d, 1H), 7.87 (m, 4H), 8.01 (s, 1H), 8.26 (d, 1H), 12.89 (s broad, 1H).

### Example 16

### 4-[[1-(3-thienylsulfonyl)-5-(trifluoromethyl)-1H-indol-2-yl]methyl]benzoic acid

Appearance: beige paste. Yield: 19%

¹H NMR (DMSOd₆, 500 MHz) δ=4.51 (s, 2H), 6.53 (s, 1H), 7.26 (d, 1H), 7.36 (d, 2H), 7.63 (d, 1H), 7.72 (m, 1H), 7.90 (d, 2H), 7.96 (s, 1H), 8.23 (d, 1H), 8.59 (s, 1H), 12.91 (s broad, 1H).

### Example 17

### 4-[[1-[(3,4-dihydro-2H-1,5-benzodioxepin-7-yl)sulfonyl]-5-(trifluoro-methyl)-1H-indol-2-yl]methyl]benzoic acid

Appearance: beige paste. Yield: 27%

¹H NMR (DMSOd₆, 250 MHz) δ=2.10 (q, 2H), 4.14 (t, 2H), 4.21 (t, 2H), 4.50 (s, 2H), 6.65 (s, 1H), 7.01 (d, 1H), 7.10 (d, 1H), 7.31 (d, 2H), 7.40 (dd, 1H), 7.65 (dd, 1H), 7.87 (d, 2H), 7.98 (s, 1H), 8.23 (d, 1H), 12.88 (s broad, 1H).

### Example 18

### 4-[[1-[[3-(1-methyl-1H-pyrazol-3-yl)phenyl]sulfonyl]-5-(trifluoromethyl)-1H-indol-2-yl]methyl]benzoic acid

Appearance: beige paste. Yield: 27%

¹H NMR (DMSOd₆, 500 MHz) δ=3.90 (s, 3H), 4.55 (s, 2H), 6.58 (s, 1H), 6.77 (d, 1H), 7.37 (d, 2H), 7.56 (t, 1H), 7.67 (m, 2H), 7.77 (d, 1H), 7.88 (d, 2H), 7.96 (s, 1H), 8.05 (m, 1H), 8.17 (m, 1H), 8.26 (d, 1H), 12.87 (s broad, 1H).

### Example 19

### 4-[[1-[(5,6,7,8-tetrahydro-5,5,8,8-tetramethyl-2-naphthalenyl)sulfonyl]-5-(trifluoromethyl)-1H-indol-2-yl]methyl]benzoic acid

Appearance: beige paste. Yield: 16%

¹H NMR (DMSOd₆, 500 MHz) δ=1.10 (s, 6H), 1.17 (s, 6H), 1.58 (s, 4H), 4.50 (s, 2H), 6.57 (s, 1H), 7.29 (d, 2H), 7.47 (m, 2H), 7.64 (s, 1H), 7.66 (d, 1H), 7.86 (d, 2H), 7.97 (s, 1H), 8.31 (d, 1H), 12.91 (s broad, 1H).

### Example 20

### 4-[[1-[[3-(1-methyl-1H-pyrazol-5-yl)phenyl]sulfonyl]-5-(trifluoromethyl)-1H-indol-2-yl]methyl]benzoic acid

Appearance: beige paste. Yield: 9%

¹H NMR (DMSOd₆, 500 MHz) δ=3.68 (s, 3H), 4.56 (s, 2H), 6.42 (d, 1H), 6.65 (s, 1H), 7.36 (d, 2H), 7.48 (d, 1H), 7.66 (m, 2H), 7.86 (m, 5H), 7.98 (s, 1H), 8.30 (d, 1H), 12.88 (s broad, 1H).

### Example 21

### 4-[[1-[[4-(1,1-dimethylethyl)phenyl]sulfonyl]-5-(trifluoromethyl)-1H-indol-2-yl]methyl]benzoic acid

Appearance: beige paste. Yield: 27%

¹H NMR (DMSOd₆, 500 MHz) δ=1.22 (s, 9H), 4.52 (s, 2H), 6.60 (s, 1H), 7.29 (d, 2H), 7.51 (d, 2H), 7.65 (d, 1H), 7.70 (d, 2H), 7.85 (d, 2H), 7.98 (s, 1H), 8.27 (d, 1H), 12.88 (s broad, 1H).

### Example 22

### 4-[[1-[(2,3-dihydro-1,4-benzodioxin-6-yl)sulfonyl]-5-(trifluoromethyl)-1H-indol-2-yl]methyl]benzoic acid

Appearance: beige paste. Yield: 24%

¹H NMR (DMSOd₆, 500 MHz) δ=4.23 (m, 2H), 4.27 (m, 2H), 4.50 (s, 2H), 6.61 (s, 1H), 6.96 (d, 1H), 7.12 (d, 1H), 7.32 (m, 3H), 7.64 (d, 1H), 7.88 (d, 2H), 7.97 (s, 1H), 8.24 (d, 1H), 12.90 (s broad, 1H).

### Example 23

### 4-[[1-[[3-(1,1-dimethylethyl)-4-(methoxy)phenyl]sulfonyl]-5-(trifluoro-methyl)-1H-indol-2-yl]methyl]benzoic acid

Appearance: beige paste. Yield: 32%

¹H NMR (DMSOd₆, 500 MHz) δ=1.19 (s, 9H), 3.84 (s, 3H), 4.49 (s, 2H), 6.56 (s, 1H), 7.06 (d, 1H), 7.29 (d, 2H), 7.48 (d, 1H), 7.66 (d, 1H), 7.70 (d, 1H), 7.86 (d, 2H), 7.96 (s, 1H), 8.27 (d, 1H), 12.90 (s broad, 1H).

### Example 24

### 4-[[1-(ethylsulfonyl)-5-(trifluoromethyl)-1H-indol-2-yl]methyl]benzoic acid

Appearance: beige paste. Yield: 9%

¹H NMR (DMSOd₆, 500 MHz) δ=1.06 (t, 3H), 3.52 (q, 2H), 4.42 (s, 2H), 6.50 (s, 1H), 7.39 (d, 2H), 7.62 (d, 1H), 7.91 (d, 2H), 8.01 (s, 1H), 8.07 (d, 1H), 12.94 (s broad, 1H).

### Example 25

### 4-[[1-(2-naphthalenylsulfonyl)-5-(trifluoromethyl)-1H-indol-2-yl]methyl]benzoic acid

Appearance: beige paste. Yield: 30%

¹H NMR (DMSOd₆, 250 MHz) δ=4.60 (s, 2H), 6.60 (s, 1H), 7.35 (d, 2H), 7.70 (m, 4H), 7.84 (d, 2H), 7.98 (m, 3H), 8.15 (d, 1H), 8.33 (d, 1H), 8.65 (s, 1H), 12.84 (s broad, 1H).

### Example 26

### 4-[[1-[[2-methyl-5-(1-methylethyl)phenyl]sulfonyl]-5-(trifluoromethyl)-1H-indol-2-yl]methyl]benzoic acid

Appearance: beige paste. Yield: 15%

¹H NMR (DMSOd₆, 250 MHz) δ=1.02 (d, 6H), 2.35 (s, 3H), 2.82 (sept, 1H), 4.36 (s, 2H), 6.67 (s, 1H), 7.09 (d, 1H), 7.22 (d, 2H), 7.31 (d, 1H), 7.44 (dd, 1H), 7.60 (dd, 1H), 7.82 (d, 2H), 8.02 (d, 1H), 8.06 (s, 1H), 12.86 (s broad, 1H).

### Preparation VIII

### 4-[(5-chloro-1H-indol-2-yl)methyl]benzoic acid, methyl ester

Working according to the procedure of preparation VII, starting from 4-chloro-2-iodo-aniline, the expected product was obtained as a beige solid (yield=50%). M.p.=118° C.

Working according to the procedure of example 12, starting from preparation VIII and the corresponding sulfonylated derivative, the following examples 27 to 29 were prepared.

### Example 27

### 4-[[5-chloro-1-[(4-chloro-3-methyl-phenyl)sulfonyl]-1H-indol-2-yl]-methyl]benzoic acid

Appearance: beige paste. Yield: 8%

¹H NMR (DMSOd₆, 250 MHz) δ=2.27 (s, 3H), 4.49 (s, 2H), 6.50 (s, 1H), 7.32 (m, 3H), 7.57 (m, 2H), 7.62 (d, 1H), 7.70 (m, 1H), 7.86 (d, 2H), 8.03 (d, 1H), 12.98 (s broad, 1H).

### Example 28

### 4-[[5-chloro-1-[[3-(trifluoromethyl)phenyl]sulfonyl]-1H-indol-2-yl]-methyl]benzoic acid

Appearance: beige paste. Yield: 14%

¹H NMR (DMSOd₆, 250 MHz) δ=4.50 (s, 2H), 6.55 (s, 1H), 7.32 (d, 2H), 7.37 (dd, 1H), 7.65 (d, 1H), 7.77 (t, 1H), 7.86 (d, 2H), 7.90 (s, 1H), 8.05 (m, 3H), 12.84 (s broad, 1H).

### Example 29

### 4-[[5-chloro-1-(3-thienylsulfonyl)-1H-indol-2-yl]methyl]benzoic acid

Appearance: beige paste. Yield: 17%

¹H NMR (DMSOd₆, 250 MHz) δ=4.46 (s, 2H), 6.37 (s, 1H), 7.21 (dd, 1H), 7.32 (dd, 1H), 7.36 (d, 2H), 7.60 (d, 1H), 7.70 (dd, 1H), 7.90 (d, 2H), 8.02 (d, 1H), 8.53 (dd, 1H), 12.89 (s broad, 1H).

### Preparation IX

### N-[2-iodo-4-(trifluoromethyl)-phenyl]-3-(1-methylethyl)benzenesulfonamide

67.78 g (309.92 mM) of 3-(1-methylethyl)-benzenesulfonyl chloride was added dropwise over a period of 10 minutes to a solution of 72 g (250.86 mM) of 2-iodo-4-trifluoromethyl-aniline in 216 mL of pyridine and the reaction mixture was stirred for 21 hours at room temperature. Next, 42.22 g (752.57 mM) of potassium hydroxide and then 250 mL of water and 125 mL of dioxane were added. After stirring for 5 hours at reflux temperature, then 64 hours at room temperature and a further 8 hours at reflux temperature, the reaction mixture was poured into 2 L a ice-water mixture and 325 mL of 10N hydrochloric acid, and extracted three times with 500 mL of ethyl acetate. The combined organic phases were dried over magnesium sulfate and evaporated under reduced pressure. The residue obtained was purified by silica gel chromatography, eluting with a cyclohexane/ethyl acetate mixture (90/10; v/v) then (80/20; v/v). The fractions containing the expected product were combined and concentrated to dryness under reduced pressure to give 128 g of N-[2-iodo-4-(trifluoromethyl)phenyl]-3-(1-methylethyl)-benzenesulfonamide as a beige solid (quantitative yield).

¹H NMR (DMSOd₆, 300 MHz) δ=1.15 (d, 6H), 2.94 (sept, 1H), 7.30 (d, 1H), 7.54 (m, 4H), 7.73 (dd, 1H), 8.11 (d, 1H), 9.99 (s broad, 1H).

### Example 30

### 4-[(RS)-hydroxy[1-[[3-(1-methylethyl)phenyl]sulfonyl]-5-(trifluoro-methyl)-1H-indol-2-yl]methyl]benzoic acid, methyl ester

A mixture of 117.72 g (250.86 mM) of N-[2-iodo-4-(trifluoromethyl)-phenyl]-3-(1-methylethyl)-benzenesulfonamide (preparation IX), 52.48 g (275.95 mM) of 4-(1-hydroxy-2-propynyl)benzoic acid methyl ester, 5.54 g (7.89 mM) of bis-triphenylphosphine palladium (II) chloride, 2.7 g (14.18 mM) of copper (cuprous) iodide, 150 mL of diethylamine and 500 mL of dimethylformamide was heated for 30 minutes at reflux temperature. The solvent was evaporated under reduced pressure and the residue was purified by silica gel chromatography, eluting with a cyclohexane/ethyl acetate mixture (95/5; v/v) then progressively with a cyclohexane/ethyl acetate mixture (70/30; v/v). The fractions containing the expected product were combined and concentrated to dryness under reduced pressure to give 102 g of 4-[(RS)-hydroxy[1-[[3-(1-methyl ethyl)phenyl]-sulfonyl]-5-(trifluoromethyl)-1H-indol-2-yl]methyl]-benzoic acid methyl ester as an orange oil (yield=82%).

¹H NMR (DMSOd₆, 300 MHz) δ=1.08 (d, 3H), 1.10 (d, 3H), 2.85 (sept, 1H), 3.86 (s, 3H), 6.50 (m, 2H), 6.80 (s, 1H), 7.53 (m, 7H), 7.95 (d, 2H), 8.01 (m, 1H), 8.23 (s, 1H).

### Example 31

### 4-[[1-[[3-(1-methylethyl)phenyl]sulfonyl]-5-(trifluoromethyl)-1H-indol-2-yl]methyl]benzoic acid, methyl ester

154.3 mL (966 mM) of triethylsilane, 10 mL of trifluoroacetic acid and 122.42 mL (966 mM) of boron trifluoride diethyl etherate were added successively, dropwise, to a solution of 102.7 g (193.21 mM) of ester obtained according to example 30 in 1 L of dichloromethane. The reaction mixture was stirred for 1 hour at room temperature, then poured slowly into 1 L of ice water. After decanting, the organic phase was washed successively with 0.5 L of water, 0.5 L of a saturated aqueous solution of potassium carbonate and 0.5 L of water, then dried over magnesium sulfate and evaporated under reduced pressure. The residue obtained was purified by silica gel chromatography, eluting with a cyclohexane/ethyl acetate mixture (95/5; v/v) then progressively up to cyclohexane/ethyl acetate mixture (80/20; v/v). The fractions containing the expected product were combined and concentrated to dryness under reduced pressure to give 78 g of 4-[[1-[[3-(1-methylethyl)phenyl]sulfonyl]-5-(trifluoromethyl)-1H-indol-2-yl]methyl]benzoic acid methyl ester as a pale yellow oil (yield=78%).

¹H NMR (DMSOd₆, 300 MHz) δ=1.08 (d, 6H), 2.86 (sept, 1H), 3.85 (s, 3H), 4.54 (s, 2H), 6.62 (s, 1H), 7.38 (d, 2H), 7.45 (t, 1H), 7.60 (m, 4H), 7.91 (d, 2H), 7.95 (m, 1H), 8.25 (d, 1H).

### Example 32

### 4-[[1-[[3-(1-methylethyl)phenyl]sulfonyl]-5-(trifluoromethyl)-1H-indol-2-yl]methyl]benzoic acid

Working similarly to example 2, starting from the compound of example 31, the expected product was obtained as a white solid (yield=88%). M.p.=175° C.

### Preparation X

### N-(2-iodo-4-(trifluoromethyl)-phenyl)-4-(1-methylethyl)-benzenesulfonamide

370 μL (2.09 mM) of 4-(1-methylethyl)benzenesulfonyl chloride was added to a solution of 0.5 g (1.74 mM) of 2-iodo-4-(trifluoromethyl)aniline in 5 mL of pyridine. The reaction mixture was stirred for 18 hours at room temperature, then poured into 5 mL of an aqueous solution of 1N hydrochloric acid. The mixture was extracted with 3×10 mL of ethyl acetate. The combined organic phases were dried over magnesium sulfate and concentrated under reduced pressure. The residue obtained was purified by silica gel chromatography, eluting with a cyclohexane/ethyl acetate mixture (90/10; v/v). The fractions containing the expected product were combined and concentrated to dryness under reduced pressure to give 430 mg of N-(2-iodo-4-trifluoromethyl-phenyl)-4-(1-methylethyl)-benzenesulfonamide as a yellow solid (yield=55%). M.p.=101° C.

Working similarly to preparation X, starting from the corresponding sulfonyl chloride derivative, the compounds of preparations XI and XII were obtained.

### Preparation XI

### N-(2-iodo-4-(trifluoromethyl)-phenyl)-3-(1,1-dimethylethyl)-benzene-sulfonamide

Appearance: white solid. Yield: 42%

¹H NMR (DMSOd₆, 300 MHz) δ=1.22 (s, 9H), 7.32 (d, 1H), 7.56 (m, 3H), 7.72 (m, 2H), 8.10 (d, 1H), 9.99 (s broad, 1H).

### Preparation XII

### N-[2-iodo-4-(trifluoromethyl)phenyl]-3,4-dihydro-4-methyl-2H-1,4-benzoxazine-6-sulfonamide

Appearance: orange solid. Yield: 81%. M.p.=127° C.

### Preparation XIII

### N-(2-iodo-4-(trifluoromethoxy)-phenyl)-4-(1-methylethyl)-benzenesulfonamide

Working similarly to preparation X starting from 2-iodo-4-(trifluoromethoxy)-aniline and 4-(1-methylethyl-)-benzenesulfonamide chloride, the expected compound was obtained as a brown solid (yield=91%). M.p.=72° C.

### Preparation XIV

### N-(4-chloro-2-iodo-phenyl)-4-(1-methylethyl)-benzenesulfonamide

Working similarly to preparation X starting from 2-iodo-4-chloro-aniline and 4-(1-methylethyl-)-benzenesulfonamide chloride, the expected compound was obtained as a white solid (yield=75%). M.p.=149° C.

### Example 33

### 4-[(RS)-hydroxy[1-[[4-(1-methylethyl)phenyl]sulfonyl]-5-(trifluoro-methoxy)-1H-indol-2-yl]methyl]benzoic acid, methyl ester

Working similarly to example 30 starting from the compound obtained in preparation XIII, the expected compound was obtained in the form of a yellow solid (yield=69%).

¹H NMR (DMSOd₆, 250 MHz) δ=1.13 (d, 6H), 2.91 (sept, 1H), 3.85 (s, 3H), 6.45 (m, 2H), 6.72 (s, 1H), 7.31 (m, 1H), 7.38 (d, 2H), 7.48 (d, 2H), 7.63 (m, 1H), 7.75 (d, 2H), 7.92 (d, 2H), 8.12 (d, 1H).

### Example 34

### 1-[[4-(1-methylethyl)phenyl]sulfonyl]-5-(trifluoromethoxy)-1H-indol-2-yl]methyl]benzoic acid, methyl ester

Working similarly to example 31, starting from the ester of example 33, the expected compound was obtained as a white solid (yield=81%). M.p.=103° C.

### Example 35

### 1-[[4-(1-methylethyl)phenyl]sulfonyl]-5-(trifluoromethoxy)-1H-indol-2-yl]methyl]benzoic acid

Working similarly to example 2, starting from the ester of example 34, the expected compound was obtained as a white solid (yield=76%). M.p.=66° C.

### Example 36

### 4-[hydroxy[1-[[4-(1-methylethyl)phenyl]sulfonyl]-5-(trifluoromethyl)-1H-indol-2-yl]methyl]benzoic acid, methyl ester

Working similarly to example 30, starting from the compound obtained in preparation X, the expected compound was obtained as a yellow solid (yield=83%). M.p.=68° C.

### Example 37

### 4-[[1-[(3,4-dihydro-4-methyl-2H-1,4-benzoxazin-6-yl)sulfonyl]-5-(trifluoromethyl)-1H-indol-2-yl]hydroxymethyl]benzoic acid, methyl ester

Working similarly to example 30, starting from the compound from preparation XII, the expected compound was obtained in the form of a yellow solid (yield=53%). M.p.=80° C.

### Example 38

### 4-[[5-chloro-1-[[4-(1-methylethyl)phenyl]sulfonyl]-1H-indol-2-yl]hydroxy methyl]benzoic acid, methyl ester

Working similarly to example 30, starting from 3-(1-hydroxy-prop-2-ynyl)-benzoic acid methyl ester and the compound obtained in preparation XIV, the expected compound was obtained as a yellow solid (yield=76%). M.p.=71° C.

### Preparation XV

### 4-[(1RS)-1-hydroxy-1-methyl-2-propynyl]benzoic acid, methyl ester

Under argon, 44.9 mL (22.45 mM) of ethynylmagnesium bromide was added to a solution of 2 g (11.22 mol) of 4-acetyl-benzoic acid methyl ester in solution in 40 mL of tetrahydrofuran and the mixture was stirred overnight at room temperature. The reaction mixture was diluted with a saturated aqueous solution of NH₄Cl and extracted 3 times with ethyl acetate. The combined organic phases were dried over magnesium sulfate and evaporated under reduced pressure. The residue was purified by silica gel chromatography, eluting with a cyclohexane/ethyl acetate mixture (80/20; v/v) and the fractions containing the expected product were combined and concentrated to dryness under reduced pressure to give 2.3 g of 4-(1-hydroxy-1-methyl-2-propynyl)benzoic acid methyl ester as a white solid (yield=33%).

¹H NMR (DMSOd₆, 300 MHz) δ=1.62 (s, 3H), 3.57 (s, 1H), 3.84 (s, 3H), 6.28 (s, 1H), 7.69 (d, 2H), 7.95 (d, 2H).

### Example 39

### 4-[1-[1-[[3-(1,1-dimethylethyl)phenyl]sulfonyl]-5-(trifluoromethyl)-1H-indol-2-yl]-1-hydroxyethyl]benzoic acid, methyl ester

Working similarly to example 30, starting from the ester obtained in preparation XV and the compound obtained in preparation XI, the expected compound was obtained as a beige solid (yield=70%). M.p.=70° C.

### Example 40

### 4-[[5-chloro-1-[[4-(1-methylethyl)phenyl]sulfonyl]-1H-indol-2-yl]methyl]benzoic acid, methyl ester

Working similarly to example 31, starting from the ester of example 38, the expected compound was obtained as a white solid (yield=74%). M.p.=99° C.

### Example 41

### 4-[[5-chloro-1-[[4-(1-methylethyl)phenyl]sulfonyl]-1H-indol-2-yl]methyl]benzoic acid

Working similarly to example 2, starting from the ester of example 40, the expected compound was obtained as a white solid (yield=79%). M.p.=192° C.

### Example 42

### 4-[[1-[[4-(1-methylethyl)phenyl]sulfonyl]-5-(trifluoromethyl)-1H-indol-2-yl]methyl]benzoic acid, methyl ester

Working similarly to example 31, starting from the ester of example 36, the expected compound was obtained as a pink solid (yield=72%). M.p.=123° C.

### Example 43

### 4-[[1-[[4-(1-methylethyl)phenyl]sulfonyl]-5-(trifluoromethyl)-1H-indol-2-yl]methyl]benzoic acid

Working similarly to example 2, starting from the ester of example 42, the expected compound was obtained as a white solid (yield=42%). M.p.=227° C.

### Example 44

### 4-[[1-[(3,4-dihydro-4-methyl-2H-1,4-benzoxazin-6-yl)sulfonyl]-5-(trifluoromethyl)-1H-indol-2-yl]methyl]benzoic acid, methyl ester

Working similarly to example 31, starting from the ester of example 37, the expected compound was obtained as a white solid (yield=74%). M.p.=63° C.

### Example 45

### 4-[[1-[(3,4-dihydro-4-methyl-2H-1,4-benzoxazin-6-yl)sulfonyl]-5-(trifluoromethyl)-1H-indol-2-yl]methyl]benzoic acid

Working similarly to example 2, starting from the ester of example 44, the expected compound was obtained as a white solid (yield=31%). M.p.=206° C.

### Example 46

### 4-[(RS)-1-[1-[[3-(1,1-dimethylethyl)phenyl]sulfonyl]-5-(trifluoromethyl)-1H-indol-2-yl]ethyl]benzoic acid, methyl ester

Working similarly to example 31, starting from the ester of example 39, the expected compound was obtained as a beige paste (yield=62%).

¹H NMR (DMSOd₆, 250 MHz) δ=1.13 (s, 9H), 1.62 (d, 3H), 3.83 (s, 3H), 5.03 (q, 1H), 7.01 (s, 1H), 7.28 (d, 2H), 7.43 (m, 2H), 7.54 (m, 1H), 7.67 (m, 2H), 7.83 (d, 2H), 7.99 (s, 1H), 8.25 (d, 1H).

### Example 47

### 4-[(RS)-1-[1-[[3-(1,1-dimethylethyl)phenyl]sulfonyl]-5-(trifluoromethyl)-1H-indol-2-yl]ethyl]benzoic acid

Working similarly to example 2, starting from the ester of example 46, the expected compound was obtained as white crystals (yield=65%). M.p.=212° C.

### Example 48

### 4-[[1-[[3-(1,1-dimethylethyl)phenyl]sulfonyl]-5-(trifluoromethyl)-1H-indol-2-yl]methyl]benzoic acid, methyl ester

A mixture of 250 mg (0.52 mM) of N-[2-iodo-4-(trifluoromethyl)-phenyl]-3-(1,1-dimethylethyl)-benzenesulfonamide obtained in preparation XI, 90 mg (0.52 mM) of 4-(2-propynyl)-benzoic acid methyl ester, 9.08 mg (0.01 mM) of bis-triphenylphosphine palladium (II) chloride, 4.93 mg (0.03 mM) of copper iodide, 2 mL of triethylamine and 2 mL of dimethylformamide was heated 2×20 minutes at 120° C. in microwave equipment. The reaction mixture was diluted in water and extracted with ethyl acetate. The organic phase was dried over magnesium sulfate and evaporated under reduced pressure. The residue obtained was purified by silica gel chromatography, eluting with cyclohexane/ethyl acetate then progressively with a cyclohexane/ethyl acetate mixture (80/20; v/v). The fractions containing the expected product were combined and concentrated to dryness under reduced pressure to give 83 mg of 4-[[1-[[3-(1,1-dimethylethyl)phenyl]sulfonyl]-5-(trifluoromethyl)-1H-indol-2-yl]methyl]benzoic acid methyl ester as a white solid (yield=38%).

¹H NMR (DMSOd₆, 300 MHz) δ=1.17 (s, 9H), 3.85 (s, 3H), 4.52 (s, 2H), 6.59 (s, 1H), 7.36 (d, 2H), 7.47 (t, 1H), 7.67 (m, 4H), 7.90 (d, 2H), 7.95 (s, 1H), 8.25 (d, 1H).

### Example 49

### 4-[[1-[[3-(1,1-dimethylethyl)phenyl]sulfonyl]-5-(trifluoromethyl)-1H-indol-2-yl]methyl]benzoic acid

Working similarly to example 2, starting from the ester of example 48, the expected compound was obtained as a white solid (yield=83%). M.p.=128° C.

### Example 49a

### 4-[[1-[[3-(1,1-dimethylethyl)phenyl]sulfonyl]-5-(trifluoromethyl)-1H-indol-2-yl]methyl]benzoic acid; sodium salt

15.5 mg (0.39 mM) of sodium hydroxide was added to a solution of 200 mg (0.39 mM) of 4-[[1-[[3-(1,1-dimethylethyl)phenyl]sulfonyl]-6-(trifluoro methyl)-1H-indol-2-yl]methyl]-benzoic acid in 10 mL of tetrahydrofuran. The reaction mixture was stirred overnight at room temperature, then evaporated under vacuum to obtain 195 mg of the sodium salt of 4-[[1-[[3-(1,1-dimethylethyl)phenyl]sulfonyl]-6-(trifluoromethyl)-1H-indol-2-yl]methyl]benzoic acid as a white solid (yield=94%).

¹H NMR (DMSO, 400 MHz) δ=1.18 (s, 9H), 4.37 (s, 2H), 6.39 (s, 1H), 7.10 (d, 2H), 7.48 (t, 1H), 7.62 (m, 2H), 7.73 (m, 2H), 7.80 (d, 2H), 7.91 (d, 1H), 8.27 (d, 1H).

### Example 49b

### 4-[[1-[[3-(1,1-dimethylethyl)phenyl]sulfonyl]-5-(trifluoromethyl)-1H-indol-2-yl]methyl]benzoic acid; tris(hydroxymethyl)aminomethane salt

47 mg (0.39 mM) of tris(hydroxymethyl)aminomethane and 2 mL of water were added to a solution of 200 mg (0.39 mM) of 4-[[1-[[3-(1,1-dimethylethyl)phenyl]sulfonyl]-6-(trifluoromethyl)-1H-indol-2-yl]methyl]benzoic acid in 10 mL of tetrahydrofuran. The reaction mixture was stirred overnight at room temperature, then evaporated under vacuum to obtain 110 mg of the tris(hydroxymethyl)aminomethane salt of 4-[[1-[[3-(1,1-dimethylethyl)phenyl]sulfonyl]-6-(trifluoromethyl)-1H-indol-2-yl]methyl]benzoic acid as a white solid (yield=45%).

¹H NMR (DMSO, 400 MHz) δ=1.17 (s, 9H), 4.45 (s, 2H), 6.49 (s, 1H), 7.23 (d, 2H), 7.48 (t, 1H), 7.62 (m, 2H), 7.72 (m, 2H), 7.84 (d, 2H), 7.92 (d, 1H), 8.25 (d, 1H).

### Example 49c

### 4-[[1-[[3-(1,1-dimethylethyl)phenyl]sulfonyl]-5-(trifluoromethyl)-1H-indol-2-yl]methyl]benzoic acid; piperazine salt

15 mg (0.17 mM) of piperazine was added to a solution of 90 mg (0.17 mM) of 4-[[1-[[3-(1,1-dimethylethyl)phenyl]sulfonyl]-6-(trifluoromethyl)-1H-indol-2-yl]methyl]benzoic acid in 10 mL of tetrahydrofuran. The reaction mixture was stirred for 1.5 h at room temperature, then evaporated under vacuum. The residue was then washed successively with petroleum ether, then with diethyl ether to obtain 8 mg of the piperazine salt of 4-[[1-[[3-(1,1-dimethylethyl)phenyl]sulfonyl]-6-(trifluoromethyl)-1H-indol-2-yl]methyl]benzoic acid as a white oil (yield=4%).

¹H NMR (DMSO, 400 MHz) δ=1.17 (s, 9H), 2.70 (s, 4); 4.45 (s, 2H), 6.50 (s, 1H), 7.24 (d, 2H), 7.47 (t, 1H), 7.63 (m, 2H), 7.73 (m, 2H), 7.84 (d, 2H), 7.93 (d, 1H), 8.27 (d, 1H).

### Preparation XVI

### N-(4-chloro-2-iodo-phenyl)-3-(1-methylethyl)-benzenesulfonamide

Working similarly to preparation X, starting from 4-chloro-2-iodo-aniline and 3-(1-methylethyl)benzenesulfonyl chloride, the expected compound was obtained as a beige solid (yield=51%).

¹H NMR (DMSOd₆, 250 MHz) δ=1.17 (d, 6H), 2.95 (sept, 1H), 7.05 (d, 1H), 7.46 (m, 5H), 7.88 (d, 1H), 9.76 (s broad, 1H).

### Example 50

### 4-[[5-chloro-1-[[3-(1-methylethyl)phenyl]sulfonyl]-1H-indol-2-yl]methyl]benzoic acid, methyl ester

Working similarly to example 48, starting from the compound obtained in preparation XVI, the expected compound was obtained as a beige solid (yield=19%).

¹H NMR (DMSOd₆, 250 MHz) δ=1.10 (d, 6H), 2.87 (sept, 1H), 3.85 (s, 3H), 4.50 (s, 2H), 6.46 (s, 1H), 7.47 (m, 8H), 7.91 (d, 2H), 8.03 (d, 1H).

### Example 51

### 4-[[5-chloro-1-[[3-(1-methylethyl)phenyl]sulfonyl]-1H-indol-2-yl]-methyl]benzoic acid

Working similarly to example 2, starting from the compound of example 50, the expected compound was obtained as a beige solid (yield=29%). M.p.=181° C.

### Preparation XVII

### N-(2-iodo-5-(trifluoromethyl)-phenyl)-3-(1,1-dimethylethyl)-benzene-sulfonamide

Working similarly to preparation X, starting from 2-iodo-5-(trifluoromethyl)aniline and 3-(1,1-dimethylethyl)benzenesulfonyl chloride, the expected compound was obtained as a white solid (yield=74%). M.p.=134° C.

### Example 52

### 4-[[1-[[3-(1,1-dimethylethyl)phenyl]sulfonyl]-6-(trifluoromethyl)-1H-indol-2-yl]methyl]benzoic acid, methyl ester

Working similarly to example 48, starting from the compound obtained in preparation XVII, the expected compound was obtained as a yellow oil (yield=42%).

¹H NMR (DMSOd₆, 250 MHz) δ=1.19 (s, 9H), 3.85 (s, 3H), 4.55 (s, 2H), 6.60 (s, 1H), 7.39 (d, 2H), 7.50 (d, 1H), 7.59 (m, 2H), 7.73 (m, 3H), 7.92 (d, 2H), 8.29 (s, 1H).

### Example 53

### 4-[[1-[[3-(1,1-dimethylethyl)phenyl]sulfonyl]-6-(trifluoromethyl)-1H-indol-2-yl]methyl]benzoic acid

Working similarly to example 2, starting from the compound of example 52, the expected compound was obtained as a white solid (yield=17%). M.p.=199° C.

### Preparation XVIII

### N-(3-chloro-2-iodophenyl)-3,4-dihydro-4-methyl-2H-1,4-benzoxazine-6-sulfonamide

Working similarly to preparation X, starting from 2-iodo-3-chloroaniline and 3,4-dihydro-4-methyl-2H-1,4-benzoxazine-6-sulfonyl chloride, the expected compound was obtained as a white solid (yield=76%).

¹H NMR (DMSOd₆, 300 MHz) δ=2.79 (s, 3H), 3.29 (m, 2H), 4.28 (m, 2H), 6.79 (d, 1H), 6.95 (m, 3H), 7.30 (t, 1H), 7.40 (d, 1H), 9.56 (s, 1H).

### Example 54

### 4-[[4-chloro-1-[(3,4-dihydro-4-methyl-2H-1,4-benzoxazin-6-yl)sulfonyl]-1H-indol-2-yl]methyl]benzoic acid, methyl ester

Working similarly to example 48, starting from the compound obtained in preparation XVIII, the expected compound was obtained as a white solid (yield=18%).

¹H NMR (DMSOd₆, 250 MHz) δ=2.73 (s, 3H), 3.22 (m, 2H), 3.85 (s, 3H), 4.22 (m, 2H), 4.52 (s, 2H), 6.51 (s, 1H), 6.72 (d, 1H), 6.82 (d, 1H), 6.98 (dd, 1H), 7.34 (m, 4H), 7.90 (d, 2H), 8.05 (m, 1H).

### Example 55

### 4-[[4-chloro-1-[(3,4-dihydro-4-methyl-2H-1,4-benzoxazin-6-yl)sulfonyl]-1H-indol-2-yl]methyl]benzoic acid

Working similarly to example 2, starting from the compound of example 54, the expected compound was obtained as a white solid (yield=21%). M.p.=236° C.

### Example 56

### 4-[[1-[[3-(1,1-dimethylethyl)phenyl]sulfonyl]-5-trifluoromethyl-1H-indol-2-ylmethyl]-2-hydroxybenzoic acid

Working similarly to example 2, starting from the compound of example 11, 4-[[1-[[3-(1,1-dimethylethyl)phenyl]sulfonyl]-5-trifluoromethyl-1H-indol-2-ylmethyl]-2-methoxybenzoic acid was obtained.

0.73 mL (0.73 mM) of a 1M solution of boron tribromide (BBr₃) in dichloromethane was added dropwise to a solution of 200 mg (0.37 mM) of this compound in 10 mL of dichloromethane cooled to −78° C. The reaction mixture was stirred for 5 hours at −78° C., then hydrolysed with 20 mL of water. After decanting and extracting with dichloromethane, the combined organic phases were dried over magnesium sulfate and concentrated under reduced pressure. As the reaction was incomplete, the residue was put back in solution in 10 mL of dichloromethane at −78° C., and 0.73 mL (0.73 mM) of a 1M solution of BBr₃ in dichloromethane was added dropwise. The reaction mixture was stirred for 3 h at −78° C., then hydrolysed with water. After extracting twice with dichloromethane, the combined organic phases were dried over magnesium sulfate and concentrated under reduced pressure. The residue obtained was purified by preparative liquid chromatography, eluting with H₂O/CH₃CN/0.1% TFA mixture. The fractions containing the expected product were combined and concentrated to dryness under reduced pressure to give 85 mg of 4-[[1-[[3-(1,1-dimethylethyl)phenyl]sulfonyl]-5-trifluoromethyl-1H-indol-2-ylmethyl]-2-hydroxybenzoic acid as a white solid (yield=44%). M.p.=129° C.

### Example 57

### 4-[1-(3-bromo-benzenesulfonyl)-5-trifluoromethyl-1H-indol-2-ylmethyl]-benzoic acid, methyl ester

17 mg (0.71 mM) of sodium hydride (60% dispersion in oil) was added to a solution of 83 mg (0.25 mM) of 4-(5-trifluoromethyl-1H-indol-2-ylmethyl)-benzoic acid methyl ester obtained in preparation VII in 2 mL of DMF cooled to 0° C. After stirring for 5 minutes at 0° C., 140 mg (0.55 mM) of 3-bromobenzene sulfonyl chloride was added dropwise. The reaction mixture was stirred for 15 minutes at 0° C., then hydrolysed with 100 mL of a 10% aqueous solution of NH₄Cl, then extracted 3 times with 50 mL of ethyl acetate. The combined organic phases were dried over magnesium sulfate and concentrated under reduced pressure. The residue obtained was purified by silica gel chromatography, eluting with a cyclohexane/ethyl acetate mixture (90/10; v/v). The fractions containing the expected product were combined and concentrated to dryness under reduced pressure to give 110 mg of 4-[1-(3-bromo-benzenesulfonyl)-5-trifluoromethyl-1H-indol-2-ylmethyl]-benzoic acid methyl ester as an orange solid (yield=80%).

¹H NMR (DMSOd₆, 300 MHz) δ=3.85 (s, 3H), 4.55 (s, 2H), 6.73 (s, 1H), 7.36 (d, 2H), 7.48 (t, 1H), 7.67 (m, 1H), 7.74 (t, 1H), 7.82 (m, 1H), 7.87 (m, 1H), 7.89 (d, 2H), 8.01 (s, 1H), 8.24 (d, 1H).

### Example 58

### 4-[1-(3-cyclopropyl-benzenesulfonyl)-5-trifluoromethyl-1H-indol-2-ylmethyl]-benzoic acid methyl ester

161 mg (0.76 mM) of tribasic potassium phosphate, 5.58 mg (0.02 mM) of tricyclohexyl phosphine, 2.24 mg (0.01 mM) of palladium acetate and 0.06 mL of water were added to a solution of 110 mg (0.20 mM) of 4-[1-(3-bromo-benzenesulfonyl)-5-trifluoromethyl-1H-indol-2-ylmethyl]-benzoic acid methyl ester obtained in example 57 and 24 mg (0.28 mM) of cyclopropyl boronic acid in 1.38 mL of toluene. The reaction mixture was heated for 1 hour at 100° C. in microwave equipment, then diluted in water and extracted twice with ethyl acetate. The combined organic phases were dried over magnesium sulfate and evaporated under reduced pressure. The reaction was then started again in the same conditions as previously (same amount of reactant). The reaction mixture was heated for 1 hour at 100° C. in microwave equipment then diluted in water and extracted twice with ethyl acetate. The combined organic phases were dried over magnesium sulfate and evaporated under reduced pressure. The residue was purified by silica gel chromatography, eluting with a cyclohexane/ethyl acetate mixture (90/10; v/v). The fractions containing the expected product were combined and concentrated to dryness under reduced pressure to give 54 mg of 4-[1-(3-cyclopropyl-benenesulfonyl)-5-trifluoromethyl-1H-indol-2-ylmethyl]-benzoic acid methyl ester as a yellow solid (yield=53%).

¹H NMR (DMSOd₆, 300 MHz) δ=0.60 (m, 2H), 0.96 (m, 2H), 1.92 (m, 1H), 3.85 (s, 3H), 4.55 (s, 2H), 6.64 (s, 1H), 7.31 (d, 1H), 7.38 (m, 4H), 7.54 (d, 1H), 7.65 (d, 1H), 7.90 (d, 2H), 7.97 (s, 1H), 8.23 (d, 1H).

### Example 59

### 4-[1-(3-cyclopropyl-benzenesulfonyl)-5-trifluoromethyl-1H-indol-2-ylmethyl]-benzoic acid

Working similarly to example 2, starting from the ester of example 58, the expected compound was obtained as a white solid (yield=79%). M.p.=147° C.

### Preparation XIX

### 1,2-dimethyl-3-(piperidine-1-sulfonyl)-3H-imidazol-1-ium trifluoromethanesulfonate

133 μL (1.13 mM) of methyl trifluoromethanesulfonate was added to a solution of 0.25 g (1.07 mM) of 1-(2-methyl-imidazole-1-sulfonyl)-piperidine in 6 mL of dichloromethane cooled to −5° C. The reaction mixture was stirred for 1 hour at 0° C., then concentrated under vacuum. 400 mg of 1,2-dimethyl-3-(piperidine-1-sulfonyl)-3H-imidazol-1-ium was obtained as a white powder (yield=96%). M.p.=169° C.

### Preparation XX

### N-(4-chloro-2-iodophenyl)-1-piperidinesulfonamide

0.23 g (0.90 mM) of 4-chloro-2-iodoaniline and 0.380 g (0.97 mM) of 1,2-dimethyl-3-(piperidine-1-sulfonyl)-3H-imidazol-1-ium trifluoromethanesulfonate obtained in preparation XIX in solution in 3.5 mL of acetonitrile were heated for 30 minutes at 150° C. in microwave equipment. The reaction mixture was diluted with ethyl acetate and washed with water. The aqueous phase was extracted three times with ethyl acetate and the organic phases were combined and washed with a saturated aqueous solution of sodium chloride. The organic phase was dried over magnesium sulfate and evaporated under reduced pressure. The residue was purified by silica gel chromatography, eluting with a cyclohexane/ethyl acetate mixture (95/5; v/v). The residue was purified again by silica gel chromatography, eluting with toluene. The fractions containing the expected product were combined and concentrated to dryness under reduced pressure to give 130 mg of N-(4-chloro-2-iodophenyl)-1-piperidinesulfonamide as a pink oil (yield=35%).

¹H NMR (DMSOd₆, 300 MHz) δ=1.47 (m, 6H), 3.13 (m, 4H), 7.45 (m, 2H), 7.92 (d, 1H), 9.13 (s, 1H).

### Example 60

### 4-[hydroxy[5-chloro-1-(1-piperidinylsulfonyl)-1H-indol-2-yl]methyl]-benzoic acid, methyl ester

Working similarly to example 30, starting from the compound obtained in preparation XX, the expected compound was obtained as a yellow solid (yield=88%).

¹H NMR (DMSOd₆, 500 MHz) δ=1.34 (m, 6H), 3.11 (m, 4H), 3.84 (s, 3H), 6.28 (s broad, 1H), 6.32 (s broad, 1H), 6.72 (s, 1H), 7.31 (dd, 1H), 7.47 (d, 2H), 7.71 (d, 1H), 7.87 (d, 1H), 7.93 (d, 2H).

### Example 61

### 4-[[5-chloro-1-(1-piperidinylsulfonyl)-1H-indol-2-yl]methyl]benzoic acid methyl ester

Working similarly to example 31, starting from the compound of example 60, the expected compound was obtained as a white solid (yield=17%). M.p.=133° C.

### Preparation XXI

### 3-(1-hydroxy-2-propynyl)-benzoic acid

Under an argon atmosphere, 23 mL (0.0115 mol) of ethynyl magnesium bromide was added to a solution of 0.7 g (0.0043 mol) of 3-formyl-benzoic acid methyl ester in 25 mL of tetrahydrofuran and the reaction mixture was stirred overnight at room temperature. The reaction mixture was diluted with a saturated aqueous solution of NH₄Cl and washed 3 times with ethyl acetate. The aqueous phase was then acidified with 1N hydrochloric acid (HCl), then extracted 3 times with dichloromethane. The combined chlorinated organic phases were dried over magnesium sulfate and evaporated under reduced pressure. 563 mg of 3-(1-hydroxy-2-propynyl)-benzoic acid was thus obtained as a white solid (yield=69%).

¹H NMR (DMSOd₆, 250 MHz) δ=3.53 (d, 1H), 5.45 (m, 1H), 6.17 (d, 1H), 7.49 (t, 1H), 7.69 (dt, 1H), 7.86 (dt, 1H), 8.07 (t, 1H), 12.98 (s broad, 1H).

### Example 62

### 3-[[5-chloro-1-[[4-(1-methylethyl)phenyl]sulfonyl]-1H-indol-2-yl]-hydroxymethyl]benzoic acid

Working similarly to example 30, using the 3-(1-hydroxy-2-propynyl)-benzoic acid obtained in preparation XXI and the compound obtained in preparation XIV, the expected product was obtained as a white solid (yield=65%). M.p.=97° C.

### Example 63

### 3-[[5-chloro-1-[[4-(1-methylethyl)phenyl]sulfonyl]-1H-indol-2-yl]methyl]benzoic acid

Working similarly to example 31, starting from the compound of example 62, the expected compound was obtained as a yellow solid (yield=14%). M.p.=170° C.

### Example 64

### 6-[[1-[[3-(1,1-dimethylethyl)phenyl]sulfonyl]-5-(trifluoromethyl)-1H-indol-2-yl]hydroxymethyl]-3-pyridinecarboxylic acid, methyl ester

Under an argon atmosphere, 2.46 mL (3.93 mM) of a solution of n-butyllithium (c=1.6 M in hexane) was added slowly to a solution of 1 g (2.62 mM) of 1-(3-tert-butyl-benzenesulfonyl)-5-trifluoromethyl-1H-indole (preparation III) in 10 mL of tetrahydrofuran cooled to −8° C. The reaction mixture was stirred for 1.5 h at 0° C., then added dropwise at −70° C. to a solution of 433 mg (2.62 mM) of methyl 6-formylnicotinate in 20 mL of tetrahydrofuran. The reaction mixture was stirred for 2 hours at −70° C., then diluted with water and extracted with ethyl acetate. The organic phase was dried over magnesium sulfate and then evaporated under reduced pressure. The residue was purified by silica gel chromatography, eluting with a cyclohexane/ethyl acetate mixture (90/10; v/v), then with a dichloromethane/ethyl acetate mixture (80/20; v/v). The fractions containing the expected product were combined and concentrated to dryness under reduced pressure to give 522 mg of 6-{[1-[[3-(1,1-dimethylethyl)phenyl]sulfonyl]-5-trifluoromethyl-1H-indol-2-yl]hydroxymethyl}nicotinic acid methyl ester as a brown paste (yield=36%).

¹H NMR (DMSOd₆, 300 MHz) δ=1.22 (s, 9H), 3.90 (s, 3H), 6.54 (s, 1H), 6.60 (d, 1H), 6.74 (d, 1H), 7.51 (t, 1H), 7.66 (dd, 1H), 7.73 (m, 1H), 7.80 (d, 1H), 7.81 (m, 1H), 7.97 (m, 2H), 8.25 (d, 1H), 8.39 (dd, 1H), 8.98 (dd, 1H).

### Example 65

### 6-[[1-[[3-(1,1-dimethylethyl)phenyl]sulfonyl]-5-(trifluoromethyl)-1H-indol-2-yl]methyl]-3-pyridinecarboxylic acid, methyl ester

27.87 μL (0.38 mM) of SOCl₂ was added to a solution of 140 mg (0.26 mM) of the acid obtained in example 64 in 1 mL of dichloromethane cooled to 5° C., then the reaction mixture was stirred for 4 hours at room temperature. The solution was then cooled to 5° C., diluted with water and the pH of this solution was adjusted to 8 by addition of a saturated aqueous solution of sodium hydrogen carbonate (NaHCO₃). After extraction with dichloromethane, the organic phase was dried over magnesium sulfate and evaporated under reduced pressure. The crude product was taken up in 1 mL of acetic acid and 83.75 mg (1.28 mM) of zinc was added. The reaction mixture was stirred for 7.5 h at room temperature, and for 1.5 h at reflux temperature. After filtration to remove the zinc and evaporate the solvents, the residue was taken up in dichloromethane and washed with water. The organic phase was dried over magnesium sulfate and evaporated under reduced pressure. The residue was purified by silica gel chromatography, eluting with a cyclohexane/ethyl acetate mixture (95/5; v/v) and the fractions containing the expected product were combined and concentrated to dryness under reduced pressure to give 46 mg as an orange paste (yield=34%).

¹H NMR (DMSOd₆, 400 MHz) δ=1.18 (s, 9H), 3.88 (s, 3H), 4.70 (s, 2H), 6.68 (s, 1H), 7.42 (d, 1H), 7.48 (t, 1H), 7.63 (dm, 1H), 7.67 (dd, 1H), 7.69 (t, 1H), 7.72 (dm, 1H), 7.97 (s, 1H), 8.23 (dd, 1H), 8.27 (d, 1H), 8.95 (dd, 1H).

### Example 66

### 6-[[1-[[3-(1,1-dimethylated)phenyl]sulfonyl]-5-(trifluoromethyl)-1H-indol-2-yl]hydroxymethyl]-3-pyridinecarboxylic acid

Working similarly to example 2, starting from the ester of example 65, the expected compound was obtained as an orange solid (yield=44%). M.p.=200° C.

### Preparation XXII

### α-(4-bromo-2-fluorophenyl)-1-[[3-(1,1-dimethylethyl)phenyl]sulfonyl]-5-(trifluoromethyl)-1H-indole-2-methanol

Working similarly to example 64 starting from the compound prepared in preparation III and 4-bromo-2-fluoro-benzaldehyde, the expected compound was obtained as a beige solid (yield=39%).

¹H NMR (DMSOd₆, 300 MHz) δ=1.20 (s, 9H), 6.52 (s, 1H), 6.62 (s, 1H), 6.74 (s, 1H), 7.29 (t, 1H), 7.42 (dd, 1H), 7.49 (t, 1H), 7.57 (dd, 1H), 7.67 (m, 1H), 7.70 (m, 1H), 7.74 (dm, 1H), 7.88 (t, 1H), 8.02 (s, 1H), 8.26 (d, 1H).

### Preparation XXIII

### 2-[(4-bromo-2-fluoro-phenyl)methyl]-1-[[3-(1,1-dimethylethyl)phenyl]sulfonyl]-5-(trifluoromethyl)-1H-indole

Working similarly to example 31, starting from the compound prepared in preparation XXII, the expected compound was obtained as a colourless oil (yield=71%).

¹H NMR (DMSOd₆, 300 MHz) δ=1.19 (s, 9H), 4.41 (s, 2H), 6.41 (s, 1H), 7.23 (t, 1H), 7.40 (dd, 1H), 7.52 (t, 1H), 7.58 (dd, 1H), 7.72 (m, 4H), 7.93 (s, 1H), 8.30 (d, 1H).

### Example 67

### 4-[[1-[[3-(1,1-dimethylethyl)phenyl]sulfonyl]-5-trifluoromethyl-1H-indol-2-yl]methyl]-3-fluorobenzoic acid

A mixture of 136 mg (0.24 mM) of 2-[(4-bromo-2-fluorophenyl)methyl]-1-[[3-(1,1-dimethylethyl)phenyl]sulfonyl]-5-trifluoromethyl-1H-indole (preparation XXIII), 5.37 mg (0.02 mM) of palladium acetate, 6.94 mg (0.02 mM) of triterbutylphosphonium tetrafluoroborate, 94.75 mg (0.36 mM) of molybdenum hexacarbonyl, 38.04 mg (0.36 mM) of sodium carbonate in 1.63 mL of DME and 0.54 mL of water was heated for 1 hour at 120° C. in microwave equipment. The reaction mixture was filtered on paper and the filtrate was evaporated. The residue obtained was purified by preparative liquid chromatography, eluting with H₂O/CH₃CN/0.1% TFA mixture. The fractions containing the expected product were combined and concentrated to dryness under reduced pressure to give 101 mg of 4-[[1-[[3-(1,1-dimethylethyl)phenyl]sulfonyl]-5-trifluoro methyl-1H-indol-2-yl]methyl]-3-fluorobenzoic acid as a white solid (yield=79%). M.p.=177° C.

### Example 68

### 2-hydroxy-4-[[1-[[4-(1-methylethyl)phenyl]sulfonyl]-5-(trifluoromethyl)-1H-indol-2-yl]methyl]-benzoic acid

Working similarly to example 56, starting from the compound of example 3, the expected compound was obtained as a white solid (yield=22%). M.p.=150° C.

### Preparation XXIV

### [4-methyl-3,4-dihydro-2H-benzo[1,4]oxazin-6-yl]-sulfonic acid (2-iodo-4-chloromethyl-phenyl)-amide

Working similarly to preparation X starting from 2-iodo-4-chloro-phenylamine and 2,3-dihydro-benzo[1,4]dioxin-6-sulfonyl chloride, the expected product was obtained as a white solid (yield 74%). M.p.=109-110° C.

### Preparation XXV

### [1-[[3-(1,1-dimethylethyl)-phenyl]sulfonyl]-5-trifluoromethyl-1H-indol-2-yl]-methanol

A mixture of 18 g (37.25 mM) of 3-(1,1-dimethylethyl)-N-[2-iodo-4-(trifluoromethyl)phenyl]-benzenesulfonamide, 2.64 mL (44.7 mM) of prop-2-yn-1-ol, 0.52 g (0.74 mM) of bis-triphenylphosphine palladium (II) chloride, 0.35 g (1.86 mM) of copper iodide, 100 mL of diethylamine and 100 mL of dimethylformamide was heated for one hour under reflux. The reaction mixture was diluted with ethyl acetate and washed successively with water and then with a saturated NaCl aqueous solution. The organic phase was dried over magnesium sulfate and concentrated under reduced pressure to give 14.7 g of [1-[[3-(1,1-dimethylethyl)phenyl]sulfonyl]-5-trifluoromethyl-1H-indol-2-yl]-methanol as a brown oil (yield=96%).

1H NMR (300 MHz, DMSOd₆)

δ=8.26 (d, 1H), 8.00 (s, 1H), 7.93 (m, 1H), 7.80 (d, 1H), 7.75 (d, 1H), 7.64 (m, 1H), 7.54 (t, 1H), 6.91 (s, 1H), 5.68 (t, 1H), 4.88 (d, 2H), 1.20 (s, 9H).

### Preparation XXVI

### [1-[[4-(1-methylethyl)phenyl]sulfonyl]-5-(trifluoromethyl)-1H-indol-2-yl]-methanol

Working similarly to preparation XXV starting from the compound obtained according to preparation X, the expected product was obtained as a beige solid (yield 55%). M.p.=112° C.

### Preparation XXVII

### [1-[[4-methyl-3,4-dihydro-2H-benzo[1,4]oxazin-6-yl]-sulfonyl]-5-trifluoro-methyl-1H-indol-2-yl]-methanol

Working similarly to preparation XXV starting from the compound obtained according to preparation XII, the expected product was obtained as an orange solid (yield 91%).

¹H NMR (300 MHz, DMSOd₆) δ=8.23 (d, 1H), 7.99 (s, 1H), 7.61 (dd, 1H), 7.17 (m, 2H), 6.88 (s, 1H), 6.80 (d, 1H), 5.66 (t, 1H), 4.88 (d, 2H), 4.23 (m, 2H), 3.25 (m, 2H), 2.79 (s, 3H).

### Preparation XXVIII

### [5-chloro-1-[[2,3-dihydro-benzo[1,4]dioxin-6-yl]-sulfonyl]-1H-indol-2-yl]-methanol

Working similarly to preparation XXV starting from the compound obtained according to preparation XXIV, the expected product was obtained as an orange solid (yield 86%).

¹H NMR (300 MHz, DMSOd₆) δ=7.99 (d, 1H), 7.65 (d, 1H), 7.42 (m, 2H), 7.32 (dd, 1H), 7.02 (d, 1H), 6.74 (s, 1H), 5.60 (t, 1H), 4.83 (d, 2H), 4.27 (m, 4H).

### Preparation XXIX

### 2-bromomethyl-1-[[3-(1,1-dimethylethyl)-phenyl]sulfonyl]-5-trifluoromethyl-1H-indole

3.65 mL (38.9 mM) of phosphorus tribromide was added dropwise to a solution of 4 g (9.72 mM) of [1-[[3-(1,1-dimethylethyl)-phenyl]sulfonyl]-5-trifluoromethyl-1H-indol-2-yl]methanol (preparation XXV) in 15 mL of dichloromethane cooled to 0° C. The reaction mixture was stirred for 1 hour at room temperature. 20 mL of ethanol was then added slowly and then the reaction mixture was poured onto ice. After extracting twice with dichloromethane, the combined organic phases were dried over MgSO₄ and concentrated to dryness under reduced pressure. The residue was purified by silica gel chromatography, eluting with a cyclohexane/ethyl acetate mixture (95/5; v/v) then progressively with a cyclohexane/ethyl acetate mixture (90/10; v/v). The fractions containing the expected product were combined and concentrated to dryness under reduced pressure to give 4 g of 2-bromomethyl-1-[[3-(1,1-dimethylethyl)-phenyl]sulfonyl]-5-trifluoromethyl-1H-indole as a white solid (yield=87%). M.p.=80° C.

### Preparation XXX

### 2-(bromomethyl)-1-[[4-(1-methylethyl)phenyl]sulfonyl]-5-(trifluoromethyl)-1H-indole

Working similarly to preparation XXIX starting from the compound obtained according to preparation XXVI, the expected product was obtained as a white solid (yield 78%). M.p.=100° C.

### Preparation XXXI

### 6[-[2-bromomethyl-5-trifluoromethyl-indol-1-yl]-sulfonyl]-4-methyl-3,4-dihydro-2H-benzo[1,4]oxazine

Working similarly to preparation XXIX starting from the compound obtained according to preparation XXVII, the expected product was obtained as an orange oil (yield 58%).

¹H NMR (300 MHz, DMSOd₆) δ=8.21 (d, 1H), 8.05 (s, 1H), 7.69 (dd, 1H), 7.21 (m, 2H), 7.08 (d, 1H), 6.80 (d, 1H), 5.22 (s, 1H), 4.23 (m, 2H), 3.24 (m, 2H), 2.80 (s, 3H).

### Preparation XXXII

### 2-bromomethyl-5-chloro-1-[[2,3-dihydro-benzo[1,4]dioxin-6-yl]-sulfonyl]-1H-indole

Working similarly to preparation XXIX starting from the compound obtained according to preparation XXVIII, the expected product was obtained as a white solid (yield 81%).

¹H NMR (300 MHz, DMSOd₆) δ=7.99 (d, 1H), 7.70 (d, 1H), 7.43 (m, 3H), 7.08 (s, 1H), 7.01 (d, 1H), 5.16 (s, 2H), 4.26 (m, 4H).

### Example 69

### 5-[[1-[[3-(1,1-dimethylethyl)-phenyl]sulfonyl]-5-trifluoromethyl-1H-indol-2-yl]methyl]-furan-2-carboxylic acid

78.9 mg (0.51 mM) of 5-(dihydroxyboryl)-2-furoic acid, 34.4 mg (0.04 mM) of Pd(dppf)Cl₂.CH₂Cl₂ complex and 165.8 mg (1.2 mM) of potassium carbonate were added successively to a solution of 200 mg (0.42 mM) of 2-bromomethyl-1-(3-(1,1-dimethylethyl)-benzenesulfonyl)-5-trifluoromethyl-1H-indole (preparation XXIX) in 4 mL of ethanol and 1 mL of 1,4-dioxane. The reaction mixture was heated for 20 minutes by microwave at 120° C., then diluted with ethyl acetate and washed successively with water and then with saturated NaCl aqueous solution. The organic phase was dried over magnesium sulfate and concentrated under reduced pressure. The residue obtained was purified by preparative LC-UV (Sunfire C18), eluting with H₂O/CH₃CN/0.1% TFA mixture. The fractions containing the expected product were combined and concentrated to dryness under reduced pressure to give 15 mg of 5-[1-(3-(1,1-dimethylethyl)-benzenesulfonyl)-5-trifluoromethyl-1H-indol-2-ylmethyl]-furan-2-carboxylic acid as an orange oil (yield=7%).

¹H NMR (400 MHz, DMSOd₆) δ=12.9 (sl, 1H), 8.27 (d, 1H), 7.99 (s, 1H), 7.70 (m, 4H), 7.50 (t, 1H), 7.14 (m, 1H), 6.69 (s, 1H), 6.40 (m, 1H), 4.58 (s, 2H), 1.18 (s, 9H).

### Example 70

### 4-[[1-[[3-(1,1-dimethylethyl)-phenyl]sulfonyl]-5-trifluoromethyl-1H-indol-2-yl]methyl]-thiophene-2-carboxylic acid

Working similarly to example 69 starting from the compound obtained according to preparation XXIX and 4-(dihydroxyboryl)-2-thiophene carboxylic acid, the expected product was obtained as an orange oil (yield 30%).

¹H NMR (400 MHz, DMSOd₆) δ=13.10 (sl, 1H), 8.26 (d, 1H), 7.95 (s, 1H), 7.72 (d, 1H), 7.68 (t, 1H), 7.73 (m, 3H), 7.54 (m, 1H), 7.48 (t, 1H), 6.62 (s, 1H), 4.44 (s, 2H), 1.17 (s, 9H).

### Example 71

### 5-[[1-[[4-(1-methylethyl)-phenyl]sulfonyl]-5-trifluoromethyl-1H-indol-2-yl]methyl]-thiophene-2-carboxylic acid

Working similarly to example 69 starting from the compound obtained according to preparation XXX and 5-(dihydroxyboryl)-2-thiophene carboxylic acid, the expected product was obtained as a beige solid (yield 6%). M.p.=199-216° C.

### Example 72

### 4-[[1-[[4-(1-methylethyl)-phenyl]sulfonyl]-5-trifluoromethyl-1H-indol-2-yl]methyl]-thiophene-2-carboxylic acid

Working similarly to example 69 starting from the compound obtained according to preparation XXX and 4-(dihydroxyboryl)-2-thiophene carboxylic acid, the expected product was obtained as a brown solid (yield 39%).

¹H NMR (400 MHz, DMSOd₆) δ=13.03 (s, 1H), 8.25 (d, 1H), 7.97 (s, 1H), 7.75 (d, 2H), 7.64 (m, 2H), 7.55 (s, 1H), 7.47 (d, 1H), 6.62 (s, 1H), 4.46 (s, 2H), 2.93 (m, 1H), 1.14 (d, 6H).

### Example 73

### 5-[[1-[[4-methyl-3,4-dihydro-2H-benzo[1,4]oxazin-6-yl]-sulfonyl]-5-trifluoromethyl-1H-indol-2-yl]methyl]-thiophene-2-carboxylic acid

Working similarly to example 69 starting from the compound obtained according to preparation XXXI and 5-(dihydroxyboryl)-2-thiophene carboxylic acid, the expected product was obtained as a beige solid (yield 4%). M.p.=120-144° C.

### Example 74

### 4-[[1-[(4-methyl-3,4-dihydro-2H-benzo[1,4]oxazin-6-yl)-sulfonyl]-5-trifluoromethyl-1H-indol-2-yl]methyl]-thiophene-2-carboxylic acid

Working similarly to example 69 starting from the compound obtained according to preparation XXXI and 4-(dihydroxyboryl)-2-thiophene carboxylic acid, the expected product was obtained as a brown solid (yield 15%).

¹H NMR (500 MHz, DMSOd₆) δ=13.10 (s, 1H), 8.26 (d, 1H), 7.96 (s, 1H), 7.62 (m, 2H), 7.55 (s, 1H), 7.02 (dd, 1H), 6.89 (d, 1H), 6.74 (d, 1H), 6.60 (s, 1H), 4.44 (s, 2H), 4.23 (t, 2H), 3.24 (t, 2H), 2.77 (s, 3H).

### Example 75

### 5-[[1-[(4-methyl-3,4-dihydro-2H-benzo[1,4]oxazin-6-yl)-sulfonyl]-5-trifluoromethyl-1H-indol-2-yl]methyl]-furan-2-carboxylic acid

Working similarly to example 69 starting from the compound obtained according to preparation XXXI and 5-(dihydroxyboryl)-2-furoic acid, the expected product was obtained as a brown solid (yield 4%).

¹H NMR (400 MHz, DMSOd₆) δ=12.60 (s, 1H), 8.29 (d, 1H), 8.19 (s, 1H), 7.96 (s, 1H), 7.65 (dd, 1H), 7.05 (dd, 1H), 6.91 (d, 1H), 6.75 (d, 1H), 6.61 (s, 1H), 6.44 (s, 1H), 4.51 (s, 2H), 4.23 (t, 2H), 3.24 (t, 2H), 2.78 (s, 3H).

### Example 76

### 5-[[1-[[4-methyl-3,4-dihydro-2H-benzo[1,4]oxazin-6-yl)-sulfonyl]-5-trifluoromethyl-1H-indol-2-yl]methyl]-furan-3-carboxylic acid

Working similarly to example 69 starting from the compound obtained according to preparation XXXI and 5-(dihydroxyboryl)-3-furoic acid, the expected product was obtained as a black solid (yield 6%).

¹H NMR (500 MHz, DMSOd₆) δ=13.00 (s, 1H), 8.27 (d, 1H), 7.99 (s, 1H), 7.67 (dd, 1H), 7.17 (dd, 1H), 7.07 (dd, 1H), 6.92 (s, 1H), 6.78 (d, 1H), 6.63 (s, 1H), 6.40 (d, 1H), 4.56 (s, 2H), 4.23 (t, 2H), 3.24 (t, 2H), 2.78 (s, 3H).

### Example 77

### 5-[[5-chloro-1-[(2,3-dihydro-benzo[1,4]dioxin-6-yl)sulfonyl]-1H-indol-2-yl]methyl]-thiophene-2-carboxylic acid

Working similarly to example 69 starting from the compound obtained according to preparation XXXII and 5-(dihydroxyboryl)-2-thiophene carboxylic acid, the expected product was obtained as a white solid (yield=4%). M.p.=196° C.

### Example 78

### 4-[[5-chloro-1-[2,3-dihydro-benzo[1,4]dioxin-6-yl]-sulfonyl]-1H-indol-2-yl]methyl]-thiophene-2-carboxylic acid

Working similarly to example 69 starting from the compound obtained according to preparation XXXII and 4-(dihydroxyboryl)-2-thiophene carboxylic acid, the expected product was obtained as a brown solid (yield 24%).

¹H NMR (400 MHz, DMSOd₆) δ=13.01 (s, 1H), 8.03 (d, 1H), 7.55 (m, 3H), 7.33 (dd, 1H), 7.29 (dd, 1H), 7.12 (d, 1H), 6.94 (d, 1H), 6.48 (s, 1H), 4.38 (s, 2H), 4.26 (m, 4H).

### Example 79

### 5-[[5-chloro-1-[(2,3-dihydro-benzo[1,4]dioxin-6-yl)sulfonyl]-1H-indol-2-yl]methyl]-furan-2-carboxylic acid

Working similarly to example 69 starting from the compound obtained according to preparation XXXII and 5-(dihydroxyboryl)-2-furoic acid, the expected product was obtained as a beige solid (yield 12%).

¹H NMR (400 MHz, DMSOd₆) δ=12.95 (s, 1H), 8.02 (d, 1H), 6.64 (d, 1H), 7.33-7.39 (m, 2H), 7.22 (d, 1H), 7.16 (d, 1H), 7.01 (d, 1H), 6.51 (s, 1H), 6.40 (d, 1H), 4.53 (s, 2H), 4.26 (m, 4H).

### Preparation XXXIII

### 1-[[3-(1,1-dimethylethyl)-phenyl]sulfonyl]-3-methyl-5-trifluoromethyl-1H-indole

1.43 g (59.5 mM) of sodium hydride was added by portions to a solution of 7.9 g (39.6 mM) of 3-methyl-5-trifluoromethyl-1H-indole in 79 mL of dimethylformamide. The reaction mixture was stirred for 10 minutes at 0° C., then 10.15 g (43.63 mM) of 3-(1,1-dimethylethyl)benzenesulfonyl chloride was added slowly. After stirring for 1 hour, the mixture was hydrolysed with 500 mL of ice water and 100 mL of 1N hydrochloric acid and filtered on a Buchner filter. The solid was washed with water, then dried to give 14.9 g of 1-[[3-(1,1-dimethylethyl)-phenyl]sulfonyl]-3-methyl-5-trifluoro methyl-1H-indole as an orange solid (yield=95%). M.p.=90-108° C.

### Preparation XXXIV

### [4-formyl-1-methyl-1H-pyrrol-2-yl]-carboxylic acid (1,1-dimethylethyl)ester

A solution of 796 mg (5.20 mM) of [4-formyl-1-methyl-1H-pyrrol-2-yl]-carboxylic acid in 20 mL of toluene was heated to reflux and 9.97 ml (41.58 mM) of di-tert-butyl acetal N,N-dimethylformamide was added slowly (the mixture becomes homogeneous as addition proceeds). The reaction mixture was stirred for 2 hours at reflux temperature, then hydrolysed with water and extracted with ethyl acetate. The organic phase was then washed successively with a saturated aqueous solution of NaHCO₃ then of NaCl, dried over magnesium sulfate and evaporated under reduced pressure. The residue was purified by silica gel chromatography, eluting with a cyclohexane/ethyl acetate mixture (95/5; v/v) then progressively up to cyclohexane/ethyl acetate 60/40, (v/v). The fractions containing the expected product were combined and concentrated to dryness under reduced pressure to give 515 mg of 4-formyl-1-methyl-1H-pyrrole-2-carboxylic acid (1,1-dimethylethyl)ester as a beige solid (yield=47%). M.p.=92° C.

### Preparation XXXV

### 1-[[3-(1,1-dimethylethyl)-phenyl]sulfonyl]-5-trifluoromethyl-1H-indole

Working similarly to preparation XXXIII starting from 5-trifluoromethyl-1H-indole, the expected product was obtained as a light yellow solid (quantitative yield). M.p.=84-86° C.

### Example 80

### 5-[[1-[[3-(1,1-dimethylethyl)-phenyl]sulfonyl]-3-methyl-5-trifluoromethyl-1H-indol-2-yl]-hydroxymethyl]-thiophene-2-carboxylic acid methyl ester

Under an argon atmosphere, 240 mg (4.5 mM) of a solution of n-butyllithium (c=1.6 M in hexane) was added slowly to a solution of 1.12 g (3 mM) of 1-[[3-(1,1-dimethylethyl)phenyl]sulfonyl]-3-methyl-5-trifluoromethyl-1H-indole (preparation XXXIII) in 12 mL of tetrahydrofuran cooled to 0° C. The reaction mixture was stirred for 15 min at 0° C., then added dropwise at −78° C. to a solution of 433 mg (2.62 mM) of 5-formyl-thiophene-2-carboxylic acid methyl ester in 12 mL of tetrahydrofuran. The mixture was stirred for 30 minutes at −70° C., then diluted with a saturated aqueous solution of NH₄Cl and extracted three times with dichloromethane. The combined organic fractions was dried over magnesium sulfate and evaporated under reduced pressure. The residue was purified by silica gel chromatography, eluting with a cyclohexane/ethyl acetate mixture (95/5; v/v), then cyclohexane/ethyl acetate (90/10, v/v). The fractions containing the expected product were combined and concentrated to dryness under reduced pressure to give 1020 mg of 5-[[1-[[3-(1,1-dimethylethyl)phenyl]sulfonyl]-3-methyl-5-trifluoromethyl-1H-indol-2-yl]-hydroxymethyl]-thiophene-2-carboxylic acid methyl ester as an orange oil (yield=62%).

¹H NMR (300 MHz, DMSOd₆) δ=8.34 (d, 1H), 7.69 (m, 5H), 7.6 (d, 1H), 7.46 (t, 1H), 6.95 (m, 2H), 6.78 (d, 1H), 3.78 (s, 3H), 2.22 (s, 3H), 1.14 (s, 9H).

### Example 81

### 2-[[1-[[3-(1,1-dimethylethyl)-phenyl]sulfonyl]-3-methyl-5-trifluoromethyl-1H-indol-2-yl]-hydroxymethyl]-thiazole-4-carboxylic acid ethyl ester

Working similarly to example 80 starting from 2-formyl-thiazole-4-carboxylic acid methyl ester and the compound obtained in preparation XXXIII, the expected product was obtained as an orange oil (yield 40%).

¹H NMR (300 MHz, DMSOd₆) δ=8.53 (s, 1H), 8.25 (d, 1H), 8.03 (d, 1H), 7.91 (s, 2H), 7.70 (m, 2H), 7.48 (m, 2H), 6.98 (m, 1H), 4.28 (m, 2H), 2.04 (s, 3H), 1.31 (t, 3H), 1.25 (s, 9H).

### Example 82

### 4-{[1-[[3-(1,1-dimethylethyl)-phenyl]sulfonyl]-5-trifluoromethyl-1H-indol-2-yl]-hydroxymethyl}-1-methyl-1H-pyrrol-2-yl-carboxylic acid (1,1-dimethyl-ethyl)ester

Working similarly to example 80 starting from the compound obtained in preparation XXXIV and the compound obtained according to preparation XXXV, the expected product was obtained as a colourless oil (yield 9%).

¹H NMR (300 MHz, DMSOd₆) δ=8.23 (d, 1H), 8.02 (s, 1H), 7.64 (m, 4H), 7.46 (d, 1H), 6.92 (s, 1H), 6.88 (d, 1H), 6.63 (d, 1H), 6.29 (d, 1H), 5.95 (d, 1H), 3.74 (s, 3H), 1.47 (s, 9H), 1.15 (s, 9H).

### Example 83

### 5-[[1-[[3-(1,1-dimethylethyl)-phenyl]sulfonyl]-3-methyl-5-trifluoromethyl-1H-indol-2-yl]methyl]-thiophene-2-carboxylic acid methyl ester

1.05 g (9.02 mM) of triethylsilane, 0.02 g of trifluoroacetic acid and 1.28 g (9.02 mM) of boron diethyl ether trifluoride were added dropwise, successively, to a solution of 1.02 g (1.8 mM) of 5-[[1-[[3-(1,1-dimethylethyl)-phenyl]sulfonyl]-3-methyl-5-trifluoromethyl-1H-indol-2-yl]-hydroxymethyl]-thiophene-2-carboxylic acid methyl ester (example 80) in 10.2 mL of dichloromethane. The reaction was instantaneous and the reaction mixture was evaporated, then purified by silica gel chromatography, eluting with a cyclohexane/ethyl acetate mixture (95/5; v/v). The fractions containing the expected product were combined and concentrated to dryness under reduced pressure to give 490 mg of 5-[[1-[[3-(1,1-dimethylethyl)phenyl]sulfonyl]-3-methyl-5-trifluoromethyl-1H-indol-2-yl]methyl]-thiophene-2-carboxylic acid methyl ester as a yellow oil (yield=49%).

¹H NMR (300 MHz, DMSOd₆) δ=8.29 (d, 1H), 7.97 (s, 1H), 7.72 (d, 1H), 7.68 (d, 1H), 7.60 (d, 1H), 7.56 (m, 1H), 7.52 (m, 1H), 7.40 (t, 1H), 6.95 (d, 1H), 4.72 (s, 2H), 3.75 (s, 3H), 2.80 (s, 3H), 1.12 (s, 9H).

### Example 84

### 2-[[1-[[3-(1,1-dimethylethyl)-phenyl]-sulfonyl]-3-methyl-5-trifluoro-methyl-1H-indol-2-yl]methyl]-thiazole-4-carboxylic acid ethyl ester

204 mg (1.72 mM) of SOCl₂ was added to a solution of 200 mg (0.34 mM) of 2-[[1-[[3-(1,1-dimethylethyl)-phenyl]sulfonyl]-3-methyl-5-trifluoromethyl-1H-indol-2-yl]-hydroxymethyl]-thiazole-4-carboxylic acid ethyl ester (example 81) in 2 mL of dichloromethane and 12 mg of dimethylformamide (0.17 mM) cooled to 5° C., then the reaction mixture was stirred for 24 hours at room temperature. As reaction was incomplete, 204 mg (1.72 mM) of SOCl₂ was added twice at a 24-hour interval. The solution was then evaporated under reduced pressure. The crude product was suspended in 10 mL of hydrochloric acid and 109.15 mg (1.67 mM) of zinc was added. The reaction mixture was stirred for three days at room temperature. After extracting three times with ethyl acetate, the combined organic layers were dried over magnesium sulfate and evaporated under reduced pressure. The residue was purified by silica gel chromatography, eluting with a cyclohexane/ethyl acetate mixture (90/10; v/v) then (80/20) and the fractions containing the expected product were combined and concentrated to dryness under reduced pressure to give 58 mg of 2-[[1-[[3-(1,1-dimethylethyl)phenyl]sulfonyl]-3-methyl-5-trifluoromethyl-1H-indol-2-yl]methyl]-thiazole-4-carboxylic acid ethyl ester as a colourless solid (yield=31%).

¹H NMR (300 MHz, DMSOd₆) δ=8.35 (s, 1H), 8.30 (d, 1H), 8.00 (s, 1H), 7.64 (m, 3H), 7.58 (d, 1H), 7.43 (t, 1H), 4.88 (s, 2H), 4.28 (q, 2H), 2.29 (s, 3H), 1.29 (t, 3H), 1.15 (s, 9H).

### Example 85

### 5-[[1-[[3-(1,1-dimethylethyl)-phenyl]sulfonyl]-3-methyl-5-trifluoromethyl-1H-indol-2-yl]methyl]-thiophene-2-carboxylic acid

192 mg (8.01 mM) of lithium hydroxide was added to a solution of 490 mg (0.89 mM) of 5-[[1-[[3-(1,1-dimethylethyl)-phenyl]sulfonyl]-3-methyl-5-trifluoromethyl-1H-indol-2-yl]methyl]-thiophene-2-carboxylic acid methyl ester prepared in example 83 in 10 mL of tetrahydrofuran and 5 mL of water. The mixture was stirred for 4 days at room temperature and then acidified with a solution of 1N hydrochloric acid. After extracting twice with dichloromethane, the combined organic layers were dried over magnesium sulfate, and evaporated under reduced pressure. The residue was purified by silica gel chromatography, eluting with a cyclohexane/ethyl acetate mixture (80/20; v/v) then progressively up to cyclohexane/ethyl acetate (50/50; v/v). The fractions containing the expected product were combined and concentrated to dryness under reduced pressure to give 250 mg of 5-[[1-[[3-(1,1-dimethylethyl)-phenyl]sulfonyl]-3-methyl-5-trifluoromethyl-1H-indol-2-yl]methyl]-thiophene-2-carboxylic acid as a white solid (yield=52%). M.p.=171° C.

### Example 86

### 2-[[1-[[3-(1,1-dimethylethyl)-phenyl]sulfonyl]-3-methyl-5-(trifluoro-methyl)-1H-indol-2-yl]methyl]-thiazole-4-carboxylic acid

Working similarly to example 85 starting from the compound of example 84, the expected product was obtained as a white solid (yield 65%). M.p.=60° C.

### Example 87

### 4-[[1-[[3-(1,1-dimethylethyl)-phenyl]sulfonyl]-5-trifluoromethyl-1H-indol-2-yl]methyl]-1-methyl-pyrrol-2-yl-carboxylic acid

Working similarly to example 83 starting from the compound of example 82, the expected product was obtained as a beige solid (yield 58%). M.p.=160° C.

### Preparation XXXVI

### [1-[[3-(1,1-dimethylethyl)-phenyl]sulfonyl]-5-trifluoromethyl-1H-indol-2-yl]-acetonitrile

35.34 mg (0.11 mM) of tetrabutylammonium bromide and 107 mg (1.64 mM) of potassium cyanide were added to a solution of 520 mg (1.10 mM) of 2-bromomethyl-1-[[3-(1,1-dimethylethyl)-phenyl]sulfonyl]-5-trifluoromethyl-1H-indole obtained according to preparation XXIX in 4 mL of dichloromethane and 1 mL of water, and the reaction mixture was stirred overnight at room temperature. The reaction mixture was then hydrolysed with a saturated aqueous solution of Na₂CO₃ and extracted twice with dichloromethane. The combined organic layers were dried over magnesium sulfate, and evaporated under reduced pressure. As reaction was incomplete, the residue obtained was redissolved in 4 mL of dichloromethane and 1 mL of water, in the presence of 35.34 mg (0.11 mM) of tetrabutylammonium bromide and 107 mg (1.64 mM) of potassium cyanide for 4 hours at room temperature. After hydrolysis with a saturated aqueous solution of Na₂CO₃ and extraction twice with dichloromethane, the combined organic layers were dried over magnesium sulfate, and evaporated under reduced pressure to give 420 mg of [1-[[3-(1,1-dimethylethyl)-phenyl]sulfonyl]-5-trifluoromethyl-1H-indol-2-yl]-acetonitrile as a brown oil (yield 91%).

¹H NMR (300 MHz, DMSOd₆) δ=8.22 (d, 1H), 8.06 (s, 1H), 7.83 (m, 2H), 7.73 (m, 2H), 7.52 (t, 1H), 7.09 (s, 1H), 4.60 (s, 2H), 1.19 (s, 9H).

### Preparation XXXVII

### 2-[1-[[3-(1,1-dimethylethyl)-phenyl]sulfonyl]-5-trifluoromethyl-1H-indol-2-yl]-thioacetamide

0.75 mL (4 mM) of diethyl dithiophosphate was added to a solution of 420 mg (1 mM) of [1-[[3-(1,1-dimethylethyl)-phenyl]sulfonyl]-5-trifluoromethyl-1H-indol-2-yl]-acetonitrile (preparation XXXVI) in 4 mL of tetrahydrofuran and 8 mL of water. The reaction mixture was stirred overnight at 85° C. As reaction was incomplete, 744 mg (3.9 mM) of diethyl dithiophosphate was added, then the reaction mixture was kept under stirring for 7 hours at 85° C. The reaction mixture was hydrolysed with a saturated aqueous solution of Na₂CO₃ and extracted with ethyl acetate. The combined organic layers were dried over magnesium sulfate, and evaporated under reduced pressure. The residue was purified by silica gel chromatography, eluting with a cyclohexane/ethyl acetate mixture (90/10; v/v). The fractions containing the expected product were combined and concentrated to dryness under reduced pressure to give 320 mg of 2-[1-[[3-(1,1-dimethylethyl)-phenyl]sulfonyl]-5-trifluoromethyl-1H-indol-2-yl]-thioacetamide as an orange oil (yield=71%).

¹H NMR (300 MHz, DMSOd₆) δ=9.70 (s broad, 1H), 9.43 (s broad, 1H), 8.20 (d, 1H), 8.01 (s, 1H), 7.75 (m, 3H), 7.63 (d, 1H), 7.52 (t, 1H), 6.83 (s, 1H), 4.33 (s, 2H), 1.20 (s, 9H).

### Example 88

### 2-[[1-[[3-(1,1-dimethylethyl)-phenyl]sulfonyl]-5-trifluoromethyl-1H-indol-2-yl]methyl]-thiazole-4-carboxylic acid ethyl ester

21.45 mg (0.11 mM) of ethyl bromopyruvate was added to a solution of 50 mg (0.11 mM) of 2-[1-[[3-(1,1-dimethylethyl)-phenyl]sulfonyl]-5-trifluoromethyl-1H-indol-2-yl]-thioacetamide (preparation XXXVII) in 5 mL of ethanol. The reaction mixture was stirred overnight at room temperature, then the solvent was evaporated to give 57 mg of 2-[[1-[[3-(1,1-dimethylethyl)-phenyl]sulfonyl]-5-trifluoromethyl-1H-indol-2-yl]methyl]-thiazole-4-carboxylic acid ethyl ester as a yellow oil (yield=94%).

¹H NMR (300 MHz, DMSO) 8.42 (s, 1H), 8.28 (d, 1H), 8.03 (s, 1H), 7.72 (m, 4H), 7.48 (t, 1H), 6.91 (s, 1H), 4.91 (s, 2H), 4.29 (q, 2H), 1.30 (t, 3H), 1.18 (s, 9H).

### Example 89

### 2-[[1-[[3-(1,1-dimethylethyl)-phenyl]sulfonyl]-5-trifluoromethyl-1H-indol-2-yl]methyl]-thiazole-4-carboxylic acid

Working similarly to example 85 starting from the compound of example 88, the expected product was obtained as a brown oil (yield 33%).

¹H NMR (400 MHz, DMSOd₆) δ=12.95 (s broad, 1H), 8.35 (s, 1H), 8.27 (d, 1H), 8.03 (s, 1H), 7.72 (m, 4H), 7.48 (t, 1H), 6.91 (s, 1H), 4.89 (d, 2H), 1.18 (s, 9H).

### Preparation XXXVIII

### 5-(1-hydroxy-prop-2-ynyl)-thiophene-2-carboxylic acid methyl ester

40 mL of ethynylmagnesium bromide was added dropwise to a solution of 1.7 g (10 mM) of methyl ester of 5-formyl-thiophene-2-carboxylic acid in 17 mL of tetrahydrofuran cooled to 0° C., then the mixture was stirred for 30 minutes at 0° C. The solution was poured into 100 mL of a saturated aqueous solution of NH₄Cl and extracted three times with ethyl acetate. The combined organic layers were dried over magnesium sulfate, and evaporated under reduced pressure to give 2 g of 5-(1-hydroxy-prop-2-ynyl)-thiophene-2-carboxylic acid methyl ester as a brown solid (quantitative yield). M.p.=67° C.

### Example 90

### 5-[[1-[[3-(1,1-dimethylethyl)-phenyl]sulfonyl]-5-trifluoromethyl-1H-indol-2-yl]-hydroxymethyl]-thiophene-2-carboxylic acid methyl ester

Working similarly to preparation XXV starting from the compound obtained according to preparation XXXVIII and the compound obtained according to preparation XI, the expected product was obtained as an orange paste (yield 38%).

¹H NMR (300 MHz, DMSOd₆) δ=8.25 (d, 1H), 8.04 (s, 1H), 7.79 (m, 1H), 7.72 (m, 3H), 7.67 (d, 1H), 7.48 (t, 1H), 7.10 (d, 1H), 6.99 (d, 1H), 6.95 (s, 1H), 6.70 (d, 1H), 3.80 (s, 3H), 1.17 (s, 9H).

### Example 91

### 5-[[1-[[3-(1,1-dimethylethyl)-phenyl]sulfonyl]-5-trifluoromethyl-1H-indol-2-yl]hydroxymethyl]-thiophene-2-carboxylic acid

Working similarly to example 85 starting from the compound of example 90, the expected product was obtained as a brown solid (yield 66%). M.p.=90° C.

### Example 92

### 5-[[1-[[3-(1,1-dimethylethyl)-phenyl]sulfonyl]-5-trifluoromethyl-1H-indol-2-yl]-methyl]-thiophene-2-carboxylic acid

Working similarly to example 83 starting from the compound of example 91, the expected product was obtained as a white solid (yield 37%). M.p.=110° C.

### Example 93

### 5-[[1-[[3-(1,1-dimethylethyl)-phenyl]sulfonyl]-5-trifluoromethyl-1H-indol-2-yl]-carbonyl]-thiophene-2-carboxylic acid methyl ester

136.4 mg (0.36 mM) of pyridinium dichromate was added to a solution of 200.0 mg (0.36 mM) of 5-[[1-[[3-(1,1-dimethylethyl)-phenyl]sulfonyl]-5-trifluoromethyl-1H-indol-2-yl]-hydroxymethyl]-thiophene-2-carboxylic acid obtained in example 90 in 2.00 mL of dichloromethane, then the reaction mixture was stirred overnight at room temperature. The reaction mixture was filtered on a Whatman nylon membrane and the solid was rinsed with dichloromethane. The filtrate was concentrated under reduced pressure and the residue was purified by silica gel chromatography using cyclohexane/ethyl acetate (90/10; v/v) as eluent. The fractions containing the expected product were combined and concentrated to dryness under reduced pressure. 5-[[1-[[3-(1,1-dimethylethyl)-phenyl]sulfonyl]-5-trifluoro methyl-1H-indol-2-yl]-carbonyl]-thiophene-2-carboxylic acid methyl ester was obtained as an orange solid (186.00 mg; yield: 93%).

¹H NMR (300 MHz, DMSOd₆) δ=8.31 (d, 1H), 8.18 (s, 1H), 7.96 (m, 1H), 7.86 (m, 5H), 7.59 (m, 2H), 3.91 (s, 3H), 1.25 (s, 9H).

### Example 94

### 5-[[1-[[3-(1,1-dimethylethyl)-phenyl]sulfonyl]-5-trifluoromethyl-1H-indol-2-yl]-carbonyl]-thiophene-2-carboxylic acid

Working similarly to example 85 starting from the compound of example 93, the expected product was obtained as a yellow solid (yield 41%). M.p.=217° C.

### Preparation XXXIX

### 5-(1-hydroxy-1-methyl-prop-2-ynyl)-thiophene-2-carboxylic acid

Working similarly to preparation XXXVIII starting from 5-acetyl-thiophene-2-carboxylic acid, the expected product was obtained as a beige solid (yield 99%).

¹H NMR (300 MHz, DMSOd₆) δ=7.56 (d, 1H), 7.12 (d, 1H), 6.65 (s, 1H), 3.64 (s, 1H), 1.72 (s, 3H).

### Example 95

### 5-[1-[1-[[3-(1,1-dimethylethyl)-phenyl]sulfonyl]-5-(trifluoromethyl)-1H-indol-2-yl]-1-hydroxyethyl]-thiophene-2-carboxylic acid

Working similarly to preparation XXV starting from the compound obtained according to preparation XXXIX and the compound obtained according to preparation XI, the expected product was obtained as a yellow solid (yield 95%). M.p.=80° C.

### Example 96

### 5-[1-[1-[[3-(1,1-dimethylethyl)-phenyl]sulfonyl]-5-(trifluoromethyl)-1H-indol-2-yl]-1-ethenyl]-thiophene-2-carboxylic acid

Working similarly to example 83 starting from the compound of example 95, the expected product was obtained as a white solid (yield 62%). M.p.=195° C.

### Example 97

### 4-[[5-chloro-1-[[4-(1-methylethyl)phenyl]sulfonyl]-1H-indol-2-yl]methyl]-2-methoxybenzoic acid, methyl ester

Working similarly to example 1, starting from 4-(bromomethyl)-2-methoxybenzoic acid methyl ester and the compound obtained according to preparation V, the expected product was obtained as a yellow oil (yield=28%).

¹H NMR (DMSOd₆, 250 MHz) δ=1.13 (d, 6H), 2.91 (sept, 1H), 3.73 (s, 3H), 3.78 (s, 3H), 4.45 (s, 2H), 6.41 (s, 1H), 6.82 (dd, 1H), 7.02 (d, 1H), 7.33 (dd, 1H), 7.38 (d, 2H), 7.59 (dd, 1H), 7.62 (d, 1H), 7.70 (d, 2H), 8.06 (d, 1H).

### Example 98

### 4-[[5-chloro-1-[[4-(1-methylethyl)phenyl]sulfonyl]-1H-indol-2-yl]methyl]-2-methoxybenzoic acid

Working similarly to example 2, starting from the compound of example 97, 2-methoxy-4-(isopropyl-phenylsulfonyl]-5-(chloro)-1H-indol-2-yl]methyl]benzoic acid was obtained as a yellow solid (yield=99%). M.p.=67° C.

### Example 99

### 4-[[5-chloro-1-[[4-(1-methylethyl)phenyl]sulfonyl]-1H-indol-2-yl]methyl]-2-hydroxybenzoic acid

Working similarly to example 56, starting from the compound of example 98, the desired product was obtained as a grey solid (yield=90%). M.p.=139° C.

### Example 100

### 4-[1-[1[[4-(1-methylethyl)phenyl]sulfonyl]-5-(chloro)-1H-indol-2-yl]-1-hydroxyethyl]-benzoic acid, methyl ester

Working similarly to example 30 starting from the compound obtained in preparation XIV and preparation XV, the expected compound was obtained as a white foam (yield=69%). M.p.=163° C.

### Example 101

### 4-[1-[1[[4-(1-methylethyl)phenyl]sulfonyl]-5-(chloro)-1H-indol-2-yl]-1-ethenyl]-benzoic acid, methyl ester

Working similarly to example 31 starting from the compound of example 100, the expected product was obtained as a yellow paste (yield=47%).

¹H NMR (DMSOd₆, 250 MHz) δ=1.13 (d, 6H), 2.88 (sept, 1H), 3.85 (s, 3H), 5.86 (s, 1H), 6.12 (s, 1H), 6.93 (d, 1H), 7.36 (m, 4H), 7.43 (dd, 1H), 7.53 (d, 2H), 7.71 (d, 1H), 7.88 (d, 2H), 8.07 (d, 1H).

### Example 102

### 4-[1-[1[[4-(1-methylethyl)phenyl]sulfonyl]-5-(chloro)-1H-indol-2-yl]-1-ethenyl]-benzoic acid

Working similarly to example 2, starting from the compound of example 101, the desired acid was obtained as a beige powder (yield=34%). M.p.=236° C.

Working similarly to preparation X starting from the corresponding aniline, the compounds of preparations XL, XLI, XLII, XLIII, XLIV and XLV were obtained.

### Preparation XL

### N-[2-iodo-4-(tert-butyl)-phenyl]-3-(1,1-dimethylethyl)benzenesulfonamide

Appearance: brown oil. Yield: 93%

¹H NMR (DMSOd₆, 300 MHz) δ=1.20 (s, 9H), 1.22 (s, 9H), 6.97 (d, 1H), 7.36 (dd, 1H), 7.40 (t, 1H), 7.51 (t, 1H), 7.58 (dt, 1H), 7.69 (dd, 1H), 7.73 (d, 1H), 9.55 (s, 1H).

### Preparation XLI

### N-[2-iodo-4-bromo-phenyl]-3-(1,1-dimethylethyl)benzenesulfonamide

Appearance: black solid. Yield: quantitative. M.p.=145° C.

### Preparation XLII

### N-[2-iodo-4-(trifluoromethyl)-5-fluoro-phenyl]-3-(1,1-dimethylethyl)benzenesulfonamide

Appearance: yellow oil. Yield: 92%

¹H NMR (DMSOd₆, 300 MHz) δ=1.24 (s, 9H), 7.19 (d, 1H), 7.53 (t, 1H), 7.64 (dd, 2H), 7.72 (d, 1H), 8.11 (d, 1H).

### Preparation XLIII

### N-[2-iodo-4-methyl-phenyl]-3-(1,1-dimethylethyl)benzenesulfonamide

Appearance: yellow paste, Yield: quantitative

¹H NMR (DMSOd₆, 300 MHz) δ=1.24 (s, 9H), 2.21 (s, 3H), 6.90 (d, 1H), 7.11 (dd, 1H), 7.50 (m, 3H), 7.64 (dd, 1H), 7.68 (td, 1H), 9.55 (s, 1H).

### Preparation XLIV

### N-[2-iodo-3-chloro-4-chloro-phenyl]-3-(1,1-dimethylethyl)benzenesulfonamide

Appearance: beige solid. Yield: 65%. M.p.=148° C.

### Preparation XLV

### N-[2-iodo-6-fluoro-phenyl]-3-(1,1-dimethylethyl)benzenesulfonamide

Appearance: white solid. Yield: 69%. M.p.=133° C.

### Example 103

### 4-[(RS)-hydroxy[1-[[3-(1,1-dimethylethyl)phenyl]sulfonyl]-5-(tert-butyl)-1H-indol-2-yl]methyl]benzoic acid, methyl ester

Working similarly to example 30 starting from the compound obtained in preparation XL and 4-(1-hydroxy-2-propynyl)benzoic acid, the expected compound was obtained as a brown oil (yield=40%).

¹H NMR (DMSOd₆, 300 MHz) δ=1.15 (s, 9H), 1.26 (s, 9H), 3.85 (s, 3H), 6.34 (d, 1H), 6.44 (d, 1H), 6.56 (s, 1H), 7.39 (dd, 1H), 7.45 (t, 1H), 7.49 (m, 3H), 7.65 (m, 2H), 7.74 (t, 1H), 7.93 (m, 3H).

### Example 104

### 4-[(RS)-hydroxy[1-[[3-(1,1-dimethylethyl)phenyl]sulfonyl]-5-(bromo)-1H-indol-2-yl]methyl]benzoic acid, methyl ester

Working similarly to example 30 starting from the compound obtained in preparation XLI, the expected compound was obtained as a brown solid (yield=77%).

¹H NMR (DMSOd₆, 300 MHz) δ=1.19 (s, 9H), 3.85 (s, 3H), 6.48 (m, 2H), 6.60 (d, 1H), 7.46 (m, 2H), 7.50 (d, 2H), 7.66 (dt, 1H), 7.72 (dt, 1H), 7.79 (d, 1H), 7.83 (t, 1H), 7.94 (d, 2H), 7.98 (d, 1H).

### Example 105

### 4-[(RS)-hydroxy[1-[[3-(1,1-dimethylethyl)phenyl]sulfonyl]-5-(trifluoro)-6-fluoro-1H-indol-2-yl]methyl]benzoic acid, methyl ester

Working similarly to example 30 starting from the compound obtained in preparation XLII, the expected compound was obtained as a yellow oil (yield=80%).

¹H NMR (DMSOd₆, 300 MHz) δ=1.21 (s, 9H), 3.86 (s, 3H), 6.47 (d, 1H), 6.56 (d, 1H), 6.66 (s, 1H), 7.50 (m, 3H), 7.77 (td, 2H), 7.94 (d, 3H), 8.08 (m, 2H).

### Example 106

### 4-[(RS)-hydroxy[1-[[3-(1,1-dimethylethyl)phenyl]sulfonyl]-5-(methyl)-1H-indol-2-yl]methyl]benzoic acid, methyl ester

Working similarly to example 30 starting from the compound obtained in preparation XLIII, the expected compound was obtained as a yellow oil (yield=38%).

¹H NMR (DMSOd₆, 300 MHz) δ=1.17 (s, 9H), 2.31 (s, 3H), 3.85 (s, 3H), 6.37 (s broad, 1H), 6.45 (s broad, 1H), 6.52 (s, 1H), 7.13 (dd, 1H), 7.30 (s, 1H), 7.44 (t, 1H), 7.50 (d, 2H), 7.61 (td, 1H), 7.67 (td, 1H), 7.78 (t, 1H), 7.88 (d, 1H), 7.93 (d, 2H).

### Example 107

### 4-[(RS)-hydroxy[1-[[3-(1,1-dimethylethyl)phenyl]sulfonyl]-4-(chloro)-5-(chloro)-1H-indol-2-yl]methyl]benzoic acid, methyl ester

Working similarly to example 30 starting from the compound obtained in preparation XLIV, the expected compound was obtained as a yellow oil (yield=85%).

¹H NMR (DMSOd₆, 300 MHz) δ=1.20 (s, 9H), 3.86 (s, 3H), 6.48 (d, 1H), 6.57 (s, 1H), 6.62 (d, 1H), 7.52 (m, 4H), 7.66 (td, 1H), 7.74 (td, 1H), 7.84 (t, 1H), 7.94 (d, 2H), 8.04 (d, 1H).

### Example 108

### 4-[[1-[[3-(1,1-dimethylethyl)phenyl]sulfonyl]-5-(tert-butyl)-1H-indol-2-yl]methyl]benzoic acid, methyl ester

Working similarly to example 31 starting from the compound of example 103, the expected compound was obtained as a yellow oil (yield=73%).

¹H NMR (DMSOd₆, 400 MHz) δ=1.13 (s, 9H), 1.27 (s, 9H), 3.84 (s, 3H), 4.45 (s, 2H), 6.46 (s, 1H), 7.35 (d, 2H), 7.39 (dd, 1H), 7.44 (t, 1H), 7.47 (d, 1H), 7.57 (td, 1H), 7.58 (d, 1H), 7.67 (td, 1H), 7.89 (d, 2H), 7.96 (d, 1H).

### Example 109

### 4-[[1-[[3-(1,1-dimethylethyl)phenyl]sulfonyl]-5-(bromo)-1H-indol-2-yl]methyl]benzoic acid, methyl ester

Working similarly to example 31 starting from the compound of example 104, the expected compound was obtained as a colourless oil (yield=24%).

¹H NMR (DMSOd₆, 300 MHz) δ=1.18 (s, 9H), 3.85 (s, 3H), 4.49 (s, 2H), 6.43 (s, 1H), 7.36 (d, 2H), 7.47 (m, 2H), 7.59 (dd, 1H), 7.66 (t, 1H), 7.72 (dd, 1H), 7.74 (d, 1H), 7.91 (d, 2H), 8.00 (d, 1H).

### Example 110

### 4-[[1-[[3-(1,1-dimethylethyl)phenyl]sulfonyl]-5-(trifluoro)-6-fluoro-1H-indol-2-yl]methyl]benzoic acid, methyl ester

Working similarly to example 31 starting from the compound of example 105, the expected compound was obtained as a colourless oil (yield=48%).

¹H NMR (DMSOd₆, 400 MHz) δ=1.20 (s, 9H), 3.85 (s, 3H), 4.49 (s, 2H), 6.57 (s, 1H), 7.34 (d, 2H), 7.49 (t, 1H), 7.67 (d, 1H), 7.75 (m, 2H), 7.90 (d, 2H), 8.01 (d, 1H), 8.09 (d, 1H).

### Example 111

### 4-[[1-[[3-(1,1-dimethylethyl)phenyl]sulfonyl]-5-(methyl)-1H-indol-2-yl]methyl]benzoic acid, methyl ester

Working similarly to example 31 starting from the compound of example 106, the expected compound was obtained as a yellow oil (yield=70%).

¹H NMR (DMSOd₆, 300 MHz) δ=1.16 (s, 9H), 2.32 (s, 3H), 3.84 (s, 3H), 4.45 (s, 2H), 6.41 (s, 1H), 7.13 (dd, 1H), 7.27 (s, 1H), 7.36 (d, 2H), 7.43 (t, 1H), 7.54 (td, 1H), 7.63 (t, 1H), 7.68 (td, 1H), 7.90 (m, 3H).

### Example 112

### 4-[[1-[[3-(1,1-dimethylethyl)phenyl]sulfonyl]-4-(chloro)-5-(chloro)-1H-indol-2-yl]methyl]benzoic acid, methyl ester

Working similarly to example 31 starting from the compound of example 107, the expected compound was obtained as a colourless oil (yield=65%).

¹H NMR (DMSOd₆, 400 MHz) δ=1.19 (s, 9H), 3.85 (s, 3H), 4.53 (s, 2H), 6.57 (s, 1H), 7.37 (d, 2H), 7.47 (t, 1H), 7.55 (d, 1H), 7.59 (td, 1H), 7.69 (t, 1H), 7.74 (td, 1H), 7.90 (d, 2H), 8.06 (d, 1H).

### Example 113

### 4-[[1-[[3-(1,1-dimethylethyl)phenyl]sulfonyl]-7-fluoro-1H-indol-2-yl]methyl]benzoic acid, methyl ester

Working similarly to example 30 starting from the compound obtained in preparation XLV, 4-[(RS)-hydroxy[1-[[3-(1,1-dimethylethyl)phenyl]sulfonyl]-6-(fluoro)-1H-indol-2-yl]methyl]benzoic acid, methyl ester was obtained, and was used without further purification in the next reaction.

Working similarly to example 31, the expected compound was obtained as a white oil (yield=47%).

¹H NMR (DMSOd₆, 400 MHz) δ=1.17 (s, 9H), 3.85 (s, 3H), 4.55 (s, 2H), 6.61 (s broad, 1H), 7.06 (dd, 1H), 7.21 (m, 1H), 7.35 (d, 1H), 7.41 (d, 2H), 7.44 (t, 1H), 7.48 (d, 1H), 7.62 (s, 1H), 7.70 (dt, 1H), 7.92 (d, 2H).

### Example 114

### 4-[[1-[[3-(1-methylethyl)phenyl]sulfonyl]-5-(trifluoromethyl)-1H-indol-2-yl]methyl]benzoic acid

Working similarly to example 2, starting from the compound of example 108, the expected product was obtained as a white solid (yield=82%). M.p.=180° C.

### Example 115

### 4-[[1-[[3-(1-methylethyl)phenyl]sulfonyl]-5-(pyrrolidin)-1H-indol-2-yl]methyl]benzoic acid, methyl ester

204.22 mg (0.96 mM) of tribasic potassium phosphate, 14.36 mg (0.05 mM) of 2-(di-tert-butylphosphino)biphenyl, and 44.05 mg (0.05 mM) of Pd₂(dba)₃ were added to a solution of 260 mg (0.48 mM) of 4-[[1-[[3-(1,1-dimethylethyl)phenyl]sulfonyl]-5-(bromo)-1H-indol-2-yl]methyl]benzoic acid methyl ester of example 109 and 200 mL (2.41 mM) of pyrrolidine in 10 mL of toluene. The reaction mixture was heated for 1 hour at 100° C. in microwave equipment, then diluted in 50 mL of HCl (1N) and extracted twice with 100 mL of ethyl acetate. The combined organic layers were dried over magnesium sulfate and evaporated under reduced pressure. The residue was purified by silica gel chromatography, eluting with a cyclohexane/ethyl acetate mixture (95/5; v/v) then (80/20; v/v). The fractions containing the expected product were combined and concentrated to dryness under reduced pressure to give 40 mg of 4-[[1-[[3-(1-methyl ethyl)phenyl]sulfonyl]-5-(pyrrolidin)-1H-indol-2-yl]methyl]benzoic acid, methyl ester as a colourless paste (yield=15%).

¹H NMR (DMSOd₆, 300 MHz) δ=1.17 (s, 9H), 1.93 (m, 4H), 3.17 (m, 4H), 3.84 (s, 3H), 4.41 (s, 2H), 6.32 (s, 1H), 6.50 (d, 1H), 6.59 (dd, 1H), 7.36 (d, 2H), 7.42 (t, 1H), 7.50 (dd, 1H), 7.61 (t, 1H), 7.67 (d, 1H), 7.81 (d, 1H), 7.90 (d, 2H).

### Example 116

### 4-[[1-[[3-(1-methylethyl)phenyl]sulfonyl]-5-(pyrrolidin)-1H-indol-2-yl]methyl]benzoic acid

Working similarly to example 2, starting from the compound of example 115, the expected product was obtained as a beige powder (yield=34%). M.p.=90° C.

### Example 117

### 4-[[1-[[3-(1,1-dimethylethyl)phenyl]sulfonyl]-5-(trifluoro)-6-fluoro-1H-indol-2-yl]methyl]benzoic acid

Working similarly to example 2, starting from the compound of example 110, the expected product was obtained as a white powder (yield=34%). M.p.=175° C.

### Example 118

### 4-[[1-[[3-(1,1-dimethylethyl)phenyl]sulfonyl]-5-(methyl)-1H-indol-2-yl]methyl]benzoic acid

Working similarly to example 2, starting from the compound of example 111, the expected product was obtained as a beige powder (yield=24%). M.p.=161° C.

### Example 119

### 4-[[1-[[3-(1,1-dimethylethyl)phenyl]sulfonyl]-4-(chloro)-5-(chloro)-1H-indol-2-yl]methyl]benzoic acid

Working similarly to example 2, starting from the compound of example 112, the expected product was obtained as a white powder (yield=61%). M.p.=216° C.

Working similarly to example 12, starting from the corresponding sulfonyl chloride derivative and according to preparation VII, the compounds in the following examples were obtained.

### Example 120

### 4-[[1-[(6-methoxy-3-pyridinyl)sulfonyl]-5-(trifluoromethyl)-1H-indol-2-yl]methyl]benzoic acid

Appearance: beige paste. Yield=13%

¹H NMR (DMSOd₆, 250 MHz) δ=3.88 (s, 3H), 4.54 (s, 2H), 6.62 (s, 1H), 6.87 (d, 1H), 7.32 (d, 2H), 7.65 (dd, 1H), 7.86 (d, 2H), 7.98 (s, 1H), 7.99 (dd, 1H), 8.27 (d, 1H), 8.66 (d, 1H).

### Example 121

### 4-[[1-[4-chloro-3-methyl-phenylsulfonyl]-5-(trifluoromethyl)-1H-indol-2-yl]methyl]benzoic acid

Appearance: beige paste. Yield: 22%

¹H NMR (DMSOd₆, 250 MHz) δ=2.25 (s, 3H), 4.53 (s, 2H), 6.67 (s, 1H), 7.31 (d, 2H), 7.55 (d, 1H), 7.63 (m, 2H), 7.72 (d, 1H), 7.87 (d, 2H), 7.98 (d, 1H), 8.24 (d, 1H).

### Example 122

### 4-[[1-[benzofuran-2-sulfonyl]-5-(trifluoromethyl)-1H-indol-2-yl]methyl]benzoic acid

Appearance: beige paste. Yield: 10%

¹H NMR (DMSOd₆, 250 MHz) δ=4.56 (s, 2H), 6.69 (s, 1H), 7.35 (d, 2H), 7.39 (dd, 1H), 7.54 (t, 1H), 7.60 (d, 1H), 7.72 (t, 2H), 7.86 (d, 2H), 7.94 (d, 1H), 8.01 (s. 1H), 8.23 (d, 1H).

### Example 123

### 4-[[1-[4-propoxy-phenylsulfonyl]-5-(trifluoromethyl)-1H-indol-2-yl]methyl]benzoic acid

Appearance: beige paste. Yield: 27%

¹H NMR (DMSOd₆, 250 MHz) δ=0.93 (t, 3H), 1.68 (sept, 2H), 3.96 (t, 2H), 4.51 (s, 2H), 6.55 (s, 1H), 7.00 (d, 2H), 7.33 (d, 2H), 7.63 (d, 1H), 7.74 (d, 2H), 7.89 (d, 2H), 7.95 (s, 1H), 8.24 (d, 1H), 12.76 (s broad, 1H).

### Example 124

### 4-[[1-[3-chloro-4-difluoromethoxy-phenylsulfonyl]-5-(trifluoromethyl)-1H-indol-2-yl]methyl]benzoic acid

Appearance: beige paste. Yield: 10%

¹H NMR (DMSOd₆, 250 MHz) δ=4.54 (s, 2H), 6.70 (s, 1H), 7.32 (d, 2H), 7.38 (t, 1H), 7.43 (d, 1H), 7.66 (d, 1H), 7.80 (d, 1H), 7.87 (d, 2H), 7.90 (dd, 1H), 8.00 (s, 1H), 8.26 (d, 1H), 12.82 (s broad, 1H).

### Example 125

### 4-[[1-[4-methyl-3-oxo-3,4-dihydro-2H-benzo[1,4]oxazine-6-sulfonyl]-5-(trifluoromethyl)-1H-indol-2-yl]methyl]benzoic acid

Appearance: beige paste. Yield: 23%

¹H NMR (DMSOd₆, 250 MHz) δ=3.19 (s, 3H), 4.53 (s, 2H), 4.73 (s, 2H), 6.62 (s, 1H), 7.05 (d, 1H), 7.31 (m, 3H), 7.40 (dd, 1H), 7.65 (dd, 1H), 7.85 (d, 2H), 7.97 (s, 1H), 8.31 (d, 1H).

### Example 126

### 4-[[1-[3-difluoromethylsulfanyl-phenylsulfonyl]-5-(trifluoromethyl)-1H-indol-2-yl]methyl]benzoic acid

Appearance: beige paste. Yield: 14%

¹H NMR (DMSOd₆, 250 MHz) δ=4.51 (s, 2H), 6.64 (s, 1H), 7.31 (d, 2H), 7.63 (m, 2H), 7.87 (m, 5H), 7.65 (dd, 1H), 7.98 (s, 1H), 8.24 (d, 1H).

### Example 127

### 4-[[1-[4-isobutoxy-phenylsulfonyl]-5-(trifluoromethyl)-1H-indol-2-yl]methyl]benzoic acid

Appearance: beige paste. Yield: 32%

¹H NMR (DMSOd₆, 250 MHz) δ=0.93 (d, 6H), 1.97 (m, 1H), 3.77 (d, 2H), 4.51 (s, 2H), 6.55 (s, 1H), 7.00 (d, 2H), 7.33 (d, 2H), 7.63 (d, 1H), 7.75 (d, 2H), 7.88 (d, 2H), 7.95 (s, 1H), 8.24 (d, 1H), 12.87 (s broad, 1H).

### Example 128

### 4-[[1-[4-(3-methyl-butyl)-phenylsulfonyl]-5-(trifluoromethyl)-1H-indol-2-yl]methyl]benzoic acid

Appearance: beige paste

Yield: 33%

¹H NMR (DMSOd₆, 250 MHz)

δ=0.86 (d, 6H), 1.41 (m, 3H), 2.59 (m, 2H), 4.51 (s, 2H), 6.57 (s, 1H), 7.32 (d, 2H), 7.34 (d, 2H), 7.63 (d, 1H), 7.71 (d, 2H), 7.87 (d, 2H), 7.96 (s, 1H), 8.24 (d, 1H), 12.75 (s broad, 1H).

### Example 129

### 4-[[1-[4-(morpholine-4-carbonyl)benzenesulfonyl]-5-(trifluoromethyl)-1H-indol-2-yl]methyl]benzoic acid

Appearance: beige paste. Yield: 9%

¹H NMR (DMSOd₆, 250 MHz) δ=3.41 (m, 8H), 4.53 (s, 2H), 6.63 (s, 1H), 7.35 (d, 2H), 7.54 (d, 2H), 7.65 (dd, 1H), 7.87 (m, 4H), 7.98 (s, 1H), 8.26 (d, 1H), 12.87 (s broad, 1H).

### Example 130

### 4-[[1-[(6-phenoxy-3-pyridinyl)sulfonyl]-5-(trifluoromethyl)-1H-indol-2-yl]methyl]benzoic acid

Appearance: beige paste. Yield: 16%

¹H NMR (DMSOd₆, 500 MHz) δ=4.53 (s, 2H), 6.61 (s, 1H), 7.08 (d, 1H), 7.16 (d, 2H), 7.27 (t, 1H), 7.34 (d, 2H), 7.43 (td, 2H), 7.63 (dd, 1H), 7.88 (d, 2H), 7.99 (s, 1H), 8.19 (dd, 1H), 8.26 (d, 1H), 8.67 (d, 1H), 12.93 (s broad, 1H).

### Example 131

### 4-[[1-[4-(3,5-dimethyl-pyrazol-1-yl)-phenylsulfonyl]-5-(trifluoromethyl)-1H-indol-2-yl]methyl]benzoic acid

Appearance: beige paste. Yield: 21%

¹H NMR (DMSOd₆, 250 MHz) δ=2.16 (s, 3H), 2.34 (s, 3H), 4.53 (s, 2H), 6.13 (s, 1H), 6.58 (s, 1H), 7.36 (d, 2H), 7.66 (dd, 1H), 7.70 (d, 2H), 7.91 (m, 4H), 7.98 (s, 1H), 8.27 (d, 1H).

### Example 132

### 4-[[1-[(3,4-dihydro-2,2-dimethyl-2H-1-benzopyran-6-yl)sulfonyl]-5-(trifluoromethyl)-1H-indol-2-yl]methyl]benzoic acid

Appearance: beige paste. Yield: 19%

¹H NMR (DMSOd₆, 250 MHz) δ=1.24 (s, 6H), 1.71 (t, 2H), 2.64 (t, 2H), 4.52 (s, 2H), 6.61 (s, 1H), 6.76 (d, 1H), 7.29 (d, 2H), 7.45 (s, 1H), 7.49 (dd, 1H), 7.62 (dd, 1H), 7.86 (d, 2H), 7.97 (s, 1H), 8.24 (d, 1H), 12.63 (s broad, 1H).

### Example 133

### 4-[[1-[4-ethyl-phenylsulfonyl]-5-(trifluoromethyl)-1H-indol-2-yl]methyl]benzoic acid

Appearance: beige paste. Yield: 21%

¹H NMR (DMSOd₆, 250 MHz) δ=1.12 (t, 3H), 2.62 (q, 2H), 4.51 (s, 2H), 6.56 (s, 1H), 7.32 (d, 2H), 7.35 (d, 2H), 7.63 (dd, 1H), 7.73 (d, 2H), 7.86 (d, 2H), 7.95 (s, 1H), 8.24 (d, 1H), 12.65 (s broad, 1H).

### Example 134

### 4-[[1-[4-methylphenyl)sulfonyl]-5-(trifluoromethyl)-1H-indol-2-yl]methyl]benzoic acid

Appearance: beige paste. Yield: 20%

¹H NMR (DMSOd₆, 250 MHz) δ=2.32 (s, 3H), 4.51 (s, 2H), 6.55 (s, 1H), 7.33 (dd, 4H), 7.62 (dd, 1H), 7.72 (d, 2H), 7.88 (d, 2H), 7.94 (s, 1H), 8.23 (d, 1H), 12.60 (s broad, 1H).

### Example 135

### 4-[[1-[[6-(4-morpholinyl)-3-pyridinyl]sulfonyl]-5-(trifluoromethyl)-1H-indol-2-yl]methyl]benzoic acid

Appearance: beige paste. Yield: 20%

¹H NMR (DMSOd₆, 250 MHz) δ=3.60 (m, 8H), 4.53 (s, 2H), 6.57 (s, 1H), 6.75 (d, 1H), 7.32 (d, 2H), 7.63 (dd, 1H), 7.74 (dd, 1H), 7.88 (d, 2H), 7.96 (s, 1H), 8.25 (d, 1H), 8.47 (d, 1H), 12.82 (s broad, 1H).

### Example 136

### 4-[[1-[4-chloro-phenylsulfonyl]-5-(trifluoromethyl)-1H-indol-2-yl]methyl]benzoic acid

Appearance: beige paste. Yield: 22%

¹H NMR (DMSOd₆, 250 MHz) δ=4.50 (s, 2H), 6.60 (s, 1H), 7.33 (d, 2H), 7.59 (d, 2H), 7.64 (dd, 1H), 7.83 (d, 2H), 7.87 (d, 2H), 7.98 (s, 1H), 8.23 (d, 1H), 12.86 (s broad, 1H).

### Example 137

### 4-[[1-[4-fluoro-phenylsulfonyl]-5-(trifluoromethyl)-1H-indol-2-yl]methyl]benzoic acid

Appearance: beige paste. Yield: 8%

¹H NMR (DMSOd₆, 250 MHz) δ=4.51 (s, 2H), 6.58 (s, 1H), 7.35 (dd, 4H), 7.64 (dd, 1H), 7.90 (m, 4H), 7.97 (s, 1H), 8.24 (d, 1H), 12.91 (s broad, 1H).

### Example 138

### 4-[[1-[4-methoxy-phenylsulfonyl]-5-(trifluoromethyl)-1H-indol-2-yl]methyl]benzoic acid

Appearance: beige paste, Yield: 16%

¹H NMR (DMSOd₆, 250 MHz) δ=3.79 (s, 3H), 4.51 (s, 2H), 6.53 (s, 1H), 7.02 (d, 2H), 7.33 (d, 2H), 7.63 (dd, 1H), 7.77 (d, 2H), 7.88 (d, 2H), 7.94 (s, 1H), 8.25 (d, 1H), 12.94 (s broad, 1H).

### Example 139

### 4-[[1-[4-propyl-phenylsulfonyl]-5-(trifluoromethyl)-1H-indol-2-yl]methyl]benzoic acid

Appearance: beige paste. Yield: 37%

¹H NMR (DMSOd₆, 500 MHz) δ=0.83 (t, 3H), 1.52 (m, 2H), 2.56 (t, 2H), 4.51 (s, 2H), 6.56 (s, 1H), 7.31 (d, 2H), 7.34 (d, 2H), 7.63 (dd, 1H), 7.72 (d, 2H), 7.88 (d, 2H), 7.96 (s, 1H), 8.24 (d, 1H).

### Example 140

### 4-[[1-[4-pentyl-benzenesulfonyl]-5-(trifluoromethyl)-1H-indol-2-yl]methyl]benzoic acid

Appearance: beige paste. Yield: 23%

¹H NMR (DMSOd₆, 500 MHz) δ=0.82 (t, 3H), 1.19 (m, 2H), 1.25 (m, 2H), 1.50 (m, 2H), 2.58 (t, 2H), 4.51 (s, 2H), 6.56 (s, 1H), 7.32 (d, 2H), 7.34 (d, 2H), 7.63 (dd, 1H), 7.72 (d, 2H), 7.88 (d, 2H), 7.96 (s, 1H), 8.24 (d, 1H).

### Example 141

### 4-[[1-[(3-methylphenyl)sulfonyl]-5-(trifluoromethyl)-1H-indol-2-yl]methyl]benzoic acid

Appearance: beige paste. Yield: 31%

¹H NMR (DMSOd₆, 500 MHz) δ=2.26 (s, 3H), 4.54 (s, 2H), 6.63 (s, 1H), 7.34 (d, 2H), 7.42 (t, 1H), 7.50 (dd, 2H), 7.63 (d, 2H), 7.88 (d, 2H), 7.97 (s, 1H), 8.23 (d, 1H).

### Example 142

### 4-[[1-[4-trifluoromethoxy-phenylsulfonyl]-5-(trifluoromethyl)-1H-indol-2-yl]methyl]benzoic acid

Appearance: beige paste. Yield: 21%

¹H NMR (DMSOd₆, 500 MHz) δ=4.51 (s, 2H), 6.62 (s, 1H), 7.31 (d, 2H), 7.49 (d, 2H), 7.66 (dd, 1H), 7.86 (d, 2H), 7.96 (d, 2H), 7.99 (s, 1H), 8.25 (d, 1H).

### Example 143

### 4-[[1-[3-chloro-phenylsulfonyl]-5-(trifluoromethyl)-1H-indol-2-yl]methyl]benzoic acid

Appearance: beige paste. Yield: 20%

¹H NMR (DMSOd₆, 500 MHz) δ=4.54 (s, 2H), 6.71 (s, 1H), 7.33 (d, 2H), 7.56 (t, 1H), 7.63 (t, 1H), 7.66 (dd, 1H), 7.75 (dd, 1H), 7.79 (dd, 1H), 7.89 (d, 2H), 8.00 (s, 1H), 8.24 (d, 1H).

### Example 144

### 4-[[1-[4-phenoxy-phenylsulfonyl]-5-(trifluoromethyl)-1H-indol-2-yl]methyl]benzoic acid

Appearance: beige paste. Yield: 14%

¹H NMR (DMSOd₆, 500 MHz) δ=4.50 (s, 2H), 6.55 (s, 1H), 6.97 (d, 2H), 7.12 (d, 2H), 7.28 (t, 1H), 7.32 (d, 2H), 7.45 (d, 1H), 7.47 (d, 1H), 7.63 (dd, 1H), 7.83 (d, 2H), 7.88 (d, 2H), 7.97 (s, 1H), 8.24 (d, 1H).

### Example 145

### 4-[[1-[3-trifluoromethoxy-phenylsulfonyl]-5-(trifluoromethyl)-1H-indol-2-yl]methyl]benzoic acid

Appearance: beige paste. Yield: 22%

¹H NMR (DMSOd₆, 500 MHz) δ=4.53 (s, 2H), 6.67 (s, 1H), 7.33 (d, 2H), 7.68 (m, 4H), 7.87 (d, 3H), 7.99 (s, 1H), 8.25 (d, 1H).

### Example 146

### 4-[[1-[4′-chloro-biphenyl-3-sulfonyl]-5-(trifluoromethyl)-1H-indol-2-yl]methyl]benzoic acid

Appearance: beige paste. Yield: 10%

¹H NMR (DMSOd₆, 250 MHz) δ=4.56 (s, 2H), 6.65 (s, 1H), 7.33 (d, 2H), 7.53 (d, 2H), 7.62 (d, 2H), 7.65 (dd, 2H), 7.78 (dd, 1H), 7.85 (d, 2H), 7.98 (m, 3H), 8.28 (d, 1H).

### Example 147

### 4-[[1-[4-chloro-3-difluoromethoxy-phenylsulfonyl]-5-(trifluoromethyl)-1H-indol-2-yl]methyl]benzoic acid

Appearance: beige paste. Yield: 9%

¹H NMR (DMSOd₆, 250 MHz) δ=4.50 (s, 2H), 6.63 (s, 1H), 7.32 (d, 2H), 7.38 (t, 1H), 7.65 (m, 2H), 7.79 (d, 2H), 7.87 (d, 2H), 7.99 (s, 1H), 8.25 (d, 1H), 12.88 (s broad, 1H).

### Preparation XLVI

### 4-[[3-fluoro-5-trifluoromethyl)-1H-indol-2-yl]methyl]benzoic acid, methyl ester

1.28 g (3.60 mM) of 1-chloromethyl-4-fluoro-1,4-diazoniabicyclo[2.2.2]octane bis(tetrafluoroborate) was added, at 0° C., to a solution of 1 g (3 mM) of ester obtained according to preparation VII in 50 mL of acetonitrile. The reaction mixture was stirred at room temperature for 20 hours, then diluted in water and extracted with ethyl acetate. The organic layer was washed with a solution of HCl (1N) then NaCl. The combined organic layers were dried over magnesium sulfate and evaporated under reduced pressure. The residue was purified by silica gel chromatography, eluting with a cyclohexane/ethyl acetate mixture (85/15; v/v). The fractions containing the expected product were combined and concentrated to dryness under reduced pressure to give 310 mg of the desired product as an orange powder (yield=29%).

¹H NMR (DMSOd₆, 300 MHz) δ=3.83 (s, 3H), 4.23 (s, 2H), 7.40 (m, 3H), 7.49 (d, 1H), 7.85 (s, 1H), 7.93 (d, 2H), 11.45 (s broad, 1H).

### Example 148

### 4-[[1-[[3-(1,1-dimethylethyl)phenyl]sulfonyl]-3-fluoro-5-(trifluoromethyl)-1H-indol-2-yl]methyl]-benzoic acid, methyl ester

Working similarly to preparation I, starting from the compound obtained in preparation XLVI and 3-tert-butyl phenylsulfonyl chloride, the expected product was obtained in the form of a yellow oil (yield=72%).

¹H NMR (DMSOd₆, 400 MHz) δ=1.18 (s, 9H), 3.84 (s, 3H), 4.54 (s, 2H), 7.32 (d, 2H), 7.42 (t, 1H), 7.52 (d, 1H), 7.59 (t, 1H), 7.70 (d, 1H), 7.82 (d, 1H), 7.87 (d, 2H), 8.03 (s, 1H), 8.38 (d, 1H).

### Example 149

### 4-[[1-[[3-(1,1-dimethylethyl)phenyl]sulfonyl]-3-fluoro-5-(trifluoromethyl)-1H-indol-2-yl]methyl]-benzoic acid

Working similarly to example 2, starting from the compound of example 148, the expected product was obtained as yellow crystals (yield=36%). M.p.=158° C.

Working according to the procedure of example 12, starting from preparation VIII and from the corresponding sulfonylated derivative, the following examples were prepared.

### Example 150

### 4-[[1-[3-chloro-4-fluoro-phenylsulfonyl]-5-(chloro)-1H-indol-2-yl]methyl]benzoic acid

Appearance: beige paste. Yield: 7%

¹H NMR (DMSOd₆, 250 MHz) δ=4.49 (s, 2H), 6.55 (s, 1H), 7.31 (d, 2H), 7.35 (dd, 1H), 7.55 (t, 1H), 7.65 (d, 1H), 7.81 (d, 2H), 7.87 (d, 2H), 8.05 (d, 1H), 12.65 (s broad, 1H).

### Example 151

### 4-[[1-[biphenyl-4-sulfonyl]-5-(chloro)-1H-indol-2-yl]methyl]benzoic acid

Appearance: beige paste. Yield: 7%

¹H NMR (DMSOd₆, 250 MHz) δ=4.51 (s, 2H), 6.44 (s, 1H), 7.35 (d, 3H), 7.47 (m, 3H), 7.63 (dd, 2H), 7.67 (dd, 1H), 7.81 (m, 2H), 7.82 (d, 2H), 7.89 (d, 2H), 8.05 (d, 1H), 12.84 (s broad, 1H).

### Example 152

### 4-[[1-[4-propyl-phenylsulfonyl]-5-(chloro)-1H-indol-2-yl]methyl]benzoic acid

Appearance: beige paste. Yield: 26%

¹H NMR (DMSOd₆, 250 MHz) δ=0.83 (t, 3H), 1.52 (m, 2H), 2.57 (t, 2H), 4.47 (s, 2H), 6.40 (s, 1H), 7.33 (dd, 5H), 7.60 (dd, 1H), 7.68 (d, 2H), 7.87 (d, 2H), 8.03 (d, 1H), 12.85 (s broad, 1H).

### Example 153

### 4-[[1-[3-fluoro-4-fluoro-phenylsulfonyl]-5-(chloro)-1H-indol-2-yl]methyl]benzoic acid

Appearance: beige paste. Yield: 10%

¹H NMR (DMSOd₆, 250 MHz) δ=4.49 (s, 2H), 6.49 (s, 1H), 7.34 (m, 3H), 7.59 (dd, 1H), 7.63 (d, 1H), 7.70 (m, 1H), 7.82 (dd, 1H), 7.87 (d, 2H), 8.05 (d, 1H), 12.89 (s broad, 1H).

### Example 154

### 4-[[1-[3-fluoro-phenylsulfonyl]-5-(chloro)-1H-indol-2-yl]methyl]benzoic acid

Appearance: beige paste. Yield: 18%

¹H NMR (DMSOd₆, 250 MHz) δ=4.49 (s, 2H), 6.49 (s, 1H), 7.34 (m, 3H), 7.58 (m, 5H), 7.88 (d, 2H), 8.03 (d, 1H), 12.80 (s broad, 1H).

### Example 155

### 4-[[1-[4-tert-butyl-phenylsulfonyl]-5-(chloro)-1H-indol-2-yl]methyl]benzoic acid

Appearance: beige paste. Yield: 21%

¹H NMR (DMSOd₆, 250 MHz) δ=1.22 (s, 9H), 4.47 (s, 2H), 6.43 (s, 1H), 7.29 (d, 2H), 7.34 (dd, 1H), 7.51 (d, 2H), 7.64 (m, 3H), 7.85 (d, 2H), 8.06 (d, 1H), 12.89 (s broad, 1H).

### Example 156

### 4-[[1-[4-trifluoromethoxy-phenylsulfonyl]-5-(chloro)-1H-indol-2-yl]methyl]benzoic acid

Appearance: beige paste. Yield: 10%

¹H NMR (DMSOd₆, 250 MHz) δ=4.47 (s, 2H), 6.46 (s, 1H), 7.32 (d, 2H), 7.35 (dd, 1H), 7.49 (d, 2H), 7.63 (d, 1H), 7.86 (d, 2H), 7.91 (d, 2H), 8.04 (d, 1H), 12.58 (s broad, 1H).

### Example 157

### 4-[[1-[2,3-dihydro-benzo[1,4]dioxin-6-sulfonyl]-5-(chloro)-1H-indol-2-yl]methyl]benzoic acid

Appearance: beige paste. Yield: 23%

¹H NMR (DMSOd₆, 250 MHz) δ=4.25 (m, 4H), 4.45 (s, 2H), 6.44 (s, 1H), 6.95 (d, 1H), 7.08 (dd, 1H), 7.30 (m, 4H), 7.61 (d, 1H), 7.87 (d, 2H), 8.03 (d, 1H), 12.88 (s broad, 1H).

### Example 158

### 4-[[1-[4-trifluoro-phenylsulfonyl]-5-(chloro)-1H-indol-2-yl]methyl]benzoic acid

Appearance: beige paste. Yield: 11%

¹H NMR (DMSOd₆, 250 MHz) δ=4.48 (s, 2H), 6.50 (s, 1H), 7.31 (d, 2H), 7.36 (dd, 1H), 7.64 (d, 1H), 7.84 (d, 2H), 7.87 (d, 2H), 7.96 (d, 2H), 8.05 (d, 1H), 12.87 (s broad, 1H).

### Example 159

### 4-[[1-[4-ethyl-phenylsulfonyl]-5-(chloro)-1H-indol-2-yl]methyl]benzoic acid

Appearance: beige paste. Yield: 24%

¹H NMR (DMSOd₆, 250 MHz) δ=1.12 (t, 3H), 2.62 (q, 2H), 4.48 (s, 2H), 6.40 (s, 1H), 7.34 (m, 5H), 7.60 (dd, 1H), 7.69 (d, 2H), 7.87 (d, 2H), 8.03 (d, 1H), 12.89 (s broad, 1H).

### Example 160

### 4-[[1-[4-chloro-phenylsulfonyl]-5-(chloro)-1H-indol-2-yl]methyl]benzoic acid

Appearance: beige paste. Yield: 19%

¹H NMR (DMSOd₆, 250 MHz) δ=4.46 (s, 2H), 6.44 (s, 1H), 7.34 (m, 3H), 7.59 (m, 3H), 7.80 (d, 2H), 7.88 (d, 2H), 8.02 (d, 1H), 12.89 (s broad, 1H).

### Example 161

### 4-[[5-chloro-1-[(3-methylphenyl)sulfonyl]-1H-indol-2-yl]methyl]benzoic acid

Appearance: beige paste. Yield: 24%

¹H NMR (DMSOd₆, 250 MHz) δ=2.26 (s, 3H), 4.49 (s, 2H), 6.46 (s, 1H), 7.34 (m, 3H), 7.42 (d, 1H), 7.48 (m, 2H), 7.58 (m, 1H), 7.61 (d, 1H), 7.88 (d, 2H), 8.01 (d, 1H).

### Example 162

### 4-[[1-[4-isopropoxy-phenylsulfonyl]-5-(chloro)-1H-indol-2-yl]methyl]benzoic acid

Appearance: beige paste. Yield: 6%

¹H NMR (DMSOd₆, 250 MHz) δ=1.22 (s, 3H), 1.25 (s, 3H), 4.46 (s, 2H), 4.67 (m, 1H), 6.39 (s, 1H), 6.97 (d, 2H), 7.33 (m, 3H), 7.60 (d, 1H), 7.68 (d, 2H), 7.88 (d, 2H), 8.03 (d, 1H), 12.89 (s broad, 1H).

### Example 163

### 4-[[5-chloro-1-(2-naphthalenylsulfonyl)-1H-indol-2-yl]methyl]benzoic acid

Appearance: beige paste. Yield: 9%

¹H NMR (DMSOd₆, 250 MHz) δ=4.56 (s, 2H), 6.43 (s, 1H), 7.32 (dd, 1H), 7.35 (d, 2H), 7.58 (d, 1H), 7.69 (m, 3H), 7.85 (d, 2H), 8.01 (t, 2H), 8.13 (t, 2H), 8.61 (d, 1H), 12.86 (s broad, 1H).

### Example 164

### 4-[[1-[3-chloro-phenylsulfonyl]-5-(chloro)-1H-indol-2-yl]methyl]benzoic acid

Appearance: beige paste. Yield: 16%

¹H NMR (DMSOd₆, 250 MHz) δ=4.49 (s, 2H), 6.54 (s, 1H), 7.33 (d, 2H), 7.35 (dd, 1H), 7.55 (t, 1H), 7.60 (t, 1H), 7.64 (d, 1H), 7.75 (m, 2H), 7.87 (d, 2H), 8.03 (d, 1H), 12.87 (s broad, 1H).

### Example 165

### 4-[[1-[4-methoxy-phenylsulfonyl]-5-(chloro)-1H-indol-2-yl]methyl]benzoic acid

Appearance: beige paste. Yield: 25%

¹H NMR (DMSOd₆, 250 MHz) δ=3.79 (s, 3H), 4.47 (s, 2H), 6.37 (s, 1H), 7.01 (d, 2H), 7.32 (dd, 1H), 7.34 (d, 2H), 7.58 (d, 1H), 7.74 (d, 2H), 7.89 (d, 2H), 8.03 (d, 1H), 12.89 (s broad, 1H).

### Example 166

### 4-[[1-[3-methoxy-phenylsulfonyl]-5-(chloro)-1H-indol-2-yl]methyl]benzoic acid

Appearance: beige paste. Yield: 27%

¹H NMR (DMSOd₆, 250 MHz) δ=3.74 (s, 3H), 4.47 (s, 2H), 6.46 (s, 1H), 7.14 (t, 1H), 7.24 (ddd, 1H), 7.33 (m, 4H), 7.45 (t, 1H), 7.61 (d, 1H), 7.87 (d, 2H), 8.03 (d, 1H), 12.89 (s broad, 1H).

### Example 167

### 4-[[1-[4-fluoro-phenylsulfonyl]-5-(chloro)-1H-indol-2-yl]methyl]benzoic acid

Appearance: beige paste. Yield: 19%

¹H NMR (DMSOd₆, 250 MHz) δ=4.47 (s, 2H), 6.42 (s, 1H), 7.87 (m, 5H), 7.61 (d, 1H), 7.90 (m, 4H), 8.03 (d, 1H), 12.89 (s broad, 1H).

### Example 168

### 4-[[5-chloro-1-[[4-(1,1-dimethylpropyl)phenyl]sulfonyl]-1H-indol-2-yl]methyl]benzoic acid

Appearance: beige paste. Yield: 19%

¹H NMR (DMSOd₆, 250 MHz) δ=0.53 (t, 3H), 1.18 (s, 6H), 1.56 (q, 2H), 4.46 (s, 2H), 6.43 (s, 1H), 7.29 (d, 2H), 7.34 (dd, 1H), 7.45 (d, 2H), 7.61 (d, 1H), 7.66 (d, 2H), 7.85 (d, 2H), 8.05 (d, 1H), 12.87 (s broad, 1H).

### Example 169

### 4-[[5-chloro-1-[(6-methoxy-3-pyridinyl)sulfonyl]-1H-indol-2-yl]methyl]benzoic acid

Appearance: beige paste. Yield: 19%

¹H NMR (DMSOd₆, 500 MHz) δ=3.88 (s, 3H), 4.50 (s, 2H), 6.46 (s, 1H), 6.88 (d, 1H), 7.33 (d, 2H), 7.34 (dd, 1H), 7.62 (d, 1H), 7.88 (d, 2H), 7.97 (dd, 1H), 8.06 (d, 1H), 8.63 (d, 12.90 (s broad, 1H).

### Example 170

### 4-[[1-[4-pentyl-phenylsulfonyl]-5-(chloro)-1H-indol-2-yl]methyl]benzoic acid

Appearance: beige paste. Yield: 10%

¹H NMR (DMSOd₆, 500 MHz) δ=0.82 (t, 3H), 1.23 (m, 4H), 1.51 (m, 2H), 2.58 (t, 2H), 4.46 (s, 2H), 6.39 (s, 1H), 7.33 (m, 5H), 7.59 (dd, 1H), 7.68 (d, 2H), 7.87 (d, 2H), 8.03 (d, 1H), 12.89 (s broad, 1H).

### Example 171

### 4-[[5-chloro-1-[(4-methylphenyl)sulfonyl]-1H-indol-2-yl]methyl]benzoic acid

Appearance: beige paste. Yield: 10%

¹H NMR (DMSOd₆, 250 MHz) δ=2.32 (s, 3H), 4.47 (s, 2H), 6.39 (s, 1H), 7.33 (m, 5H), 7.58 (d, 1H), 7.68 (d, 2H), 7.88 (d, 2H), 8.02 (d, 1H), 12.91 (s broad, 1H).

### Example 172

### 4-[[1-[3′-fluoro-biphenyl-4-sulfonyl]-5-(chloro)-1H-indol-2-yl]methyl]benzoic acid

Appearance: beige paste. Yield: 23%

¹H NMR (DMSOd₆, 500 MHz) δ=4.51 (s, 2H), 6.45 (s, 1H), 7.28 (m, 1H), 7.36 (m, 3H), 7.54 (m, 3H), 7.62 (dd, 1H), 7.86 (m, 6H), 8.09 (d, 1H), 12.90 (s broad, 1H).

### Example 173

### 4-[[5-chloro-1-[(3,4-dihydro-2,2-dimethyl-2H-1-benzopyran-7-yl)sulfonyl]-1H-indol-2-yl]methyl]-benzoic acid

Appearance: beige paste. Yield: 17%

¹H NMR (DMSOd₆, 500 MHz) δ=1.24 (s, 6H), 1.71 (t, 2H), 2.64 (t, 2H), 4.48 (s, 2H), 6.45 (s, 1H), 6.76 (d, 1H), 7.31 (d, 2H), 7.32 (dd, 1H), 7.43 (m, 1H), 7.46 (dd, 1H), 7.61 (d, 1H), 7.87 (d, 2H), 8.04 (d, 1H), 12.90 (s broad, 1H).

### Example 174

### 4-[[1-(1.3-benzodioxol-5-ylsulfonyl)-5-chloro-1H-indol-2-yl]methyl]benzoic acid

Appearance: beige paste. Yield: 30%

¹H NMR (DMSOd₆, 500 MHz) δ=4.48 (s, 2H), 6.13 (s, 2H), 6.42 (s, 1H), 7.00 (d, 1H), 7.19 (d, 1H), 7.32 (dd, 1H), 7.35 (d, 2H), 7.43 (dd, 1H), 7.60 (d, 1H), 7.89 (d, 2H), 8.03 (d, 1H), 12.88 (s broad, 1H).

### Example 175

### 4-[[5-chloro-1-[(6-phenoxy-3-pyridinyl)sulfonyl]-1H-indol-2-yl]methyl]benzoic acid

Appearance: beige paste. Yield: 6%

¹H NMR (DMSOd₆, 500 MHz) δ=4.49 (s, 2H), 6.45 (s, 1H), 7.08 (dd, 1H), 7.16 (d, 2H), 7.27 (t, 1H), 7.33 (dd, 1H), 7.35 (d, 2H), 7.44 (td, 2H), 7.63 (d, 1H), 7.88 (d, 2H), 8.05 (d, 1H), 8.15 (dd, 1H), 8.64 (d, 1H), 12.90 (s broad, 1H).

### Example 176

### 4-[[5-chloro-1-(ethylsulfonyl)-1H-indol-2-yl]methyl]benzoic acid

Appearance: beige paste. Yield: 24%

¹H NMR (DMSOd₆, 500 MHz) δ=1.04 (t, 3H), 3.47 (q, 2H), 4.38 (s, 2H), 6.34 (s, 2H), 7.32 (dd, 1H), 7.41 (d, 2H), 7.66 (d, 1H), 7.87 (d, 1H), 7.92 (d, 2H), 12.92 (s broad, 1H).

### Example 177

### 4-[[1-[benzofuran-2-sulfonyl]-5-(chloro)-1H-indol-2-yl]methyl]benzoic acid

Appearance: beige paste. Yield: 6%

¹H NMR (DMSOd₆, 250 MHz) δ=4.52 (s, 2H), 6.51 (s, 1H), 7.35 (d, 2H), 7.39 (dd, 2H), 7.53 (m, 1H), 7.61 (d, 1H), 7.65 (d, 1H), 7.74 (td, 1H), 7.87 (d, 2H), 7.90 (d, 1H), 8.02 (d, 1H), 12.86 (s broad, 1H).

### Example 178

### 4-[[5-chloro-1-[(3,4-dihydro-2H-1,5-benzodioxepin-7-yl)sulfonyl]-1H-indol-2-yl]methyl]methyl]benzoic acid

Appearance: beige paste. Yield: 25%

¹H NMR (DMSOd₆, 500 MHz) δ=2.10 (m, 2H), 4.14 (t, 2H), 4.21 (t, 2H), 4.46 (s, 2H), 6.48 (s, 1H), 7.01 (d, 1H), 7.08 (d, 1H), 7.31 (d, 2H), 7.34 (dd, 1H), 7.37 (dd, 1H), 7.63 (d, 1H), 7.88 (d, 2H), 8.02 (d, 1H), 12.88 (s broad, 1H).

### Example 179

### 4-[[1-[4′-fluoro-biphenyl-4-sulfonyl]-5-(chloro)-1H-indol-2-yl]methyl]benzoic acid

Appearance: beige paste. Yield: 9%

¹H NMR (DMSOd₆, 250 MHz) δ=4.50 (s, 2H), 6.44 (s, 1H), 7.32 (m, 5H), 7.61 (d, 1H), 7.80 (m, 8H), 8.09 (d, 1H), 12.91 (s broad, 1H).

### Preparation XLVII

### N-[2-iodo-4-chloro-phenyl]-methanesulfonamide

Working similarly to preparation IX, starting from 2-iodo-4-chloroaniline and methanesulfonyl chloride, the expected product was obtained in the form of a yellow oil (quantitative yield).

¹H NMR (DMSOd₆, 300 MHz) δ=3.06 (s, 3H), 7.38 (d, 1H), 7.48 (dd, 1H), 7.97 (d, 1H), 9.34 (s, 1H).

### Example 180

### 4-[[5-chloro-1-(methylsulfonyl)-1H-indol-2-yl]methyl]benzoic acid, methyl ester

Working similarly to example 48, starting from the compound obtained in preparation XLVII, the expected compound was obtained as a white solid (yield=48%). M.p.=143° C.

### Example 181

### 4-[[5-chloro-1-(methylsulfonyl)-1H-indol-2-yl]methyl]benzoic acid

Working similarly to example 2, starting from the ester of example 180, the expected compound was obtained as a white powder (yield=88%). M.p.=244° C.

### Preparation XLVIII

### 3,3-dimethyl-2,3-dihydro-benzofuran-5-sulfonic chloride

5.48 mL (102.56 mM) of sulfuric acid in solution in 48 mL of ethyl ether was added dropwise at 0° C. to a solution of 3.80 g (25.64 mM) of 3-dimethyl-2,3-dihydro-benzofuran in 8 mL of ethyl ether. The reaction mixture was stirred at room temperature for 30 minutes and then at reflux temperature for 20 hours and evaporated under vacuum.

The reaction mixture was then diluted in 250 mL of dichloromethane and treated with 15.27 mL (177.86 mM) of oxalyl chloride and 1.28 mL of dimethyl formamide. The reaction mixture was stirred at room temperature for 16 hours, then evaporated under reduced pressure and the residue obtained was purified by silica gel chromatography, eluting with cyclohexane and then with a cyclohexane/ethyl acetate mixture (95/5; v/v). The fractions containing the expected product were combined and concentrated to dryness under reduced pressure to give 720 mg of 3,3-dimethyl-2,3-dihydro-benzofuran-5-sulfonic chloride as a yellow oil (yield=11%).

¹H NMR (DMSOd₆, 300 MHz) δ=1.29 (s, 6H), 4.22 (s, 2H), 6.67 (dd, 1H), 7.37 (dd, 1H), 7.41 (dd, 1H).

### Preparation XLIX

### N-(2-iodo-4-trifluoromethyl-phenyl)-3,3-dimethyl-2,3-dihydro-benzofuran-5-sulfonamide

Working similarly to preparation X, starting from 4-trifluoromethyl-2-iodo-aniline and 3,3-dimethyl-2,3-dihydro-benzofuran-5-sulfonyl chloride (obtained in preparation XLVIII) the expected compound was obtained as a yellow oil (yield=63%).

¹H NMR (DMSOd₆, 300 MHz) δ=1.25 (s, 6H), 4.33 (s, 2H), 6.93 (d, 1H), 7.30 (d, 1H), 7.41 (d, 1H), 7.55 (dd, 1H), 7.71 (d, 1H), 8.10 (s, 1H), 9.74 (s, 1H).

### Example 182

### 4-[[[1-[3,3-dimethyl-2,3-dihydro-benzofuran-5-sulfonyl]-5-(chloro)-1H-indol-2-yl]methyl]benzoic acid, methyl ester

Working similarly to example 48, starting from the compound obtained in preparation XLIX, the expected compound was obtained as a white powder (yield=50%). M.p.=160° C.

### Example 183

### 4-[[[1-[3,3-dimethyl-2,3-dihydro-benzofuran-5-sulfonyl]-5-(chloro)-1H-indol-2-yl]methyl]-benzoic acid

Using the same conditions as in Example 2, starting of the ester from example 182, the expected compound was obtained as white crystals (yield=99%). M.p.=190° C.

### Example 184

### 3-[[1-[[3-(1,1-dimethylethyl)phenyl]sulfonyl]-3-methyl-5-(trifluoromethyl)-1H-indol-2-yl]hydroxymethyl]-benzoic acid, methyl ester

Working similarly to example 64, starting from the compound obtained in preparation XXXIII and 3-carbomethoxybenzaldehyde, the expected compound was obtained as a colourless paste (yield=37%).

¹H NMR (DMSOd₆, 300 MHz) δ=1.14 (s, 9H), 2.03 (s, 3H), 3.82 (s, 3H), 6.51 (d, 1H), 6.80 (d, 1H), 7.21-8.37 (m, 11H).

### Example 185

### 3-[[1-[[3-(1,1-dimethylethyl)phenyl]sulfonyl]-3-methyl-5-(trifluoromethyl)-1H-indol-2-yl]methyl]-benzoic acid, methyl ester

Working similarly to example 31, starting from example 184, the expected compound was obtained as a yellow paste (yield=25%).

¹H NMR (DMSOd₆, 300 MHz) δ=1.12 (s, 9H), 2.27 (s, 3H), 3.81 (s, 3H), 4.55 (s, 2H), 7.39 (m, 4H), 7.56 (t, 1H), 7.65 (td, 1H), 7.68 (s, 1H), 7.71 (dd, 1H), 7.78 (td, 1H), 7.98 (s, 1H), 8.31 (d, 1H).

### Example 186

### 3-[[1-[[3-(1,1-dimethylethyl)phenyl]sulfonyl]-3-methyl-5-(trifluoromethyl)-1H-indol-2-yl]methyl]-benzoic acid

Working similarly to example 2, starting from the ester of example 185, the expected compound was obtained as a white powder (yield=75%). M.p.=194° C.

### Example 187

### 4-[[1-[[3-(1,1-dimethylethyl)phenyl]sulfonyl]-3-methyl-5-(trifluoromethyl)-1H-indol-2-yl]hydroxymethyl]-benzoic acid, methyl ester

Working similarly to example 64 starting from the compound from the compound obtained in preparation XXXIII and methyl 4-formylbenzoate, the expected product was obtained as a yellow powder (yield 51%). M.p.=65° C.

### Example 188

### 4-[[1-[[3-(1,1-dimethylethyl)phenyl]sulfonyl]-3-methyl-5-(trifluoromethyl)-1H-indol-2-yl]methyl]-benzoic acid, methyl ester

Working similarly to example 31 starting from the compound of example 187, the expected product was obtained as a yellow resin (yield 87%).

¹H NMR (DMSOd₆, 300 MHz) δ=1.12 (s, 9H), 2.26 (s, 3H), 3.83 (s, 3H), 4.56 (s, 2H), 7.21 (d, 2H), 7.40 (m, 2H), 7.56 (s, 1H), 7.65 (td, 1H), 7.71 (dd, 1H), 7.83 (d, 2H), 7.97 (s, 1H), 8.30 (d, 1H).

### Example 189

### 4-[[1-[[3-(1,1-dimethylethyl)phenyl]sulfonyl]-3-methyl-5-(trifluoromethyl)-1H-indol-2-yl]methyl]-benzoic acid

Working similarly to example 2 starting from the ester of example 188, the expected product was obtained as a white powder (yield 91%). M.p.=90° C.

### Example 190

### 5-[[1-[[3-(1,1-dimethylethyl)-phenyl]sulfonyl]-5-trifluoromethyl-1H-indol-2-yl]-methyl]-thiophene-2-carboxylic acid, methyl ester

Working similarly to example 185 starting from the compound obtained in example 90, the expected product was obtained as a yellow oil (yield 93%).

¹H NMR (DMSOd₆, 400 MHz)

δ=1.16 (s, 9H), 3.78 (s, 3H), 4.73 (s, 2H), 6.83 (s, 1H), 7.05 (d, 1H), 7.47 (t, 1H), 7.66 (m, 4H), 7.71 (dd, 1H), 7.99 (s, 1H), 8.27 (d, 1H).

### Example 191

### 4-{[1-[[3-(1,1-dimethylethyl)-phenyl]sulfonyl]-5-trifluoromethyl-1H-indol-2-yl]-hydroxymethyl}-1-methyl-1H-pyrrol-2-yl-carboxylic acid, methyl ester

Working similarly to example 64 starting from the compound obtained in preparation III and 4-formyl-1-methyl-1H-pyrrole-2-carboxylate, the expected product was obtained as a yellow oil (yield 5%).

¹H NMR (DMSOd₆, 300 MHz) δ=1.15 (s, 9H), 3.69 (s, 3H), 3.78 (s, 3H), 6.00 (d, 1H), 6.31 (d, 1H), 6.66 (d, 1H), 6.94 (s, 1H), 6.99 (d, 1H), 7.44 (t, 1H). 7.59 (m, 2H), 7.67 (dd, 1H), 7.70 (s, 1H), 8.02 (s, 1H), 8.23 (d, 1H).

### Example 192

### 4-{[1-[[3-(1,1-dimethylethyl)-phenyl]sulfonyl]-5-trifluoromethyl-1H-indol-2-yl]-methyl}-1-methyl-1H-pyrrol-2-yl-carboxylic acid, methyl ester

Working similarly to example 31 starting from the ester of example 191, the expected product was obtained as a brown resin (yield 17%).

¹H NMR (DMSOd₆, 400 MHz) δ=1.16 (s, 9H), 3.70 (s, 3H), 3.79 (s, 3H), 4.19 (s, 2H), 6.61 (d, 1H), 6.67 (d, 1H), 6.97 (d, 1H), 7.48 (t, 1H), 7.61 (m, 3H), 7.72 (dd, 1H), 7.93 (s, 1H), 8.25 (d, 1H).

### Preparation L

### 1-[[3-(1,1-dimethylethyl)phenyl]sulfonyl]-1H-indole

Working similarly to preparation I starting from 1H-indole and 3-(1,1-dimethylethyl)-phenylsulfonyl chloride, the expected product was obtained as a brown oil (yield=99%).

¹H NMR (DMSOd₆, 300 MHz)

δ=1.22 (s, 9H), 6.84 (d, 1H), 7.25 (t, 1H), 7.35 (t, 1H), 7.51 (t, 1H), 7.60 (d, 1H), 7.72 (d, 1H), 7.78 (d, 1H), 7.84 (d, 1H), 7.87 (t, 1H), 7.97 (d, 1H).

### Example 193

### 4-[[1-[[3-(1,1-dimethylethyl)phenyl]sulfonyl]-1H-indol-2-yl]hydroxymethyl]-benzoic acid, methyl ester

Working similarly to example 64 starting from the compound obtained in preparation L and methyl 4-formylbenzoate, the expected product was obtained as a yellow oil (yield 34%).

¹H NMR (DMSOd₆, 300 MHz)

δ=1.17 (s, 9H), 3.85 (s, 3H), 6.41 (d, 1H), 6.48 (d, 1H), 6.57 (s, 1H), 7.22 (t, 1H), 7.32 (td, 1H), 7.45 (t, 1H), 7.52 (m, 3H), 7.67 (td, 2H), 7.81 (t, 1H), 7.94 (d, 2H), 8.02 (d, 1H).

### Example 194

### 4-[[1-[[3-(1,1-dimethylethyl)phenyl]sulfonyl]-1H-indol-2-yl]methyl]-benzoic acid, methyl ester

Working similarly to example 31 starting from the compound obtained in Example 193, the expected product was obtained as a yellow oil (yield 81%).

¹H NMR (DMSOd₆, 400 MHz) δ=1.15 (s, 9H), 3.85 (s, 3H), 4.47 (s, 2H), 6.47 (d, 1H), 7.23 (td, 1H), 7.32 (td, 1H), 7.37 (d, 2H), 7.44 (t, 1H), 7.49 (d, 1H), 7.57 (dt, 1H), 7.62 (t, 1H), 7.68 (dt, 1H), 7.90 (d, 2H), 8.05 (d, 1H).

### Example 195

### 4-[[1-[[3-(1,1-dimethylethyl)phenyl]sulfonyl]-1H-indol-2-yl]methyl]-benzoic acid

Working similarly to example 2 starting from the ester of example 194, the expected product was obtained as a white powder (yield 100%). M.p.=175° C.

### Preparation LI

(4-bromo-2-fluoro-5-methyl-phenyl)-1-[[3-(1,1-dimethylethyl)phenyl]sulfonyl]-5-(trifluoromethyl)-1H-indole-2-methanol

Working similarly to example 64 starting from the compound obtained in preparation III and 4-bromo-2-fluoro-5-methylbenzaldehyde, the expected compound was obtained as an orange foam (yield=65%).

¹H NMR (DMSOd₆, 300 MHz) δ=1.19 (s, 9H), 2.26 (s, 3H), 6.48 (d, 1H), 6.60 (d, 1H), 6.73 (s, 1H), 7.30 (d, 1H), 7.47 (t, 1H), 7.54 (d, 1H), 7.66 (td, 2H), 7.73 (dd, 1H), 7.88 (t, 1H), 8.02 (s, 1H), 8.27 (d, 1H).

### Preparation LII

### 2-[(4-bromo-2-fluoro-5-methyl-benzyl]-1-[[3-(1,1-dimethylethyl)phenyl]sulfonyl]-5-(trifluoromethyl)-1H-indole

Working similarly to example 31, starting from the compound obtained in preparation LI, the expected compound was obtained as a colourless oil (yield=50%).

¹H NMR (DMSOd₆, 300 MHz) δ=1.19 (s, 9H), 2.25 (s, 3H), 4.37 (s, 2H), 6.40 (s, 1H), 7.23 (d, 1H), 7.51 (t, 1H), 7.57 (d, 1H), 7.66 (d, 2H), 7.73 (t, 1H), 7.76 (d, 1H), 7.98 (s, 1H), 8.30 (d, 1H).

### Example 196

### 4-[[1-[[3-(1,1-dimethylethyl)phenyl]sulfonyl]-5-trifluoromethyl-1H-indol-2-yl]methyl]-5-fluoro-2-methyl-benzoic acid

Working similarly to example 67, starting from the compound obtained in preparation LII, the expected compound was obtained as a white solid (yield=44%). M.p.=195° C.

### Preparation LIII

### (4-bromo-2-methyl-phenyl)-1-[[3-(1,1-dimethylethyl)phenyl]sulfonyl]-5-(trifluoromethyl)-1H-indole-2-methanol

Working similarly to example 64 starting from the compound obtained in preparation III and 4-bromo-2-fluoro-5-methylbenzaldehyde, the expected compound was obtained as a white powder (yield=32%).

¹H NMR (DMSOd₆, 300 MHz) δ=1.20 (s, 9H), 2.27 (s, 3H), 6.30 (d, 1H), 6.50 (d, 1H), 6.64 (s, 1H), 7.13 (d, 1H), 7.34 (dd, 1H), 7.44 (d, 1H), 7.48 (t, 1H), 7.68 (td, 2H), 7.74 (dd, 1H), 7.94 (t, 1H), 8.02 (s, 1H), 8.27 (d, 1H).

### Preparation LIV

### 2-[(4-bromo-2-methyl-benzyl]-1-[[3-(1,1-dimethylethyl)phenyl]sulfonyl]-5-(trifluoromethyl)-1H-indole

Working similarly to example 31, starting from the compound obtained in preparation LIII, the expected compound was obtained as a colourless resin (yield=69%).

¹H NMR (DMSOd₆, 300 MHz) δ=1.19 (s, 9H), 2.05 (s, 3H), 4.28 (s, 2H), 6.14 (s, 1H), 7.05 (d, 1H), 7.35 (dd, 1H), 7.46 (d, 1H), 7.54 (t, 1H), 7.67 (dd, 1H), 7.73 (dd, 1H), 7.74 (d, 1H). 7.78 (td, 1H), 7.91 (s, 1H), 8.33 (d, 1H).

### Example 197

### 4-[[1-[[3-(1,1-dimethylethyl)phenyl]sulfonyl]-5-trifluoromethyl-1H-indol-2-yl]methyl]-3-methyl-benzoic acid

Working similarly to example 67, starting from the compound obtained in preparation LIV, the expected compound was obtained as a white powder (yield=31%). M.p.=135° C.

Working similarly to example 69, starting from the compound obtained in preparation XXIX and the appropriate boronic derivative, the compounds in the following examples were obtained.

### Example 198

### 4-[[1-[[3-(1,1-dimethylethyl)-phenyl]sulfonyl]-5-trifluoromethyl-1H-indol-2-yl]methyl]-benzenesulfonamide

Appearance: brown oil. Yield=10%

¹H NMR (DMSOd₆, 400 MHz) δ=1.18 (s, 9H), 4.52 (s, 2H), 6.60 (s, 1H), 7.33 (s, 2H), 7.41 (d, 2H), 7.48 (t, 1H), 7.60 (d, 1H), 7.66 (d, 1H), 7.72 (s, 2H), 7.77 (d, 2H), 7.96 (s, 1H), 8.27 (d, 1H).

### Example 199

### 3-[[1-[[3-(1,1-dimethylethyl)-phenyl]sulfonyl]-5-trifluoromethyl-1H-indol-2-yl]methyl]-4-fluorobenzoic acid

Appearance: beige paste. Yield=27%

¹H NMR (DMSOd₆, 300 MHz) δ=1.19 (s, 9H), 4.50 (s, 2H), 6.46 (s, 1H), 7.36 (t, 1H), 7.50 (t, 1H), 7.67 (m, 2H), 7.74 (m, 2H), 7.82 (dd, 1H), 7.94 (m, 2H), 8.30 (d, 1H), 13.01 (s broad, 1H).

### Example 200

### 3-[[1-[[3-(1,1-dimethylethyl)-phenyl]sulfonyl]-5-trifluoromethyl-1H-indol-2-yl]methyl]-5-fluorobenzoic acid

Appearance: beige paste. Yield=23%

¹H NMR (DMSOd₆, 300 MHz) δ=1.16 (s, 9H), 4.54 (s, 2H), 6.65 (s, 1H), 7.36 (dt, 1H), 7.47 (t, 1H), 7.54 (dd, 1H), 7.63 (m, 2H), 7.67 (m, 2H), 7.71 (dd, 1H), 7.97 (s, 1H), 8.58 (d, 1H), 13.18 (s broad, 1H).

### Example 201

### 3-[[1-[[3-(1,1-dimethylethyl)-phenyl]sulfonyl]-5-trifluoromethyl-1H-indol-2-yl]methyl]-5-fluorobenzoic acid

Appearance: beige paste. Yield=20%

¹H NMR (DMSOd₆, 300 MHz) δ=1.17 (s, 9H), 4.47 (s, 2H), 6.58 (s, 1H), 7.26 (dd, 1H), 7.49 (m, 2H), 7.67 (m, 5H), 7.95 (s, 1H), 8.27 (d, 1H), 13.21 (s broad, 1H).

### Example 202

### 3-[[1-[[3-(1,1-dimethylethyl)-phenyl]sulfonyl]-5-trifluoromethyl-1H-indol-2-yl]methyl]-6-methoxybenzoic acid

Appearance: beige paste. Yield=30%

¹H NMR (DMSOd₆, 300 MHz) δ=1.17 (s, 9H), 3.81 (s, 3H), 4.38 (s, 2H), 6.49 (s, 1H), 7.08 (d, 1H), 7.38 (dd, 1H), 7.48 (t, 1H), 7.51 (d, 1H), 7.64 (m, 2H), 7.69 (t, 1H), 7.73 (dd, 1H), 7.94 (s, 1H), 8.26 (d, 1H), 12.54 (s broad, 1H).

### Example 203

### 3-[[1-[[3-(1,1-dimethylethyl)-phenyl]sulfonyl]-5-trifluoromethyl-M-indol-2-yl]methyl]-4-chloro-6-fluorobenzoic acid

Appearance: beige paste. Yield=9%

¹H NMR (DMSOd₆, 300 MHz) δ=1.19 (s, 9H), 4.50 (s, 2H), 6.28 (s, 1H), 7.53 (t, 1H), 7.67 (m, 3H), 7.79 (m, 3H), 7.92 (s, 1H), 8.33 (d, 1H), 13.43 (s broad, 1H).

### Example 204

### 3-[[1-[[3-(1,1-dimethylethyl)-phenyl]sulfonyl]-5-trifluoromethyl-1H-indol-2-yl]methyl]-5-pyridine carboxylic acid

Appearance: brown oil. Yield=17%

¹H NMR (DMSOd₆, 400 MHz) δ=1.16 (s, 9H), 4.57 (s, 2H), 6.67 (s, 1H), 7.48 (t, 1H), 7.65 (t, 3H), 7.71 (dd, 1H), 7.96 (s, 1H), 8.06 (t, 1H), 8.27 (d, 1H), 8.73 (dd, 1H), 8.95 (dd, 1H), 13.47 (s broad, 1H).

### Example 205

### 4-[[1-[[3-(1,1-dimethylethyl)-phenyl]sulfonyl]-5-trifluoromethyl-1H-indol-2-yl]methyl]-3-chlorobenzoic acid

Appearance: beige paste. Yield=34%

¹H NMR (DMSOd₆, 300 MHz) δ=1.20 (s, 9H), 4.54 (s, 2H), 6.28 (s, 1H), 7.42 (d, 1H), 7.54 (t, 1H), 7.70 (dd, 2H), 7.78 (d, 2H), 7.86 (dd, 1H), 7.94 (d, 2H), 8.34 (d, 1H), 13.30 (s broad, 1H).

### Example 206

### 4-[[1-[[3-(1,1-dimethylethyl)-phenyl]sulfonyl]-5-trifluoromethyl-1H-indol-2-yl]methyl]-2-chlorobenzoic acid

Appearance: orange oil. Yield=4%

¹H NMR (DMSOd₆, 400 MHz) δ=1.17 (s, 9H), 4.49 (s, 2H), 6.64 (s, 1H), 7.25 (dd, 1H), 7.36 (d, 1H), 7.48 (t, 1H), 7.65 (m, 3H), 7.72 (d, 2H), 7.94 (s, 1H), 8.27 (d, 1H).

### Example 207

### 3-[[1-[[3-(1,1-dimethylethyl)-phenyl]sulfonyl]-5-trifluoromethyl-1H-indol-2-yl]methyl]-2-methoxybenzoic acid

Appearance: beige paste. Yield=30%

¹H NMR (DMSOd₆, 300 MHz) δ=1.20 (s, 9H), 3.53 (s, 3H), 4.39 (s, 2H), 6.22 (s, 1H), 7.16 (t, 1H), 7.35 (dd, 1H), 7.57 (t, 1H), 7.67 (m, 2H), 7.71 (dd, 1H), 7.79 (m, 2H), 7.92 (s, 1H), 8.33 (d, 1H), 12.95 (s broad, 1H).

### Example 208

### 3-[[1-[[3-(1,1-dimethylethyl)-phenyl]sulfonyl]-5-trifluoromethyl-1H-indol-2-yl]methyl]-4-methoxybenzoic acid

Appearance: beige paste. Yield=28%

¹H NMR (DMSOd₆, 300 MHz) δ=1.21 (s, 9H), 3.79 (s, 3H), 4.37 (s, 2H), 6.24 (s, 7.15 (d, 1H), 7.54 (t, 1H), 7.67 (m, 3H), 7.76 (m, 2H), 7.91 (m, 2H), 8.30 (d, 1H), 12.60 (s broad, 1H).

Working similarly to example 69, starting from the compound obtained in preparation XXX and the appropriate boronic derivative, the compounds in the following examples 209 and 210 were obtained.

### Example 209

### 4-[[1-[[4-(1-methylethyl)-phenyl]sulfonyl]-5-trifluoromethyl-1H-indol-2-yl]methyl]-2-chlorobenzoic acid

Appearance: beige paste. Yield=24%

¹H NMR (DMSOd₆, 300 MHz) δ=1.14 (d, 6H), 2.91 (sept, 1H), 4.51 (s, 2H), 6.65 (s, 1H), 7.26 (dd, 1H), 7.35 (d, 1H), 7.40 (d, 2H), 7.65 (dd, 1H), 7.72 (d, 2H), 7.74 (d, 1H), 7.98 (s, 1H), 8.27 (d, 1H), 13.31 (s broad, 1H).

### Example 210

### 3-[[1-[[3-(1,1-dimethylethyl)-phenyl]sulfonyl]-5-trifluoromethyl-1H-indol-2-yl]methyl]-6-fluorobenzoic acid

Appearance: beige paste. Yield=37%

¹H NMR (DMSOd₆, 300 MHz) δ=1.14 (d, 6H), 2.92 (sept, 1H), 4.48 (s, 2H), 6.58 (s, 1H), 7.25 (dd, 1H), 7.40 (d, 2H), 7.50 (m, 1H), 7.64 (dd, 1H), 7.69 (dd, 1H), 7.73 (d, 2H), 7.97 (s, 1H), 8.26 (d, 1H), 13.20 (s broad, 1H).

Working similarly to example 69, starting from the compound obtained in preparation XXXI and the appropriate boronic derivative, the compounds in the following examples 211 and 212 were obtained.

### Example 211

### 4-[[1-[[4-methyl-3,4-dihydro-2H-benzo[1,4]oxazin-6-yl]-sulfonyl]-5-trifluoromethyl-1H-indol-2-yl]methyl]-2-chlorobenzoic acid

Appearance: beige paste. Yield=37%

¹H NMR (DMSOd₆, 300 MHz) δ=2.75 (s, 3H), 3.23 (t, 2H), 4.23 (t, 2H), 4.50 (s, 2H), 6.64 (s, 1H), 6.73 (d, 1H), 6.86 (d, 1H), 7.00 (dd, 1H), 7.25 (dd, 1H), 7.34 (d, 1H), 7.64 (dd, 1H), 7.75 (d, 1H), 7.97 (s, 1H), 8.27 (d, 1H), 13.27 (s broad, 1H).

### Example 212

### 3-[[1-[[4-methyl-3,4-dihydro-2H-benzo[1,4]oxazin-6-yl]-sulfonyl]-5-trifluoromethyl-1H-indol-2-yl]methyl]-6-fluorobenzoic acid

Appearance: beige paste. Yield=22%

¹H NMR (DMSOd₆, 300 MHz) δ=2.75 (s, 3H), 3.24 (t, 2H), 4.23 (t, 2H), 4.47 (s, 2H), 6.57 (s, 1H), 6.73 (d, 1H), 6.86 (d, 1H), 7.01 (dd, 1H), 7.25 (dd, 1H), 7.49 (td, 1H), 7.63 (dd, 1H), 7.67 (dd, 1H), 7.96 (s, 1H), 8.27 (d, 1H), 13.20 (s broad, 1H).

Working similarly to example 69, starting from the compound obtained in preparation XXXII and the appropriate boronic derivative, the compounds in the following examples 213 and 214 were obtained.

### Example 213

### 3-[[1-[(2,3-dihydro-benzo[1,4]dioxin-6-yl)sulfonyl]-5-chloro-1H-indol-2-yl]methyl]-5-pyridine carboxylic acid

Appearance: beige paste. Yield=19%

¹H NMR (DMSOd₆, 300 MHz) δ=4.24 (m, 2H), 4.28 (m, 2H), 4.52 (s, 2H), 6.54 (s, 1H), 6.95 (d, 1H), 7.12 (d, 1H), 7.28 (dd, 1H), 7.35 (dd, 1H), 7.63 (d, 1H), 8.01 (t, 1H), 8.03 (d, 1H), 8.73 (d, 1H), 8.96 (d, 1H), 13.38 (s broad, 1H).

### Example 214

### 3-[[1-[(2,3-dihydro-benzo[1,4]dioxin-6-yl)sulfonyl]-5-trifluoromethyl-1H-indol-2-yl]methyl]-4-fluorobenzoic acid

Appearance: beige paste. Yield=39%

¹H NMR (DMSOd₆, 300 MHz) δ=4.23 (m, 2H), 4.28 (m, 2H), 4.42 (s, 2H), 6.47 (s, 1H), 6.94 (d, 1H), 7.06 (d, 1H), 7.27 (m, 2H), 7.34 (dd, 1H), 7.49 (m, 1H), 7.62 (d, 1H), 7.64 (dd, 1H), 8.03 (d, 1H), 13.20 (s broad, 1H).

### Example 215

### 4-[(RS)-hydroxy[1-[[3-(1,1-dimethylethyl)phenyl]sulfonyl]-5-(trifluoromethyl)-1H-indol-2-yl]methyl]benzoic acid, methyl ester

Working similarly to example 30 starting from the compound obtained in preparation XI and 4-(1-hydroxy-2-propynyl)benzoic acid, the desired product was obtained as an orange powder (yield=89%).

M.p.=60° C.

### Example 216

### 4-[[1-[[3-(1,1-dimethylethyl)phenyl]sulfonyl]-5-(trifluoromethyl)-1H-indol-2-yl]fluoromethyl]benzoic acid, methyl ester

A solution of 1 g (1.83 mM) of 4-[(RS)-hydroxy[1-[[3-(1,1-dimethylethyl)phenyl]sulfonyl]-5-(trifluoromethyl)-1H-indol-2-yl]methyl]benzoic acid, methyl ester (example 215) in solution in 16 mL of dichloromethane was added dropwise at −78° C. to a solution of 0.3 g (1.83 mM) of diethylaminosulfide trifluoride in 3 mL of dichloromethane cooled to −78° C. The reaction mixture was stirred at −78° C. for 30 minutes. Then the reaction mixture was diluted with 50 mL of dichloromethane. The organic layer was washed with 50 mL of Na₂CO₃ then twice with 50 mL of water. The combined organic layers were dried over magnesium sulfate and evaporated under reduced pressure. The residue was purified by silica gel chromatography, eluting with a cyclohexane/ethyl acetate mixture (90/10; v/v). The fractions containing the expected product were combined and concentrated to dryness under reduced pressure to give 858 mg of the desired ester as an orange powder (yield=85%).

¹H NMR (DMSOd₆, 300 MHz) δ=1.19 (s, 9H), 3.89 (s, 3H), 6.73 (d, 1H), 7.52 (t, 1H), 7.53 (d, 1H), 7.65 (d, 2H), 7.75 (d, 2H), 7.81 (td, 1H), 7.87 (t, 1H), 8.06 (d, 3H), 8.33 (d, 1H).

### Example 217

### 4-[[1-[[3-(1,1-dimethylethyl)phenyl]sulfonyl]-5-(trifluoromethyl)-1H-indol-2-yl]fluoromethyl]benzoic acid

Working similarly to example 2, starting from the compound of example 216, the expected product was obtained as an orange solid (yield=55%). M.p.=170° C.

### Example 218

### 4-[(RS)-hydroxy[1-[[3-(1,1-dimethylethyl)phenyl]sulfonyl]-5-(trifluoromethyl)-1H-indol-2-yl]methyl]benzoic acid

Working similarly to example 2, starting from the compound of example 215, the expected product was obtained as a beige powder (yield=94%). M.p.=110° C.

### Example 219

### 4-[1-[[3-(1,1-dimethylethyl)phenyl]sulfonyl]-5-(trifluoromethyl)-1H-indol-2-yl]carbonyl]benzoic acid

Working similarly to example 93, starting from the compound of example 218, the expected product was obtained as a white powder (yield: 8%). M.p.=180° C.

### Example 220

### 4-[1-[[3-(1,1-dimethylethyl)phenyl]sulfonyl]-5-(trifluoromethyl)-1H-indol-2-yl]methyl]benzonitrile

Working similarly to example 69, starting from the compound obtained in preparation XXIX and 4-cyanophenyl boronic acid, the expected product was obtained as a light yellow solid (yield=38%). M.p.=47° C.

### Example 221

### 4-[1-[3-(1,1-dimethylethyl)phenyl]sulfonyl]-5-(trifluoromethyl)-1H-indol-2H-tetrazol-5-yl-benzyl

819.73 mg (3.98 mM) of azidotrimethyltin was added to a solution of 565 mg (1.14 mM) of benzonitrile obtained in example 220 in 16.95 mL of ortho-xylene, then the reaction mixture was stirred overnight at reflux temperature. The reaction mixture was concentrated under reduced pressure and the residue was purified by silica gel chromatography using a gradient of cyclohexane/ethyl acetate (90/10; v/v) to (20/80; v/v) then using a gradient of dichloromethane/methanol (100/0; v/v) to (90/10; v/v). The fractions containing the expected product were combined and concentrated to dryness under reduced pressure to obtain the desired product as a white powder (yield=66%). M.p.=100° C.

### Example 222

### 3-[[4-[1-[[3-(1,1-dimethylethyl)phenyl]sulfonyl]-5-(trifluoromethyl)-1H-indol-2-yl]methyl]-phenyl]-4H-[1,2,4]oxadiazol-5-one

587.07 mg (3.58 mM) of hydroxylamine sulfate was added to a solution of 444 mg (0.89 mM) of the benzonitrile obtained in example 220 in 1 mL of ethanol and 1 mL of triethylamine, then the reaction mixture was heated overnight at 80° C. The reaction mixture was concentrated under reduced pressure and then dissolved in CH₂Cl₂. The salts were removed by filtration and the filtrate was evaporated.

343 μL (3.59 mM) of ethyl chloroformate was added at 0° C. to a solution of the residue thus formed, in 1.5 mL of pyridine, then the reaction mixture was stirred for thirty minutes at room temperature and overnight of reflux temperature. The reaction mixture was diluted with water and then extracted with ethyl acetate. The organic layer was washed with HCl (1N) then with NaCl. The combined organic layers were concentrated under reduced pressure and the residue was purified by silica gel chromatography using cyclohexane/ethyl acetate (90/10; v/v) to (20/80; v/v) as eluent. The fractions containing the expected product were combined and concentrated to dryness under reduced pressure to obtain the desired product as a white powder (yield: 12%). M.p.=175° C.

### Preparation LV

### 4-[1-[[3-(1,1-dimethylethyl)phenyl]sulfonyl]-5-(trifluoromethyl)-1H-indol-2-yl]methyl]benzaldehyde

Working similarly to example 69, starting from the compound obtained in preparation XXIX and 4-formylphenyl boronic acid, the expected product was obtained as a yellow oil (yield=22%).

¹H NMR (DMSOd₆, 300 MHz) δ=1.17 (s, 9H), 4.55 (s, 2H), 6.62 (s, 1H), 7.46 (m, 3H), 7.67 (m, 4H), 7.86 (d, 2H), 7.96 (s, 1H), 8.27 (d, 1H), 9.99 (s, 1H).

### Example 223

### 5-[1-[4-[1-[[3-(1,1-dimethylethyl)phenyl]sulfonyl]-5-(trifluoromethyl)-1H-indol-2-yl]methyl]-phenyl]-methylidene]-2-thioxo-thiazolin-4-one

A solution of 18.13 μL (0.18 mM) of piperidine and 10.52 μL (0.18 mM) of acetic acid in 5 mL of toluene was added to a solution of 131 mg (0.26 mM) of the benzaldehyde obtained in preparation LV and 34.93 mg (0.26 mM) of rhodanine in 1 mL of toluene, then the reaction mixture was stirred for 2 hours at 120° C. The reaction mixture was diluted with water and then extracted with ethyl acetate. The combined organic layers were concentrated under reduced pressure and the evaporation residue was purified by preparative liquid chromatography with detection by mass spectrometry (LC-MS), eluting with a H₂O/CH₃CN/0.1% TFA mixture. The fractions containing the expected product were combined and concentrated to dryness under reduced pressure to obtain the desired product as a yellow powder (yield=12%).

¹H NMR (DMSOd₆, 300 MHz) δ=1.17 (s, 9H), 4.50 (s, 2H), 6.60 (s, 1H), 7.39 (d, 2H), 7.47 (m, 2H), 7.56 (d, 2H), 7.60 (s, 1H), 7.63 (s, 1H), 7.66 (dd, 1H), 7.68 (t, 1H), 7.72 (td, 1H), 7.95 (s, 1H), 8.27 (d, 1H), 13.79 (s broad, 1H).

### Example 224

### N-[4-[[1-[[3-(1,1-dimethylethyl)phenyl]sulfonyl]-5-(trifluoromethyl)-1H-indol-2-yl]methyl]benzoyl]-hydrazinecarboxylic acid, tert-butyl ester

163.61 mg (0.85 mM) of EDCI and 116.17 mg (0.85 mM) of HOAT were added to a solution of 400 mg (0.78 mM) of 4-[[1-[[3-(1,1-dimethylethyl)phenyl]sulfonyl]-5-(trifluoromethyl)-1H-indol-2-yl]methyl]benzoic acid (example 49) in 2 mL of toluene, then the reaction mixture was stirred for 1 hour at room temperature. 0.12 mL (0.85 mM) of triethylamine and 112.79 mg (0.85 mM) of tert-butyl carbamate were then added and the mixture was stirred at room temperature for 16 hours. The reaction mixture was concentrated under reduced pressure and the evaporation residue was purified by preparative liquid chromatography with detection by mass spectrometry (LC-MS), eluting with a H₂O/CH₃CN/0.1% TFA mixture. The fractions containing the expected product were combined and concentrated to dryness under reduced pressure to obtain the desired product as a yellow oil (yield=75%).

¹H NMR (DMSOd₆, 300 MHz) δ=1.18 (s, 9H), 1.43 (s, 9H), 4.49 (s, 2H), 6.52 (s, 1H), 7.34 (d, 2H), 7.48 (t, 1H), 7.64 (m, 2H), 7.73 (d, 2H), 7.82 (d, 2H), 7.94 (s, 1H), 8.27 (d, 1H), 8.90 (s broad, 1H).

### Example 225

### 4-[[1-[[3-(1,1-dimethylethyl)phenyl]sulfonyl]-5-(trifluoromethyl)-1H-indol-2-yl]methyl]benzoic acid hydrazide

5 mL of trifluoroacetic acid was added to a solution of 360 mg (0.57 mM) of N-[4-[[1-[[3-(1,1-dimethylethyl)phenyl]sulfonyl]-5-(trifluoromethyl)-1H-indol-2-yl]methyl]benzoyl]-hydrazinecarboxylic acid tert-butyl ester obtained in example 224 in 5 mL of dichloromethane, then the reaction mixture was stirred for 2 hours at room temperature. The reaction mixture was concentrated under reduced pressure and the evaporation residue was purified by preparative liquid chromatography with detection by mass spectrometry (LC-MS), eluting with a H₂O/CH₃CN/0.1% TFA mixture. The fractions containing the expected product were combined and concentrated to dryness under reduced pressure to obtain the desired product as a colourless oil (yield=80%).

¹H NMR (DMSOd₆, 300 MHz) δ=1.18 (s, 9H), 4.52 (s, 2H), 6.60 (s, 1H), 7.38 (d, 2H), 7.49 (t, 1H), 7.69 (m, 4H), 7.83 (d, 2H), 7.95 (s, 1H), 8.27 (d, 1H), 10.93 (s broad, 1H).

### Example 226

### 5-[4-[[1-[[3-(1,1-dimethylethyl)phenyl]sulfonyl]-5-(trifluoromethyl)-1H-indol-2-yl]methyl]-phenyl]-3H-[1,3,4]oxadiazol-2-one

90 μL (0.61 mM) of triethylamine and 99.51 mg (0.61 mM) of 1,1′-carbonyldiimidazole were added at 0° C. to a solution of 250 mg (0.47 mM) of the acid hydrazide obtained in example 225 in 9.5 mL of dichloromethane, then the reaction mixture was stirred for 3 hours at room temperature. The reaction mixture was diluted with water and then extracted with dichloromethane. The organic layer was washed with HCl (1N) and then NaHCO₃. The combined organic layers were concentrated under reduced pressure and the residue was purified by preparative liquid chromatography with detection by mass spectrometry (LC-MS), eluting with a H₂O/CH₃CN/0.1% TFA mixture. The fractions containing the expected product were combined and concentrated to dryness under reduced pressure to obtain the desired product as a white solid (yield=28%). M.p.=92° C.

### Example 227

### N-[4-[[1-[[3-(1,1-dimethylethyl)phenyl]sulfonyl]-5-(trifluoromethyl)-1H-indol-2-yl]methyl]benzyl]-methanesulfonamide

74.37 mg (0.39 mM) of EDCI, 47.39 mg (0.39 mM) of 4-dimethylaminopyridine and 73.80 mg (0.78 mM) of methanesulfonamide were added to a solution of 200 mg (0.39 mM) of the acid obtained in example 49 in 910 mL of dichloromethane, then the reaction mixture was stirred for 20 hours at room temperature. The reaction mixture was concentrated under reduced pressure and the residue was purified by preparative liquid chromatography with detection by mass spectrometry (LC-MS), eluting with a H₂O/CH₃CN/0.1% TFA mixture. The fractions containing the expected product were combined and concentrated to dryness under reduced pressure to obtain the desired product as a white solid (yield=53%). M.p.=96° C.

### Example 228

### 3-[4-[[1-[[3-(1,1-dimethylethyl)phenyl]sulfonyl]-5-(trifluoromethyl)-1H-indol-2-yl]methyl]-phenyl]-4H-isoxazol-5-one

235.26 mg (1.45 mM) of 1,1′-carbonyldiimidazole was added to a solution of 680 mg (1.32 mM) of the acid obtained in example 49 in 5 mL of distilled tetrahydrofuran and then the mixture was stirred at room temperature for 4 hours.

75.46 mg (0.66 mM) of magnesium ethoxide was added to a solution of 174.26 mg (1.32 mM) of ethyl malonate in 5.0 ml of tetrahydrofuran, then the suspension was stirred for 4 hours at room temperature. The solvent was evaporated and the white solid obtained was then added by portions to the first mixture. Stirring was continued for 24 hours at room temperature and 100 mL of DCM was added and then the organic phase was washed three times with 50 mL of HCl/M. The organic layer was dried over MgSO₄ and the solvent was evaporated to obtain 700 mg of the desired product as an amorphous orange solid.

28.20 mg (0.85 mM) of hydroxylamine and then 0.85 ml (0.85 mM) of NaOH (1N) were added to a solution of 100 mg (0.17 mM) of the ester obtained above in 5.0 ml of methanol. The mixture was stirred for 3 days at room temperature. The mixture was diluted with 50 mL of ice and 5 mL of HCl and stirred for 30 minutes. It was filtered on a Whatman Autocup nylon membrane, washed with water and dried under vacuum. The solid was purified by silica gel chromatography using cyclohexane/ethyl acetate (80/20; v/v) to (50/50; v/v) as eluent. The fractions containing the expected product were combined and concentrated to dryness under reduced pressure to obtain the desired product as a white powder (yield=46%). M.p.=70° C.

### Example 229

### N-[4-[[1-[[3-(1,1-dimethylethyl)phenyl]sulfonyl]-5-(trifluoromethyl)-1H-indol-2-yl]methyl]benzoyl]-benzenesulfonamide

74.37 mg (0.39 mM) of EDCI, 47.39 mg (0.39 mM) of 4-dimethylaminopyridine and 121.96 mg (0.78 mM) of benzenesulfonamide were added to a solution of 200 mg (0.39 mM) of 4-[[1-[[3-(1,1-dimethylethyl)phenyl]sulfonyl]-5-(trifluoromethyl)-1H-indol-2-yl]methyl]-benzoic acid (example 49) in 10 mL of dichloromethane, then the reaction mixture was stirred overnight at room temperature. The reaction mixture was concentrated under reduced pressure and the residue was purified by preparative liquid chromatography with detection by mass spectrometry (LC-MS), eluting with H₂O/CH₃CN/0.1% TFA mixture. The fractions containing the expected product were combined and concentrated to dryness under reduced pressure to obtain the desired product as a white solid (yield=53%). M.p.=99° C.

### Preparation LVI

### 1-Bromo-3-(2-methoxymethoxy-1.1-dimethylethyl)-benzene

2.18 mL (24 mM) of bromomethoxymethane 5 mL was added dropwise to a solution of 5 g (21.8 mM) of 2-(3-bromophenyl)-2-methylpropan-1-ol in 50 mL of DCM and 5 mL of diisopropylamine cooled to 0° C. The reaction mixture was stirred at room temperature for 3 hours, then diluted in DCM and washed with water. The organic layer was dried over MgSO₄, concentrated under reduced pressure and the residue was purified by silica gel chromatography with a heptane/ethyl acetate mixture (100/0; v/v up to 65/35; v/v). The fractions containing the expected product were combined and concentrated to dryness under reduced pressure to give the desired product as a yellow oil (yield=41%). The resulting product was used without further purification in the following reaction.

### Preparation LVII

### 3-(2-methoxymethoxy-1,1-dimethylethyl)benzenesulfonyl chloride

3.28 mL (8.2 mM) of n-BuLi (solution 2.5 M in hexane) was added dropwise to a solution of 1.95 g (7.14 mM) de 1-Bromo-3-(2-methoxymethoxy-1,1-dimethylethyl)benzene, obtained in preparation LVI, in 10 mL of THF cooled to −65° C. The reaction mixture was stirred at −65° C. for 1 hour, further stirred at −30° C. for 1 hour and then added to a solution of 10 mL of sulfur dioxide condensed on THF and cooled to −78° C. The reaction mixture was gradually warmed up to room temperature and concentrated under vacuum. The residue was taken up and filtered. 0.66 mL (8.2 mM) of sulfuryle chloride was added dropwise to a suspension of the solid thus obtained in heptane at 0° C. The reaction mixture was stirred at 0° C. for 1 hour and the suspension was filtered off and concentrated to dryness under reduced pressure. The resulting sulfonyl chloride was used without further purification in the following reaction.

### Preparation LVIII

### 4-[[1-[3-(2-methoxymethoxy-1,1-dimethylethyl)phenyl]sulfonyl]-5-trifluoromethyl-1H-indol-2-yl]methyl}-benzoic acid methyl ester

Working similarly to example 12, starting from the sulfonyl chloride obtained in preparation LVII and of the compound obtained in preparation VII, the desired product was obtained as a colorless oil (yield=13%). The resulting product was used without further purification in the following reaction.

### Example 230

### 4-[[1-[3-(2-hydroxy-1,1-dimethylethyl)phenylsulfonyl]-5-trifluoromethyl-1H-indol-2-yl]methyl]]benzoic acid methyl ester

3 mL of TFA was added to a solution of 12 mg (0.02 mM) of 4-[[1-[3-(2-methoxymethoxy-1.1-dimethylethyl)phenyl]sulfonyl]-5-trifluoromethyl-1H-indol-2-yl]methyl}benzoic acid methyl ester obtained in preparation LVIII in 3 mL of DCM. The reaction mixture was stirred at room temperature for 18 hours, then concentrated under reduced pressure (quantitative yield). The resulting product was used without further purification in the following reaction.

### Example 231

### 4-[[1-[[3-(2-hydroxy-1,1-dimethylethyl)phenyl]sulfonyl]-5-(trifluoromethyl)-1H-indol-2-yl]methyl]benzoic acid

Working similarly to example 2, starting from the compound obtained in example 230, the desired product was obtained as a colorless paste (yield=14%).

¹H NMR (CDCl₃, 300 MHz) δ=1.20 (s, 6H), 3.47 (s, 2H), 4.48 (s, 2H), 6.40 (s, 1H), 7.31 (d, 2H), 7.40 (t, 1H), 7.55 (m, 2H), 7.68 (d, 1H), 7.95 (m, 2H), 7.94 (d, 2H), 8.34 (d, 1H).

The compounds according to the invention described above are presented in the following table:

TABLE 1

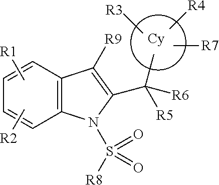

| Ex | R1 | R2 | R3 | R4 | R5 | R6 | R7 | R8 | R9 | Cy |
|---|---|---|---|---|---|---|---|---|---|---|
| 1 | 5-CF3 | H | 2-F | H | H | H | 4-COOMe | 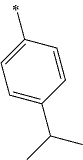 | H | 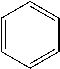 |
| 2 | 5-CF3 | H | 2-F | H | H | H | 4-COOH | 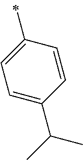 | H | 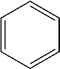 |
| 3 | 5-CF3 | H | 2- OMe | H | H | H | 4-COOMe | 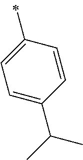 | H | 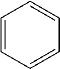 |
| 4 | 5-CF3 | H | 2- OMe | H | H | H | 4-COOH | 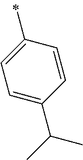 | H | 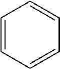 |
| 5 | 5-Cl | H | 2-F | H | H | H | 4-COOMe | 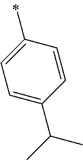 | H | 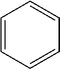 |
| 6 | 5-Cl | H | 2-F | H | H | H | 4-COOH | 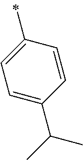 | H | 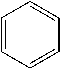 |
| 7 | 5-CF3 | H | H | H | H | H | 3-COOMe | 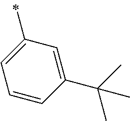 | H | 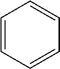 |
| 8 | 5-CF3 | H | H | H | H | H | 3-COOH | 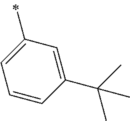 | H | 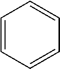 |
| 9 | 5-CF3 | H | 2-F | H | H | H | 4-COOMe | 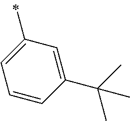 | H | 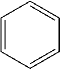 |
| 10 | 5-CF3 | H | 2-F | H | H | H | 4-COOH | 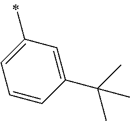 | H | 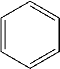 |
| 11 | 5-CF3 | H | 2- OMe | H | H | H | 4-COOMe | 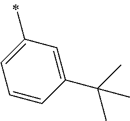 | H | 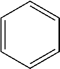 |
| 12 | 5-CF3 | H | H | H | H | H | 4-COOH | 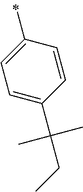 | H | 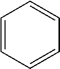 |
| 13 | 5-CF3 | H | H | H | H | H | 4-COOH | 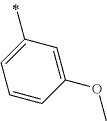 | H | 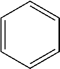 |
| 14 | 5-CF3 | H | H | H | H | H | 4-COOH | 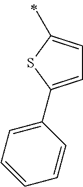 | | 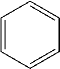 |
| 15 | 5-CF3 | H | H | H | H | H | 4-COOH | 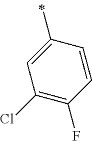 | H | 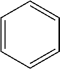 |
| 16 | 5-CF3 | H | H | H | H | H | 4-COOH | 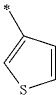 | H | 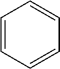 |
| 17 | 5-CF3 | H | H | H | H | H | 4-COOH | 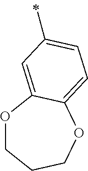 | H | 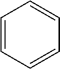 |
| 18 | 5-CF3 | H | H | H | H | H | 4-COOH | 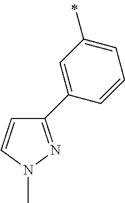 | H | 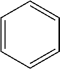 |
| 19 | 5-CF3 | H | H | H | H | H | 4-COOH | 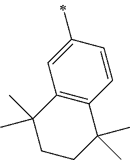 | H | 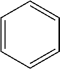 |
| 20 | 5-CF3 | H | H | H | H | H | 4-COOH | 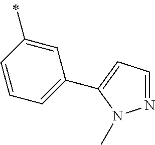 | H | 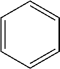 |
| 21 | 5-CF3 | H | H | H | H | H | 4-COOH | 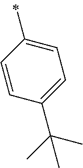 | H | 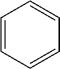 |
| 22 | 5-CF3 | H | H | H | H | H | 4-COOH | 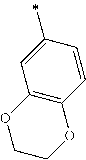 | H | 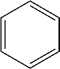 |
| 23 | 5-CF3 | H | H | H | H | H | 4-COOH | 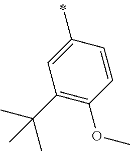 | H | 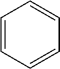 |
| 24 | 5-CF3 | H | H | H | H | H | 4-COOH | 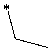 | H | 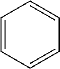 |
| 25 | 5-CF3 | H | H | H | H | H | 4-COOH | 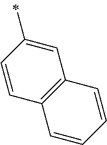 | H | 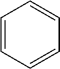 |
| 26 | 5-CF3 | H | H | H | H | H | 4-COOH | 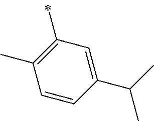 | H | 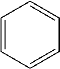 |
| 27 | 5-Cl | H | H | H | H | H | 4-COOH | 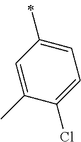 | H | 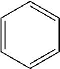 |
| 28 | 5-Cl | H | H | H | H | H | 4-COOH | 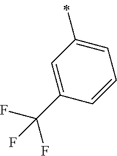 | H | 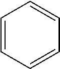 |
| 29 | 5-Cl | H | H | H | H | H | 4-COOH | 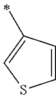 | H | 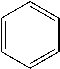 |
| 30 | 5-CF3 | H | H | H | H | OH | 4-COOH | 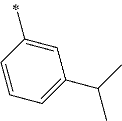 | H | 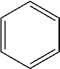 |
| 31 | 5-CF3 | H | H | H | H | H | 4-COOMe | 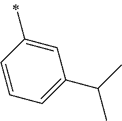 | H | 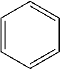 |
| 32 | 5-CF3 | H | H | H | H | H | 4-COOH | 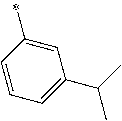 | H | 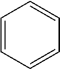 |
| 33 | 5-OCF3 | H | H | H | H | OH | 4-COOMe | 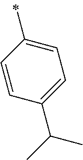 | H | 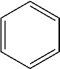 |
| 34 | 5-OCF3 | H | H | H | H | H | 4-COOMe | 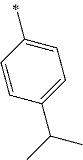 | H | 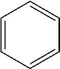 |
| 35 | 5-OCF3 | H | H | H | H | H | 4-COOH | 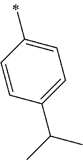 | H | 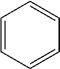 |
| 36 | 5-CF3 | H | H | H | H | OH | 4-COOMe | 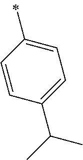 | H | 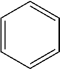 |
| 37 | 5-CF3 | H | H | H | H | OH | 4-COOMe | 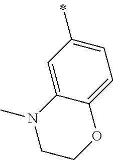 | H | 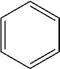 |
| 38 | 5-Cl | H | H | H | H | OH | 4-COOMe | 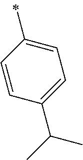 | H | 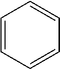 |
| 39 | 5-CF3 | H | H | H | Me | OH | 4-COOMe | 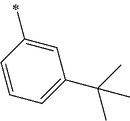 | H | 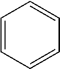 |
| 40 | 5-Cl | H | H | H | H | H | 4-COOMe | 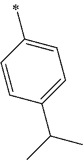 | H | 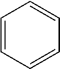 |
| 41 | 5-Cl | H | H | H | H | H | 4-COOH | 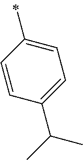 | H | 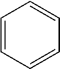 |
| 42 | 5-CF3 | H | H | H | H | OH | 4-COOMe | 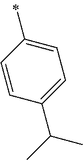 | H | 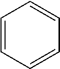 |
| 43 | 5-CF3 | H | H | H | H | H | 4-COOH | 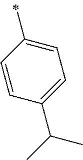 | H | 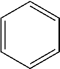 |
| 44 | 5-CF3 | H | H | H | H | H | 4-COOMe | 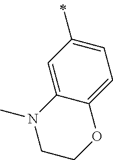 | H | 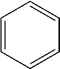 |
| 45 | 5-CF3 | H | H | H | H | H | 4-COOH | 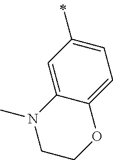 | H | 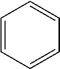 |
| 46 | 5-CF3 | H | H | H | Me | H | 4-COOMe | 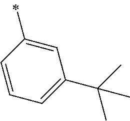 | H | 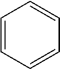 |
| 47 | 5-CF3 | H | H | H | Me | H | 4-COOH | 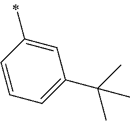 | H | 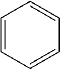 |
| 48 | 5-CF3 | H | H | H | H | H | 4-COOMe | 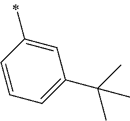 | H | 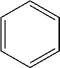 |
| 49 | 5-CF3 | H | H | H | H | H | 4-COOH | 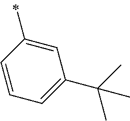 | H | 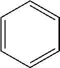 |
| 50 | 5-Cl | H | H | H | H | H | 4-COOMe | 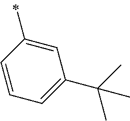 | H | 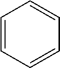 |
| 51 | 5-Cl | H | H | H | H | H | 4-COOH | 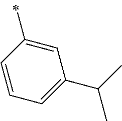 | H | 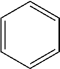 |
| 52 | H | 6- CF3 | H | H | H | H | 4-COOMe | 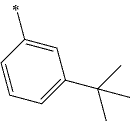 | H | 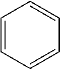 |
| 53 | H | 6- CF3 | H | H | H | H | 4-COOH | 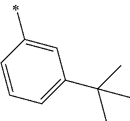 | H | 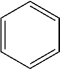 |
| 54 | 4-Cl | H | H | H | H | H | 4-COOMe | 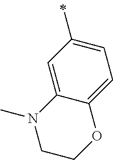 | H | 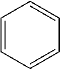 |
| 55 | 4-Cl | H | H | H | H | H | 4-COOH | 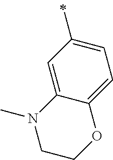 | H | 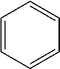 |
| 56 | 5-CF3 | H | 2-OH | H | H | H | 4-COOH | 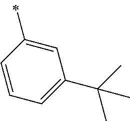 | H | 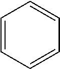 |
| 57 | 5-CF3 | H | H | H | H | H | 4-COOMe | 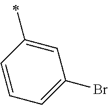 | H | 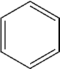 |
| 58 | 5-CF3 | H | H | H | H | H | 4-COOMe | 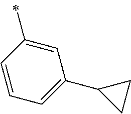 | H | 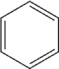 |
| 59 | 5-CF3 | H | H | H | H | H | 4-COOH | 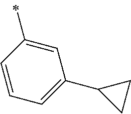 | H | 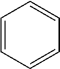 |
| 60 | 5-Cl | H | H | H | H | OH | 4-COOMe | 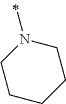 | H | 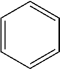 |
| 61 | 5-Cl | H | H | H | H | H | 4-COOMe | 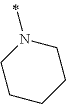 | H | 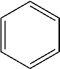 |
| 62 | 5-Cl | H | H | H | H | OH | 3-COOH | 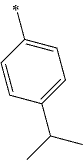 | H | 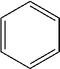 |
| 63 | 5-Cl | H | H | H | H | H | 3-COOH | 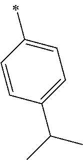 | H | 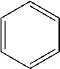 |
| 64 | 5-CF3 | H | H | H | H | OH | 4-COOMe | 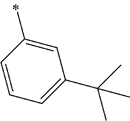 | H | 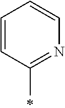 |
| 65 | 5-CF3 | H | H | H | H | H | 4-COOMe | 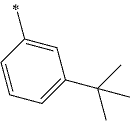 | H | 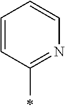 |
| 66 | 5-CF3 | H | H | H | H | H | 4-COOH | 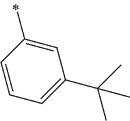 | H | 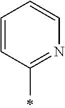 |
| 67 | 5-CF3 | H | 3-F | H | H | H | 4-COOH | 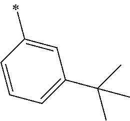 | H | 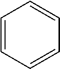 |
| 68 | 5-CF3 | H | 2-OH | H | H | H | 4-COOH | 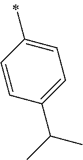 | H | 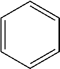 |
| 69 | 5-CF₃ | H | H | H | H | H | 2-COOH | 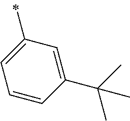 | H | 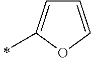 |
| 70 | 5-CF₃ | H | H | H | H | H | 2-COOH | 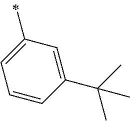 | H | 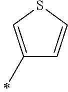 |
| 71 | 5-CF₃ | H | H | H | H | H | 2-COOH | 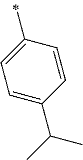 | H | 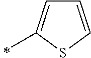 |
| 72 | 5-CF₃ | H | H | H | H | H | 2-COOH | 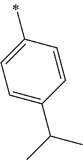 | H | 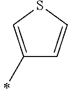 |
| 73 | 5-CF₃ | H | H | H | H | H | 2-COOH | 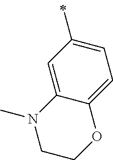 | H | 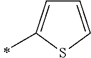 |
| 74 | 5-CF₃ | H | H | H | H | H | 2-COOH | 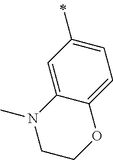 | H | 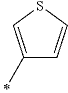 |
| 75 | 5-CF₃ | H | H | H | H | H | 2-COOH | 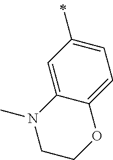 | H | 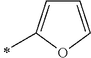 |
| 76 | 5-CF₃ | H | H | H | H | H | 3-COOH | 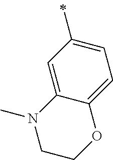 | H | 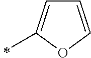 |
| 77 | 5-Cl | H | H | H | H | H | 2-COOH | 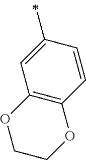 | H | 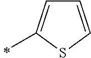 |
| 78 | 5-Cl | H | H | H | H | H | 2-COOH | 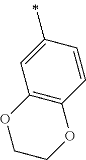 | H | 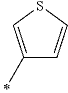 |
| 79 | 5-Cl | H | H | H | H | H | 2-COOH | 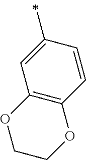 | H | 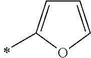 |
| 80 | 5-CF₃ | H | H | H | OH | H | 2-COOMe | 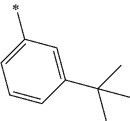 | CH₃ | 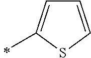 |
| 81 | 5-CF₃ | H | H | H | OH | H | 4-COOEt | 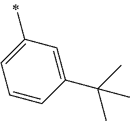 | CH₃ | 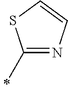 |
| 82 | 5-CF₃ | H | H | H | OH | H | 2-COOEt | 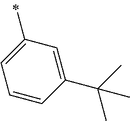 | H | 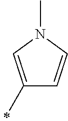 |
| 83 | 5-CF₃ | H | H | H | H | H | 2-COOMe | 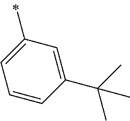 | CH₃ | 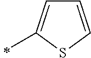 |
| 84 | 5-CF₃ | H | H | H | H | H | 4-COOEt | 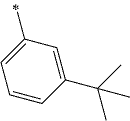 | CH₃ | 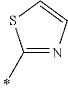 |
| 85 | 5-CF₃ | H | H | H | H | H | 2-COOH | 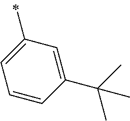 | CH₃ | 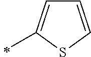 |
| 86 | 5-CF₃ | H | H | H | H | H | 4-COOH | 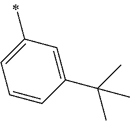 | CH₃ | 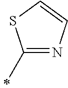 |
| 87 | 5-CF₃ | H | CH₃ | H | H | H | 2-COOH | 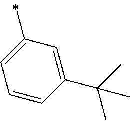 | H | 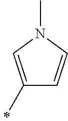 |
| 88 | 5-CF₃ | H | H | H | H | H | 4-COOEt | 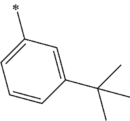 | H | 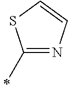 |
| 89 | 5-CF₃ | H | H | H | H | H | 4-COOH | 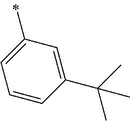 | H | 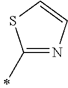 |
| 90 | 5-CF₃ | H | H | H | OH | H | 2-COOMe | 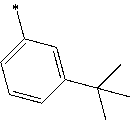 | H | 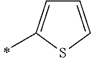 |
| 91 | 5-CF₃ | H | H | H | OH | H | 2-COOH | 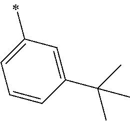 | H | 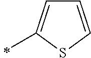 |
| 92 | 5-CF₃ | H | H | H | H | H | 2-COOH | 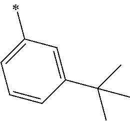 | H | 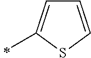 |
| 93 | 5-CF₃ | H | H | H | O | | 2-COOMe | 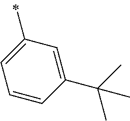 | H | 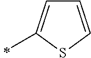 |
| 94 | 5-CF₃ | H | H | H | O | | 2-COOH | 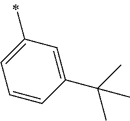 | H | 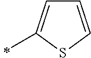 |
| 95 | 5-CF₃ | H | H | H | CH₃ | OH | 2-COOH | 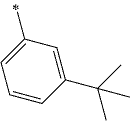 | H | 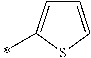 |
| 96 | 5-CF₃ | H | H | H | CH₂ | | 2-COOH | 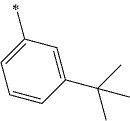 | H | 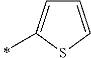 |
| 97 | 5-Cl | H | H | 2- OMe | H | H | 4-COOMe | 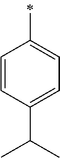 | H | 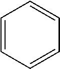 |
| 98 | 5-Cl | H | H | 2- OMe | H | H | 4-COOH | 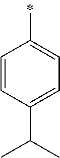 | H | 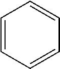 |
| 99 | 5-Cl | H | H | 2- OH | H | H | 4-COOH | 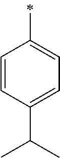 | H | 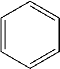 |
| 100 | 5-Cl | H | H | H | CH₃ | OH | 4-COOMe | 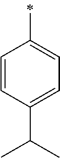 | H | 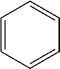 |
| 101 | 5-Cl | H | H | H | CH₂ | | 4-COOMe | 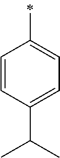 | H | 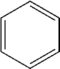 |
| 102 | 5-Cl | H | H | H | CH₂ | | 4-COOH | 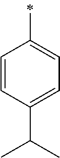 | H | 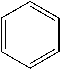 |
| 103 | 5- C(CH₃)₃ | H | H | H | H | OH | 4-COOMe | 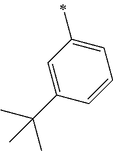 | H | 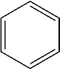 |
| 104 | 5-Br | H | H | H | H | OH | 4-COOMe | 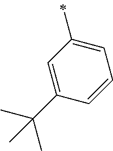 | H | 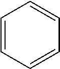 |
| 105 | 5-CF₃ | 6-F | H | H | H | OH | 4-COOMe | 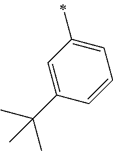 | H | 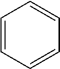 |
| 106 | 5-CH₃ | H | H | H | H | OH | 4-COOMe | 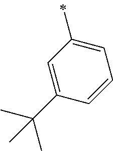 | H | 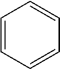 |
| 107 | 4-Cl | 5-Cl | H | H | H | OH | 4-COOMe | 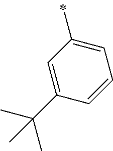 | H | 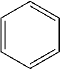 |
| 108 | 5- C(CH₃)₃ | H | H | H | H | H | 4-COOMe | 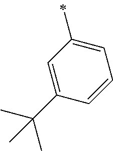 | H | 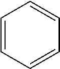 |
| 109 | 5-Br | H | H | H | H | H | 4-COOMe | 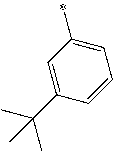 | H | 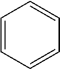 |
| 110 | 5-CF₃ | 6-F | H | H | H | H | 4-COOMe | 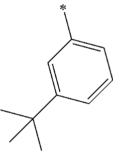 | H | 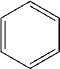 |
| 111 | 5-CH₃ | H | H | H | H | H | 4-COOMe | 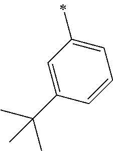 | H | 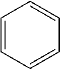 |
| 112 | 4-Cl | 5-Cl | H | H | H | H | 4-COOMe | 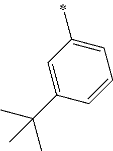 | H | 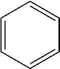 |
| 113 | H | 7-F | H | H | H | H | 4-COOMe | 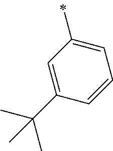 | H | 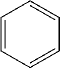 |
| 114 | 5- C(CH₃)₃ | H | H | H | H | H | 4-COOH | 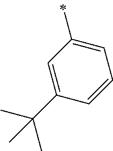 | H | 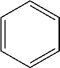 |
| 115 | 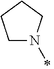 | H | H | H | H | H | 4-COOMe | 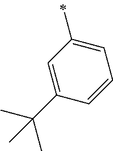 | H | 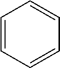 |
| 116 | 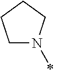 | H | H | H | H | H | 4-COOH | 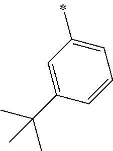 | H | 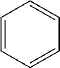 |
| 117 | 5-CF₃ | 6-F | H | H | H | H | 4-COOH | 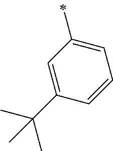 | H | 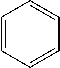 |
| 118 | 5-CH₃ | H | H | H | H | H | 4-COOH | 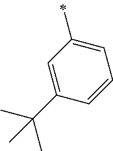 | H | 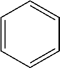 |
| 119 | 4-Cl | 5-Cl | H | H | H | H | 4-COOH | 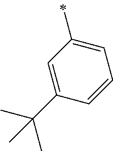 | H | 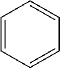 |
| 120 | 5-CF₃ | H | H | H | H | H | 4-COOH | 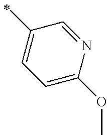 | H | 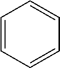 |
| 121 | 5-CF₃ | H | H | H | H | H | 4-COOH | 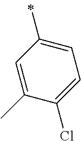 | H | 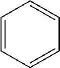 |
| 122 | 5-CF₃ | H | H | H | H | H | 4-COOH | 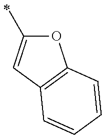 | H | 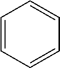 |
| 123 | 5-CF₃ | H | H | H | H | H | 4-COOH | 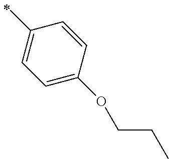 | H | 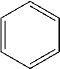 |
| 124 | 5-CF₃ | H | H | H | H | H | 4-COOH | 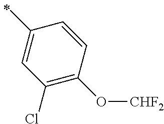 | H | 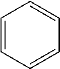 |
| 125 | 5-CF₃ | H | H | H | H | H | 4-COOH | 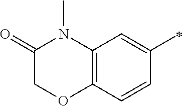 | H | 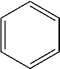 |
| 126 | 5-CF₃ | H | H | H | H | H | 4-COOH | 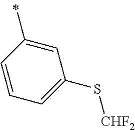 | H | 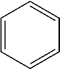 |
| 127 | 5-CF₃ | H | H | H | H | H | 4-COOH | 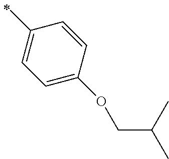 | H | 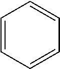 |
| 128 | 5-CF₃ | H | H | H | H | H | 4-COOH | 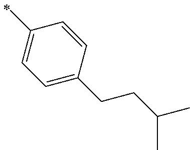 | H | 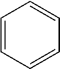 |
| 129 | 5-CF₃ | H | H | H | H | H | 4-COOH | 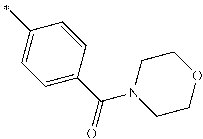 | H | 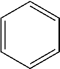 |
| 130 | 5-CF₃ | H | H | H | H | H | 4-COOH | 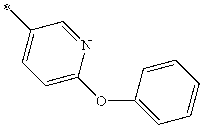 | H | 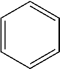 |
| 131 | 5-CF₃ | H | H | H | H | H | 4-COOH | 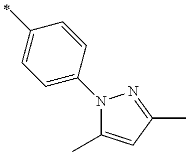 | H | 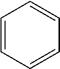 |
| 132 | 5-CF₃ | H | H | H | H | H | 4-COOH | 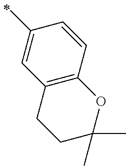 | H | 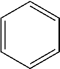 |
| 133 | 5-CF₃ | H | H | H | H | H | 4-COOH | 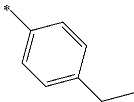 | H | 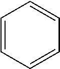 |
| 134 | 5-CF₃ | H | H | H | H | H | 4-COOH | 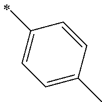 | H | 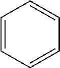 |
| 135 | 5-CF₃ | H | H | H | H | H | 4-COOH | 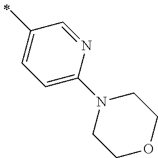 | H | 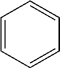 |
| 136 | 5-CF₃ | H | H | H | H | H | 4-COOH | 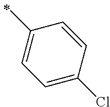 | H | 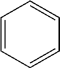 |
| 137 | 5-CF₃ | H | H | H | H | H | 4-COOH | 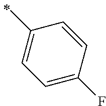 | H | 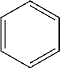 |
| 138 | 5-CF₃ | H | H | H | H | H | 4-COOH | 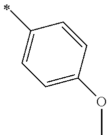 | H | 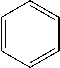 |
| 139 | 5-CF₃ | H | H | H | H | H | 4-COOH | 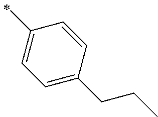 | H | 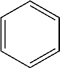 |
| 140 | 5-CF₃ | H | H | H | H | H | 4-COOH | 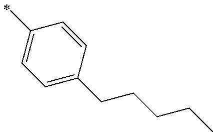 | H | 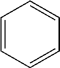 |
| 141 | 5-CF₃ | H | H | H | H | H | 4-COOH | 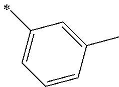 | H | 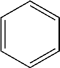 |
| 142 | 5-CF₃ | H | H | H | H | H | 4-COOH | 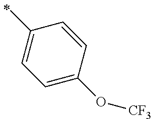 | H | 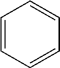 |
| 143 | 5-CF₃ | H | H | H | H | H | 4-COOH | 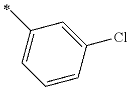 | H | 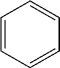 |
| 144 | 5-CF₃ | H | H | H | H | H | 4-COOH | 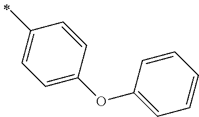 | H | 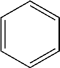 |
| 145 | 5-CF₃ | H | H | H | H | H | 4-COOH | 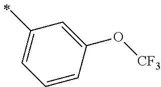 | H | 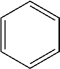 |
| 146 | 5-CF₃ | H | H | H | H | H | 4-COOH | 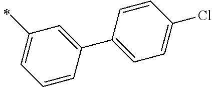 | H | 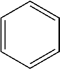 |
| 147 | 5-CF₃ | H | H | H | H | H | 4-COOH | 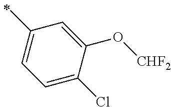 | H | 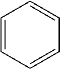 |
| 148 | 5-CF₃ | H | H | H | H | H | 4-COOMe | 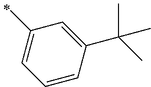 | F | 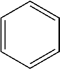 |
| 149 | 5-CF₃ | H | H | H | H | H | 4-COOH | 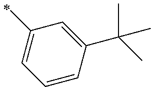 | F | 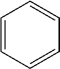 |
| 150 | 5-Cl | H | H | H | H | H | 4-COOH | 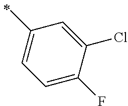 | H | 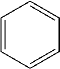 |
| 151 | 5-Cl | H | H | H | H | H | 4-COOH | 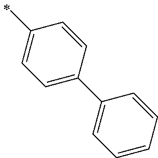 | H | 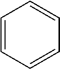 |
| 152 | 5-Cl | H | H | H | H | H | 4-COOH | 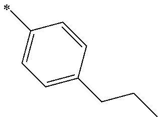 | H | 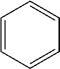 |
| 153 | 5-Cl | H | H | H | H | H | 4-COOH | 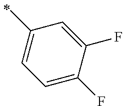 | H | 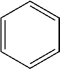 |
| 154 | 5-Cl | H | H | H | H | H | 4-COOH | 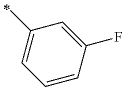 | H | 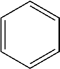 |
| 155 | 5-Cl | H | H | H | H | H | 4-COOH | 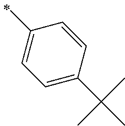 | H | 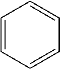 |
| 156 | 5-Cl | H | H | H | H | H | 4-COOH | 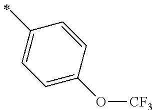 | H | 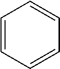 |
| 157 | 5-Cl | H | H | H | H | H | 4-COOH | 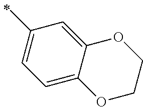 | H | 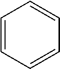 |
| 158 | 5-Cl | H | H | H | H | H | 4-COOH | 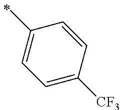 | H | 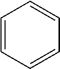 |
| 159 | 5-Cl | H | H | H | H | H | 4-COOH | 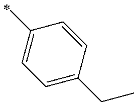 | H | 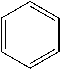 |
| 160 | 5-Cl | H | H | H | H | H | 4-COOH | 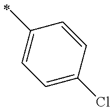 | H | 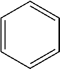 |
| 161 | 5-Cl | H | H | H | H | H | 4-COOH | 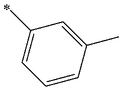 | H | 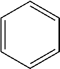 |
| 162 | 5-Cl | H | H | H | H | H | 4-COOH | 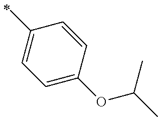 | H | 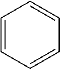 |
| 163 | 5-Cl | H | H | H | H | H | 4-COOH | 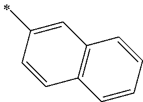 | H | 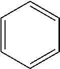 |
| 164 | 5-Cl | H | H | H | H | H | 4-COOH | 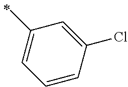 | H | 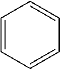 |
| 165 | 5-Cl | H | H | H | H | H | 4-COOH | 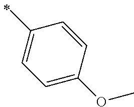 | H | 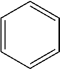 |
| 166 | 5-Cl | H | H | H | H | H | 4-COOH | 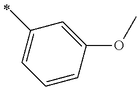 | H | 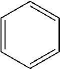 |
| 167 | 5-Cl | H | H | H | H | H | 4-COOH | 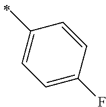 | H | 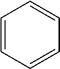 |
| 168 | 5-Cl | H | H | H | H | H | 4-COOH | 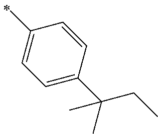 | H | 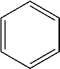 |
| 169 | 5-Cl | H | H | H | H | H | 4-COOH | 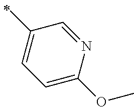 | H | 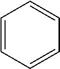 |
| 170 | 5-Cl | H | H | H | H | H | 4-COOH | 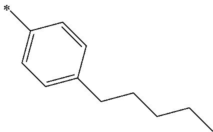 | H | 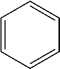 |
| 171 | 5-Cl | H | H | H | H | H | 4-COOH | 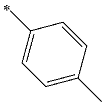 | H | 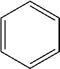 |
| 172 | 5-Cl | H | H | H | H | H | 4-COOH | 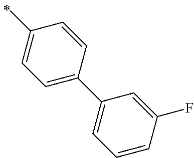 | H | 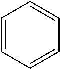 |
| 173 | 5-Cl | H | H | H | H | H | 4-COOH | 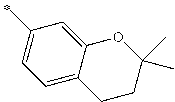 | H | 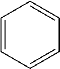 |
| 174 | 5-Cl | H | H | H | H | H | 4-COOH | 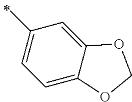 | H | 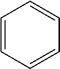 |
| 175 | 5-Cl | H | H | H | H | H | 4-COOH | 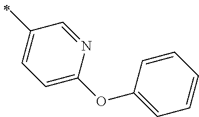 | H | 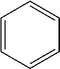 |
| 176 | 5-Cl | H | H | H | H | H | 4-COOH | CH₂CH₃ | H | 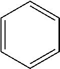 |
| 177 | 5-Cl | H | H | H | H | H | 4-COOH | 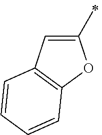 | H | 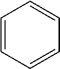 |
| 178 | 5-Cl | H | H | H | H | H | 4-COOH | 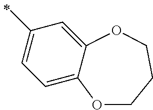 | H | 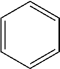 |
| 179 | 5-Cl | H | H | H | H | H | 4-COOH | 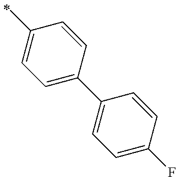 | H | 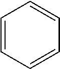 |
| 180 | 5-Cl | H | H | H | H | H | 4-COOMe | CH₃ | H | 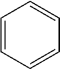 |
| 181 | 5-Cl | H | H | H | H | H | 4-COOH | CH₃ | H | 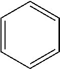 |
| 182 | 5-CF₃ | H | H | H | H | H | 4-COOMe | 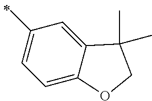 | H | 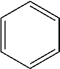 |
| 183 | 5-CF₃ | H | H | H | H | H | 4-COOH | 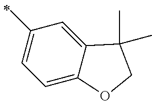 | H | 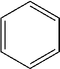 |
| 184 | 5-CF₃ | H | H | H | OH | H | 3-COOMe | 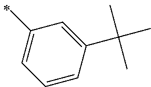 | CH₃ | 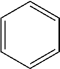 |
| 185 | 5-CF₃ | H | H | H | H | H | 3-OOMe | 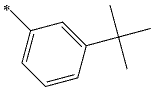 | CH₃ | 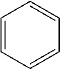 |
| 186 | 5-CF₃ | H | H | H | H | H | 3-COOH | 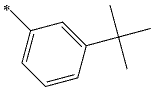 | CH₃ | 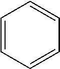 |
| 187 | 5-CF₃ | H | H | H | OH | H | 4-COOMe | 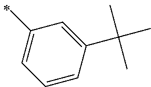 | CH₃ | 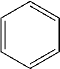 |
| 188 | 5-CF₃ | H | H | H | H | H | 4-COOMe | 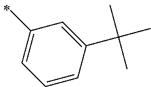 | CH₃ | 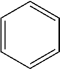 |
| 189 | 5-CF₃ | H | H | H | H | H | 4-COOH | 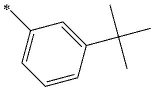 | CH₃ | 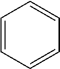 |
| 190 | 5-CF₃ | H | H | H | H | H | 5-COOMe | 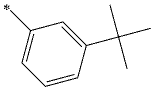 | H | 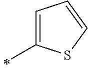 |
| 191 | 5-CF₃ | H | H | H | OH | H | 4-COOMe | 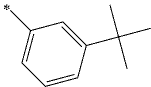 | H | 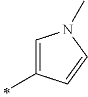 |
| 192 | 5-CF₃ | H | H | H | H | H | 4-COOMe | 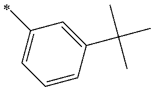 | H | 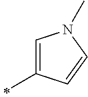 |
| 193 | H | H | H | H | OH | H | 4-COOMe | 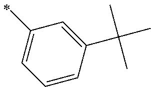 | H | 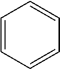 |
| 194 | H | H | H | H | H | H | 4-COOMe | 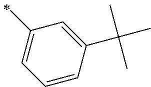 | H | 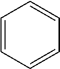 |
| 195 | H | H | H | H | H | H | 4-COOH | 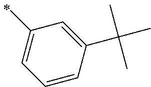 | H | 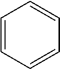 |
| 196 | 5-CF₃ | H | 2- CH₃ | 5-F | H | H | 4-COOH | 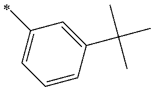 | H | 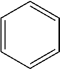 |
| 197 | 5-CF₃ | H | H | 3- CH₃ | H | H | 4-COOH | 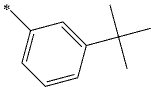 | H | 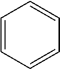 |
| 198 | 5-CF₃ | H | H | H | H | H | 4-SO₂NH₂ | 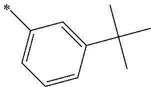 | H | 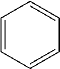 |
| 199 | 5-CF₃ | H | H | 4-F | H | H | 3-COOH | 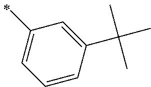 | H | 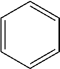 |
| 200 | 5-CF₃ | H | H | 5-F | H | H | 3-COOH | 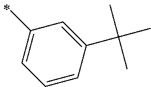 | H | 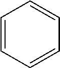 |
| 201 | 5-CF₃ | H | H | 6-F | H | H | 3-COOH | 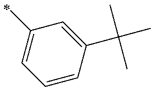 | H | 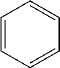 |
| 202 | 5-CF₃ | H | H | 6- OMe | H | H | 3-COOH | 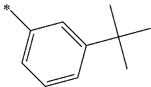 | H | 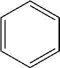 |
| 203 | 5-CF₃ | H | 4-Cl | 6-F | H | H | 3-COOH | 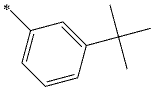 | H | 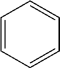 |
| 204 | 5-CF₃ | H | H | H | H | H | 3-COOH | 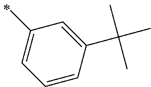 | H | 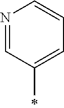 |
| 205 | 5-CF₃ | H | 3-Cl | H | H | H | 4-COOH | 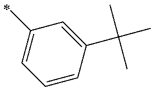 | H | 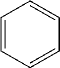 |
| 206 | 5-CF₃ | H | 2-Cl | H | H | H | 4-COOH | 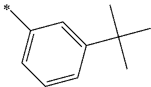 | H | 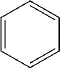 |
| 207 | 5-CF₃ | H | 2- OMe | H | H | H | 3-COOH | 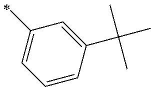 | H | 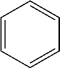 |
| 208 | 5-CF₃ | H | H | 4- OMe | H | H | 3-COOH | 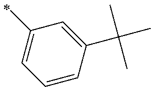 | H | 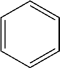 |
| 209 | 5-CF₃ | H | 2-Cl | H | H | H | 4-COOH | 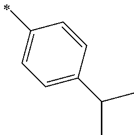 | H | 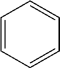 |
| 210 | 5-CF₃ | H | H | 6-F | H | H | 3-COOH | 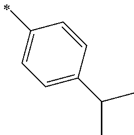 | H | 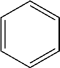 |
| 211 | 5-CF₃ | H | 2-Cl | H | H | H | 4-COOH | 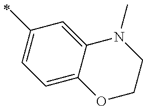 | H | 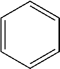 |
| 212 | 5-CF₃ | H | H | 6-F | H | H | 3-COOH | 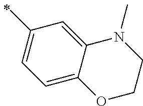 | H | 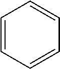 |
| 213 | 5-Cl | H | H | H | H | H | 3-COOH | 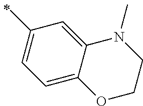 | H | 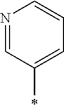 |
| 214 | 5-Cl | H | H | 4-F | H | H | 3-COOH | 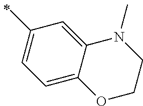 | H | 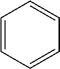 |
| 215 | 5-CF₃ | H | H | H | OH | H | 4-COOMe | 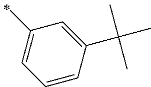 | H | 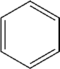 |
| 216 | 5-CF₃ | H | H | H | F | H | 4-COOMe | 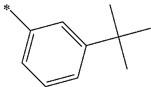 | H | 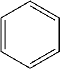 |
| 217 | 5-CF₃ | H | H | H | F | H | 4-COOH | 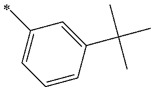 | H | 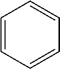 |
| 218 | 5-CF₃ | H | H | H | OH | H | 4-COOH | 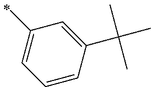 | H | 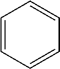 |
| 219 | 5-CF₃ | H | H | H | O | | 4-COOH | 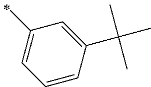 | H | 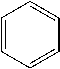 |
| 220 | 5-CF₃ | H | H | H | H | H | 4-CN | 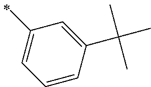 | H | 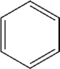 |
| 221 | 5-CF₃ | H | H | H | H | H | 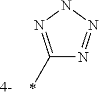 | 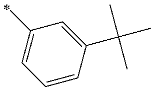 | H | 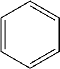 |
| 222 | 5-CF₃ | H | H | H | H | H | 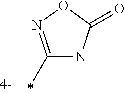 | 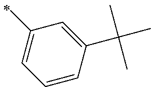 | H | 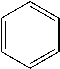 |
| 223 | 5-CF₃ | H | H | H | H | | 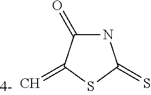 | 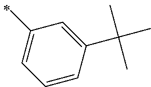 | H | 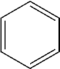 |
| 224 | 5-CF₃ | H | H | H | H | H | 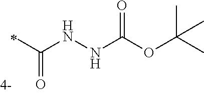 | 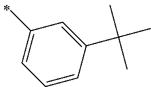 | H | 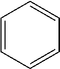 |
| 225 | 5-CF₃ | H | H | H | H | H | 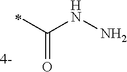 | 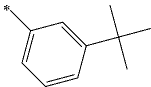 | H | 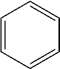 |
| 226 | 5-CF₃ | H | H | H | H | H | 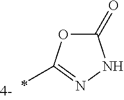 | 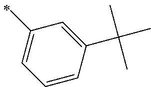 | H | 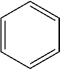 |
| 227 | 5-CF₃ | H | H | H | H | H | 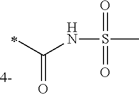 | 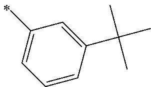 | H | 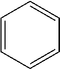 |
| 228 | 5-CF₃ | H | H | H | H | H | 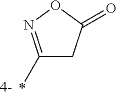 | 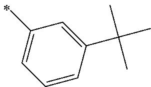 | H | 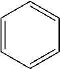 |
| 229 | 5-CF₃ | H | H | H | H | H | 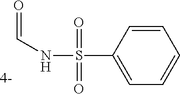 | 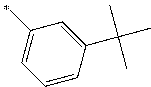 | H | 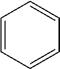 |
| 230 | 5-CF₃ | H | H | H | H | H | 4-COOMe | 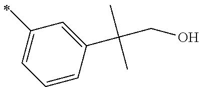 | H | 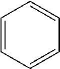 |
| 231 | 5-CF₃ | H | H | H | H | H | 4-COOH | 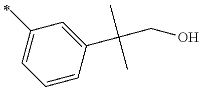 | H | 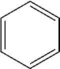 |

### Pharmacological Activity

The compounds of the invention were submitted to biological assays to evaluate their potential for treating or inhibiting certain neurodegenerative pathologies.

First, the capacity of the compounds according to the invention for acting as an activator of the heterodimers formed by the NURR-1 nuclear receptor and the RXR nuclear receptors was measured by an in vitro assay.

A transactivation assay was used as a primary screening assay. Cos-7 cells were co-transfected with a plasmid expressing a chimera of the human receptor NURR-1-Gal4, a plasmid expressing the human receptor RXR (RXRα or RXRγ receptor) and a reporter plasmid 5Gal4pGL3-TK-Luc. The transfections were performed by means of a chemical agent (Jet PEI).

The transfected cells were distributed in 384-well plates and left resting for 24 hours.

After 24 hours, the culture medium was changed. The products according to the invention were added (final concentration between 10⁻⁴ and 3·10⁻¹⁰ M) to the culture medium. After overnight incubation, luciferase expression was measured after addition of “SteadyGlo” according to the manufacturer's instructions (Promega).

4-[[6-Methyl-2-phenyl-5-(2-prop enyl)-4-pyrimidinyl]amino]-benzoic acid (called XCT0135908, which was described in the article by Wallen-Mackenzie et al. published in Genes & Development 17, pages 3036-3047) at 2·10⁻⁵ M (RXR agonist) was used as reference.

The levels of induction (designated by “efficiency”) were calculated relative to the baseline activity of each heterodimer. The results were expressed as a percentage of the level of induction relative to the level of induction obtained with the reference (the level of induction of the reference is arbitrarily taken to be 100%).

The compounds according to the invention show a degree of induction of up to 150% (NURR1/RXRα) and 152% (NURR1/RXRγ) and EC50 values of up to 3 nM (NURR1/RXRα) and 8 nM (NURR1/RXRγ).

As an example, among the compounds according to the invention, the following comparative results are obtained, expressed as a percentage relative to an NURR-1/RXR activator reference compound (XCT0135908):

| | hNurr1_RXRγFL | | hNurr1_RXRαFL | |
|---|---|---|---|---|
| Example | EC₅₀ (nM) | Eff (%) | | EC₅₀ (nM) |
| 8 | 99 | 74 | 101 | 96 |
| 40 | 2108 | 75 | 560 | 96 |
| 56 | 417 | 51 | 1161 | 82 |
| 51 | 1130 | 69 | 801 | 74 |
| 49 | 110 | 71 | 48 | 73 |
| 43 | 616 | 79 | 291 | 73 |
| 45 | 201 | 50 | 97 | 70 |
| 59 | 297 | 31 | 274 | 70 |
| 13 | 528 | 36 | 207 | 69 |
| 14 | 778 | 30 | 396 | 69 |
| 32 | 219 | 62 | 103 | 68 |
| 15 | 746 | 54 | 372 | 66 |
| 16 | 628 | 45 | 617 | 66 |
| 1 | 718 | 60 | 412 | 65 |
| 10 | 943 | 56 | 406 | 63 |
| 2 | 724 | 43 | 657 | 63 |
| 18 | 668 | 34 | 407 | 60 |
| 12 | 148 | 45 | 113 | 54 |
| 20 | 617 | 44 | 295 | 54 |
| 68 | 553 | 43 | 343 | 51 |
| 25 | 864 | 33 | 496 | 47 |
| 21 | 240 | 35 | 121 | 46 |
| 22 | 346 | 43 | 207 | 45 |
| 28 | 443 | 67 | 332 | 78 |
| 66 | 110 | 79 | 71 | 80 |
| 67 | 91 | 75 | 28 | 73 |
| 85 | 115 | 73 | 53 | 87 |
| 69 | 15 | 72 | 9 | 79 |
| 70 | 8 | 91 | 5 | 94 |
| 73 | 66 | 89 | 37 | 94 |
| 89 | 611 | 62 | 265 | 77 |
| 86 | 1479 | 34 | 490 | 56 |
| 71 | 95 | 82 | 47 | 61 |
| 77 | 457 | 38 | 224 | 74 |
| 91 | nd | nd | 72 | 100 |
| 94 | nd | nd | 1240 | 106 |
| 74 | nd | nd | 40 | 150 |
| 75 | nd | nd | 66 | 114 |
| 76 | nd | nd | 63 | 138 |
| 78 | nd | nd | 331 | 116 |
| 79 | nd | nd | 724 | 147 |
| 72 | nd | nd | 72 | 131 |
| 95 | nd | nd | 209 | 99 |
| 87 | nd | nd | 30 | 123 |
| 96 | nd | nd | 339 | 54 |
| 98 | >10 000 | 8 | 2122 | 64 |
| 99 | 989 | 42 | 569 | 50 |
| 102 | 676 | 27 | 363 | 49 |
| 108 | 646 | 77 | 157 | 110 |
| 110 | 63 | 95 | 15 | 92 |
| 114 | 145 | 76 | 63 | 109 |
| 116 | 1445 | 50 | 871 | 74 |
| 117 | 91 | 73 | 39 | 84 |
| 118 | 473 | 65 | 279 | 75 |
| 119 | 266 | 82 | 65 | 71 |
| 113 | 1862 | 97 | >10 000 | 103 |
| 120 | 661 | 49 | 202 | 68 |
| 121 | 433 | 56 | 192 | 65 |
| 122 | 680 | 54 | 353 | 62 |
| 123 | 646 | 36 | 217 | 60 |
| 124 | 697 | 44 | 349 | 59 |
| 125 | 724 | 101 | 808 | 61 |
| 126 | 141 | 32 | 122 | 44 |
| 127 | 575 | 45 | 263 | 48 |
| 128 | 1585 | 36 | 337 | 44 |
| 129 | nd | nd | 804 | 41 |
| 130 | 1698 | 22 | 347 | 30 |
| 131 | 1479 | 21 | 603 | 30 |
| 132 | 352 | 28 | 221 | 45 |
| 133 | 304 | 38 | 177 | 49 |
| 134 | 552 | 36 | 212 | 50 |
| 135 | 917 | 19 | 643 | 31 |
| 136 | >10 000 | 25 | 275 | 35 |
| 137 | 730 | 18 | 529 | 37 |
| 138 | 583 | 28 | 344 | 41 |
| 139 | 344 | 43 | 202 | 46 |
| 140 | 387 | 22 | 368 | 46 |
| 141 | 364 | 30 | 285 | 41 |
| 142 | 816 | 19 | 459 | 38 |
| 143 | 425 | 18 | 339 | 33 |
| 144 | >10 000 | 19 | 839 | 30 |
| 145 | 638 | 21 | 359 | 44 |
| 146 | 1177 | 24 | 619 | 43 |
| 147 | 1095 | 30 | 825 | 49 |
| 148 | 121 | 101 | 62 | 119 |
| 149 | 225 | 80 | 94 | 98 |
| 150 | 501 | 96 | 299 | 103 |
| 151 | >10 000 | 17 | 427 | 80 |
| 152 | 858 | 75 | 269 | 87 |
| 153 | 1391 | 63 | 851 | 85 |
| 154 | 1365 | 66 | 472 | 68 |
| 155 | 468 | 59 | 262 | 69 |
| 156 | 1303 | 43 | 638 | 69 |
| 157 | 838 | 64 | 267 | 67 |
| 158 | 1259 | 53 | 504 | 66 |
| 159 | 785 | 45 | 319 | 66 |
| 160 | 1391 | 55 | 462 | 59 |
| 161 | 584 | 40 | 460 | 59 |
| 162 | 1549 | 40 | 692 | 56 |
| 163 | 933 | 34 | 427 | 53 |
| 164 | 851 | 47 | 753 | 53 |
| 165 | 2929 | 37 | 1142 | 51 |
| 166 | 1072 | 27 | 681 | 50 |
| 167 | 2436 | 40 | 1148 | 48 |
| 168 | 501 | 52 | 422 | 48 |
| 169 | 2493 | 28 | 1012 | 46 |
| 170 | >10 000 | 26 | 1738 | 42 |
| 171 | 944 | 31 | 510 | 40 |
| 172 | 567 | 47 | 117 | 82 |
| 173 | 498 | 73 | 145 | 78 |
| 174 | 813 | 37 | 593 | 47 |
| 175 | >10 000 | 14 | 331 | 38 |
| 176 | >10 000 | 21 | 3388 | 33 |
| 177 | >10 000 | 17 | >10 000 | 39 |
| 178 | 1196 | 36 | 1029 | 45 |
| 179 | >10 000 | 26 | 331 | 62 |
| 181 | 4074 | 152 | 1365 | 111 |
| 182 | 154 | 85 | 55 | 83 |
| 183 | 95 | 91 | 47 | 80 |
| 186 | 288 | 68 | 124 | 93 |
| 189 | 263 | 90 | 120 | 83 |
| 190 | 13 | 107 | 0.2 | 97 |
| 192 | >10 000 | 58 | 1122 | 85 |
| 194 | 316 | 36 | 344 | 42 |
| 195 | 1169 | 56 | 629 | 57 |
| 196 | 479 | 91 | 240 | 90 |
| 197 | 592 | 52 | 278 | 68 |
| 198 | >10 000 | 86 | 7943 | 84 |
| 199 | nd | nd | 22 | 118 |
| 200 | nd | nd | 92 | 93 |
| 201 | nd | nd | 3 | 96 |
| 202 | nd | nd | 19 | 104 |
| 203 | nd | nd | 85 | 83 |
| 204 | 195 | 93 | 107 | 102 |
| 205 | nd | nd | 98 | 75 |
| 206 | 363 | 85 | 174 | 71 |
| 207 | nd | nd | 513 | 48 |
| 208 | nd | nd | 240 | 31 |
| 209 | 741 | 51 | 333 | 69 |
| 210 | 81 | 68 | 69 | 70 |
| 211 | 465 | 56 | 385 | 83 |
| 212 | 40 | 81 | 19 | 80 |
| 213 | >10 000 | 31 | 3003 | 45 |
| 214 | 804 | 55 | 681 | 75 |
| 217 | 355 | 80 | 149 | 83 |
| 219 | 3199 | 32 | 1132 | 57 |
| 221 | 162 | 57 | 191 | 66 |
| 222 | 676 | 55 | 367 | 62 |
| 223 | 3388 | 30 | 1318 | 72 |
| 226 | >10 000 | 70 | 465 | 82 |
| 227 | 316 | 86 | 219 | 73 |
| 228 | 335 | 63 | 113 | 90 |
| 229 | 303 | 70 | 157 | 60 |
| 231 | 164 | 62 | 65 | 81 |

Eff denotes: efficacy in % relative to the reference XCT0135908
nd: not determined

A first series of in vivo assays were conducted with some compounds according to the invention, for the purpose of determining their plasma and cerebral pharmacokinetic profile in the male C57B16 mouse and thus verify that the compounds were capable of crossing the blood-brain barrier. The following procedure was used.

Male C57B16 mice (25-30 g) obtained from the Janvier establishment, Le Genest-St-Isle, France were used for this study (12 mice per dose). The animals were fed with standard rodent feed (Purina Mills, St. Louis, Mo.), placed in cages and subjected to light/darkness cycles of 12 h/12 h, the temperature of the room being maintained at 22±2° C. and the humidity at 55±10%. The mice were not fasting before administration. Water was supplied ad libitum throughout the study. The assayed compound was administered orally at 10 mg/kg. For oral administration at 10 mg/kg, the animals were force-fed with 10 mL/kg of a suspension of the assayed compound, prepared in methylcellulose 400 cp 1%. The animals were euthanized by anaesthesia at intervals of 15 min, 30 min, 1 h, 3 h, 6 h and 8 h after force-feeding. At each interval, blood was collected and the brain was removed from each euthanized animal. 1 mL of blood collected in 1.5 mL tubes containing 20 μL of evaporated anticoagulant (solution of sodium heparinate at 1000 IU/mL) was centrifuged at 4500 g for 3 min, obtaining about 400 μL of plasma. The plasma was distributed in 2 aliquots of 200 μL which were stored at −20° C. until extraction by protein precipitation and then analysis by liquid chromatography coupled with tandem mass spectrometry (LC-MS/MS) for quantification of the assayed compound. The brains were immersed in liquid nitrogen directly after removal, then stored at −20° C. for analysis. The brains were then ground in the presence of aqueous/organic solvent mixture to obtain a homogenate. These homogenates were then centrifuged and the assayed compound was extracted from the supernatant by liquid-liquid extraction, then quantified by LC-MS/MS.

The pharmacokinetic parameters were determined according to a non-compartmental approach using Excel. The area under curve (AUC₀₋ₜ) was determined by the linear trapezoidal method. This method allows estimation of the integral of the concentrations over an interval of time (AUC0-t) and is based on the sum of the areas of the trapeziums delimited by the concentrations measured at the time of sampling (example AUC0-8 h=AUC0-0.25 h+AUC0.25 h-0.5 h+AUC0.5 h-t+AUCt-8 h). The penetration of the compounds through the blood-brain barrier is assessed from the ratio of the AUC measured in the brain to that measured in plasma. As an example, with the compounds of examples 32 and 49, the following results were obtained:

PK data after oral administration:
10 mg/kg in the mouse

| | AUC_{brain} | Ratio |
|---|---|---|
| Compound | (ng · h/mL) | AUC_{brain/}AUCₚₗₐₛₘₐ |
| Example 32 | 3763 | 0.8 |
| Example 49 | 10491 | 0.8 |

The results obtained show that these two compounds penetrate the blood-brain barrier satisfactorily.

A second series of in vivo assays were conducted with the compounds of the invention, to verify that the molecules do indeed possess the expected neuroprotective effect.

The compounds of examples 32 and 49 were assayed on a model of mice treated with 1-methyl-4-phenyl-1,2,3,6-tetrahydropyridine (MPTP) in order to confirm their potential activities. MPTP is a neurotoxin which causes permanent symptoms of Parkinson's disease by destroying certain neurons in the substantia nigra of the brain. The following procedure was used.

Male C57BL6/J mice aged 10-12 weeks at the start of the studies were distributed in groups of 8 animals. The compounds were administered orally, twice a day for a total of 11 days. Administration began 3 days before the treatment with the MPTP toxin at 25 mg/kg. MPTP was administered once a day by intraperitoneal injection for 5 days. Administration of the assayed compounds was continued for 3 days after the treatment with MPTP. One group of mice received the carrier only (0.5% solution of methylcellulose). The animals were euthanized after the last force-feeding and the striatum was removed. Dopamine was extracted from the striatum and the amount of dopamine (DA) expressed in ng per g of striatum (mean±SEM) was measured by high-performance liquid chromatography (HPLC) with electrochemical detection.

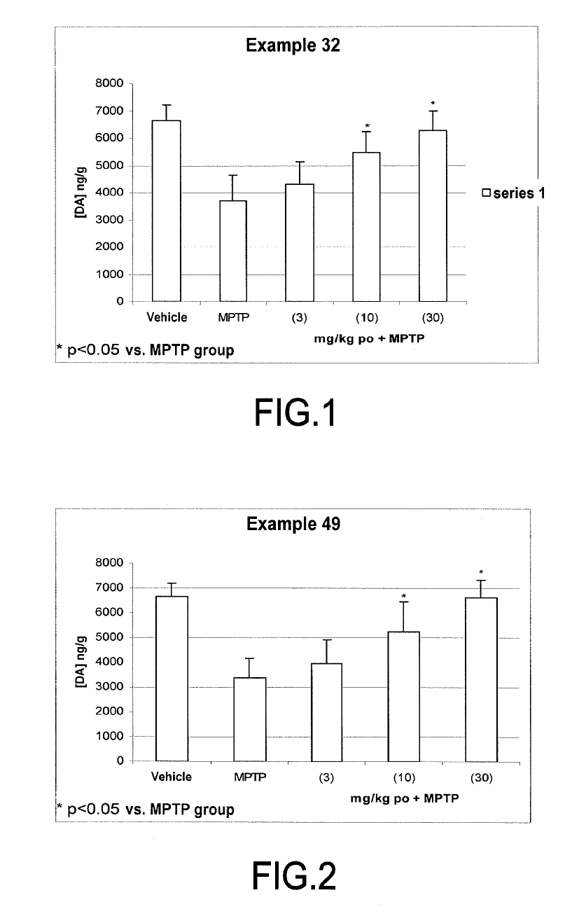

The results obtained are shown in the appended FIGS. 1 and 2. These results show that the administration of MPTP causes a characteristic decrease in the level of dopamine in the striatum and that the compounds of examples 32 and 49 cause a dose-dependent decrease in the action of MPTP, a toxin which causes a parkinsonian syndrome. Thus, a significant effect is observed at doses of 10 and 30 mg/kg: the compounds of the invention, administered orally, are capable of restoring the dopaminergic activity inhibited by MPTP in the brain. Such compounds, which cross the blood-brain barrier and possess an effect that promotes communication between neurons, can be used advantageously as an active principle of a medicinal product intended for the treatment of Parkinson's disease.

These in vitro and in vivo results show that the compounds of the invention are capable of modifying the disease mechanisms on certain cellular and animal models and of stopping the degenerative process by generating neuroprotective agents for combating cell death of the dopaminergic neurons. They therefore confirm that these compounds are of interest for use as active principles of medicinal products intended for inhibiting and/or treating neurodegenerative diseases, and more particularly the Parkinson's disease.

The invention also relates to a pharmaceutical composition containing, as an active principle, at least one compound of formula I, or a pharmaceutically acceptable salt thereof.

According to another object, the present application aims at covering the use of said pharmaceutical composition for the inhibition and/or treatment of diseases in which the NURR-1 receptor is involved, notably neurodegenerative diseases, and more particularly the Parkinson's disease.

According to yet another object, the present application aims at covering a method of inhibition and/or treatment of diseases in which the NURR-1 receptor is involved, notably neurodegenerative diseases, and more particularly the Parkinson's disease, which consists of administering, to a patient in need, a therapeutically effective amount of a compound of formula I or of a pharmaceutical composition containing said compound.

These pharmaceutical compositions can be prepared conventionally, by using of pharmaceutically acceptable excipients in order to obtain a form that can be administered by the parenteral route or, preferably, by the oral route, for example tablets or capsules.

In the case of injectable forms, the compounds of formula I will be used advantageously in the form of salts that are soluble in an aqueous medium. As noted previously, the salts are preferably formed between a compound of formula Ib, Id or Ik (acid) and a pharmacologically acceptable non-toxic base. The formulation can be either a solution of the compound in an isotonic aqueous medium in the presence of soluble excipients, or a lyophilizate of the compound, to which the diluent is added extemporaneously. These preparations can be injected in the form of an infusion or as a bolus depending on the patient's needs. In practice, in the case of parenteral administration of the compound, the daily dose in humans will preferably be between 2 and 250 mg.

Preparations for administration by the oral route will preferably be presented in the form of a capsule or a tablet containing the compound of the invention finely ground or preferably micronized, and mixed with excipients known by a person skilled in the art, for example lactose, pregelatinized starch and magnesium stearate. As an example, a mixture constituted of 500 g of the finely ground compound of example 2, 500 g of pregelatinized starch, 1250 g of lactose, 15 g of sodium laurylsulfate and 235 g of polyvinylpyrrolidone was granulated. This granulated mixture was then added to 20 g of magnesium stearate and 80 g of microcrystalline cellulose and the mixture thereby obtained was distributed after grinding and sieving into 260 mg capsules. Capsules were thus obtained, each containing 50 mg of active principle.

In practice, in the case of oral administration of the compound, the daily dose in humans will preferably be between 5 and 500 mg.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A compound selected from the group consisting of:
i) compounds corresponding to formula (I): 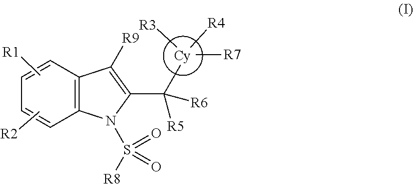
wherein:
Cy represents a phenyl group or a heteroaromatic group having 5 or 6 ring members;
R1 and R2 each independently represent a hydrogen atom, a halogen atom, a nitro group, an alkyl group having 1 to 4 carbon atoms, optionally fully or partially halogenated, an alkoxy group having 1 to 4 carbon atoms, a heterocyclic group having 4 to 6 atoms, a group —SCH₃, OCF₃, —NH₂, —NHR or —NR₂;
R3 and R4 each independently represent a hydrogen atom, a halogen atom, an alkyl group having 1 to 4 carbon atoms, a hydroxyl group or an alkoxy group having 1 to 4 carbon atoms;
R5 and R6 each independently represent a hydrogen atom, a halogen atom, an alkyl group having 1 to 4 carbon atoms, a hydroxyl group; or
R5 and R6 together with the carbon atom to which they are attached form a cycloalkyl group having 3 to 6 carbon atoms, an ethylene group (C═CH₂) or a carbonyl group (C═O);
R7 represents a —COOR group, a bioisosteric group of carboxylic acid or a —CN group;
R8 represents:
an alkyl group having 1 to 6 carbon atoms,
an aryl, heteroaryl, cyclic or heterocyclic group, which group is unsubstituted or substituted with one, two or three substituents which may be identical or different, selected from the group consisting of halogen atoms, alkyl groups having 1 to 6 carbon atoms, optionally fully or partially halogenated or optionally hydroxylated, alkoxy groups having 1 to 6 carbon atoms, optionally fully or partially halogenated, the phenoxy group, cyclic groups having 3 to 6 carbon atoms, aryl and heteroaryl groups, unsubstituted or substituted with one or two substituents, which may be identical or different, selected from halogen atoms and alkyl groups having 1 to 4 carbon atoms, the SCHF₂ and acyl-morpholine groups;
R9 represents a hydrogen atom, a halogen atom or an alkyl group having 1 to 4 carbon atoms;
R represents a hydrogen atom or a linear or branched alkyl group having 1 to 4 carbon atoms; and
ii) pharmaceutically acceptable salts of the compounds of formula (I);
with the proviso that the following compounds are excluded:
2-[[1-(Phenylsulfonyl)-1H-indol-2-yl]carbonyl]-3-pyridinecarboxylic acid;
2-[[5-Methoxy-1-(phenylsulfonyl)-1H-indol-2-yl]carbonyl]-3-pyridine-carboxylic acid;
2-[[6-Methoxy-1-(phenylsulfonyl)-1H-indol-2-yl]carbonyl]-3-pyridine-carboxylic acid;
4-[[1-(Phenylsulfonyl)-1H-indol-2-yl]carbonyl]-3-pyridinecarboxylic acid;
3-[[1-(Phenylsulfonyl)-1H-indol-2-yl]carbonyl]-4-pyridinecarboxylic acid;
4-[[5-Methoxy-1-(phenylsulfonyl)-1H-indol-2-yl]carbonyl]-3-pyridine-carboxylic acid;
2-[[1-(Phenylsulfonyl)-1H-indol-2-yl]carbonyl]-benzoic acid;
3-[[5′-Methoxy-1-(phenylsulfonyl)-1H-indol-2-yl]carbonyl]-4-pyridine-carboxylic acid;
4-[1-Hydroxy-1-[5-methoxy-1-(phenylsulfonyl)-1H-indol-2-yl]ethyl]-3-pyridinecarboxylic acid;
4-[1-[5-Methoxy-1-(phenylsulfonyl)-1H-indol-2-yl]ethyl]-3-pyridinecarboxylic acid;
4-[[3-Chloro-1-(phenylsulfonyl)-1H-indol-2-yl]carbonyl]-3-pyridinecarboxylic acid, methyl ester;
5-[Hydroxy[5-(methylthio)-1-(phenylsulfonyl)-1H-indol-2-yl]methyl]-2-furancarboxylic acid, ethyl ester;
5-[[5-(Methylthio)-1-(phenylsulfonyl)-1H-indol-2-yl]methyl]-2-furancarboxylic acid, ethyl ester;
4-[[3-Bromo-1-(phenylsulfonyl)-1H-indol-2-yl]carbonyl]-3-pyridine-carboxylic acid; and
4-[[1-(Phenylsulfonyl)-1H-inden-2-yl]carbonyl]-benzonitrile.

2. A compound according to claim 1, wherein in formula (I):
Cy represents a group 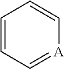
wherein:
A represents a carbon atom monosubstituted with a hydrogen atom or a nitrogen atom, or a heteroaromatic group having 5 ring members and having one or two heteroatoms;
R1 and R2 each independently represent a hydrogen atom, a halogen atom, an alkyl group having 1 to 4 carbon atoms, optionally fully or partially halogenated, an alkoxy group having 1 to 4 carbon atoms, a heterocyclic group having 4 to 6 atoms or a group OCF₃;
R3 and R4 each independently represent a hydrogen atom, a halogen atom, an alkyl group having 1 to 4 carbon atoms, a hydroxyl group or an alkoxy group having 1 to 4 carbon atoms;
R5 and R6 each independently represent a hydrogen atom, a halogen atom, an alkyl group having 1 to 4 carbon atoms, a hydroxyl group; or
R5 and R6 together with the carbon atom to which they are attached form an ethylene group or carbonyl group.
R7 represents a —COOR group, a bioisosteric group of carboxylic acid or a —CN group;
R8 represents:
an alkyl group having 1 to 6 carbon atoms,
an aryl, heteroaryl, cyclic or heterocyclic group, which group is unsubstituted or substituted with one, two or three substituents which may be identical or different, selected from the group consisting of halogen atoms, alkyl groups having 1 to 6 carbon atoms, optionally fully or partially halogenated or optionally hydroxylated, alkoxy groups having 1 to 6 carbon atoms, optionally fully or partially halogenated, the phenoxy group, cyclic groups having 3 to 6 carbon atoms, aryl and heteroaryl groups, unsubstituted or substituted with one or two substituents, which may be identical or different, selected from halogen atoms and alkyl groups having 1 to 4 carbon atoms, the SCHF₂ and acyl-morpholine groups;
R9 represents a hydrogen atom, a halogen atom or an alkyl group having 1 to 4 carbon atoms, and
R represents a hydrogen atom or a linear or branched alkyl group having 1 to 4 carbon atoms.

3. A compound according to claim 1, wherein in formula (I), R8 represents:
an alkyl group having 1 to 6 carbon atoms;
a phenyl group substituted with one or two substituents which may be identical or different, selected from the group consisting of halogen atoms, alkyl groups having 1 to 6 carbon atoms, optionally fully or partially halogenated or optionally hydroxylated, alkoxy groups having 1 to 6 carbon atoms, optionally fully or partially halogenated, the phenoxy group, cyclic groups having 3 to 6 carbon atoms, aryl and heteroaryl groups, unsubstituted or substituted with one or two substituents, which may be identical or different, selected from halogen atoms and alkyl groups having 1 to 4 carbon atoms, the SCHF₂ and acyl-morpholine groups;
a naphthyl group; a thienyl group unsubstituted or substituted with a phenyl group; a pyridinyl group unsubstituted or substituted with a substituent selected from alkoxy groups having 1 to 4 carbon atoms, the phenoxy group, heterocyclic groups having 6 ring members; a benzofuranyl group; a dihydrobenzoxazinone group substituted with a methyl group; or
a tetrahydronaphthyl group, unsubstituted or substituted with one to four alkyl groups having 1 to 4 carbon atoms, a dihydrobenzodioxinyl group unsubstituted or substituted with an alkyl group having 1 to 4 carbon atoms, a dihydrobenzoxazinyl group unsubstituted or substituted with an alkyl group having 1 to 4 carbon atoms, a dihydrobenzodioxepinyl group, a piperidinyl group, a dihydrobenzofuranyl group unsubstituted or substituted with one or two alkyl groups having 1 to 4 carbon atoms, a dihydrobenzopyranyl group unsubstituted or substituted with one or two alkyl groups having 1 to 4 carbon atoms.

4. A compound according to claim 1, wherein in formula (I):
R1 represents a hydrogen atom, a chlorine atom, a bromine atom, a group —CF₃, —OCF₃, —OCH₃—C(CH₃)₃ or pyrrolidinyl; and
R2 represents a hydrogen atom.

5. A compound according to claim 1, wherein in formula (I):
R3 represents hydrogen atom, chlorine, fluorine, a hydroxyl group, a methyl group or a methoxy group; and
R4 represents hydrogen or fluorine.

6. A compound according to claim 1, wherein in formula (I), R8 represents a phenyl group substituted with a C₃-C₄ branched alkyl group.

7. A compound according to claim 1, wherein in formula (I), R9 represents hydrogen, fluorine or a methyl group.

8. A compound according to claim 1, wherein in formula (I):
R5 and R6 each independently represent hydrogen, a methyl group or a hydroxyl group; or
R5 and R6 together with the carbon atom to which they are attached form an ethylene or carbonyl group.

9. A compound according to claim 1, wherein in formula (I), R7 represents an optionally substituted isoxazolone group, an oxadiazolone group, an optionally substituted alkyl sulfonylcarbamoyl group or an optionally substituted aryl sulfonylcarbamoyl group.

10. A compound according to claim 1, wherein R8 represents an aryl, heteroaryl, cyclic or heterocyclic group, which group is unsubstituted or substituted with one, two or three substituents which may be identical or different, selected from the group consisting of phenyl and pyrazolyl, unsubstituted or substituted with one or two substituents, which may be identical or different, selected from halogen atoms and alkyl groups having 1 to 4 carbon atoms.

11. A compound according to claim 1, wherein in formula (I), Cy represents a phenyl, pyridyl, furanyl, thienyl, pyrrolyl or thiazolyl nucleus.

12. A compound according to claim 11, wherein:
R1 represents chlorine, a —CF₃ group or an —OCF₃ group;
R2 represents hydrogen;
R3 represents hydrogen, a halogen, or a methyl group;
R4 represents hydrogen;
R5 and R6 each independently represent hydrogen, a methyl group, or a hydroxyl group; or
R5 and R6 together with the carbon atom to which they are attached form an ethylene or carbonyl group;
R8 represents a phenyl group substituted with a C₃-C₄ branched alkyl group; and
R9 represents hydrogen or a methyl group.

13. A compound according to claim 1, wherein in formula (I):
Cy represents a group 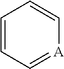
wherein:
A represents a carbon atom monosubstituted with a hydrogen atom or a nitrogen atom,
or a furanyl, thienyl or pyrrolyl group;
R1 represents chlorine, a —CF₃ group or an —OCF₃ group;
R2 represents hydrogen;
R3 represents hydrogen, fluorine, a hydroxyl group, a methyl group or a methoxy group;
R4 represents hydrogen;
R5 and R6 each represent hydrogen;
R8 represents a phenyl group substituted with a C₃-C₄ branched alkyl group, a dihydrobenzodioxinyl group, or a dihydrobenzoxazinyl group unsubstituted or substituted with an alkyl group having 1 to 4 carbon atoms; and
R9 represents a hydrogen atom or a methyl group.

14. A compound according to claim 1, selected from the group consisting of:
4-[[1-[[3-(1-methylethyl)phenyl]sulfonyl]-5-(trifluoromethyl)-1H-indol-2-yl]methyl]benzoic acid,
4-[[1-[[3-(1,1-dimethylethyl)phenyl]sulfonyl]-5-(trifluoromethyl)-1H-indol-2-yl]methyl]benzoic acid,
6-[[1-[[3-(1,1-dimethylethyl)phenyl]sulfonyl]-5-(trifluoromethyl)-1H-indol-2-yl]hydroxymethyl]-3-pyridinecarboxylic acid,
4-[[1-[[3-(1,1-dimethylethyl)phenyl]sulfonyl]-5-trifluoromethyl-1H-indol-2-yl]methyl]-3-fluoro-benzoic acid,
5-[[1-[[3-(1,1-dimethylethyl)-phenyl]sulfonyl]-5-trifluoromethyl-1H-indol-2-yl]methyl]-furan-2-carboxylic acid,
4-[[1-[[3-(1,1-dimethylethyl)-phenyl]sulfonyl]-5-trifluoromethyl-1H-indol-2-yl]methyl]-thiophene-2-carboxylic acid,
5-[[1-[[4-(1-methylethyl)-phenyl]sulfonyl]-5-trifluoromethyl-1H-indol-2-yl]methyl]-thiophene-2-carboxylic acid,
4-[[1-[[4-(1-methylethyl)-phenyl]sulfonyl]-5-trifluoromethyl-1H-indol-2-yl]methyl]-thiophene-2-carboxylic acid,
5-[[1-[[4-methyl-3,4-dihydro-2H-benzo[1,4]oxazin-6-yl]-sulfonyl]-5-trifluoromethyl-1H-indol-2-yl]methyl]-thiophene-2-carboxylic acid,
4-[[1-[(4-methyl-3,4-dihydro-2H-benzo[1,4]oxazin-6-yl)-sulfonyl]-5-trifluoromethyl-1H-indol-2-yl]methyl]-thiophene-2-carboxylic acid,
5-[[1-[(4-methyl-3,4-dihydro-2H-benzo[1,4]oxazin-6-yl)-sulfonyl]-5-trifluoromethyl-1H-indol-2-yl]methyl]-furan-2-carboxylic acid,
5-[[1-[[4-methyl-3,4-dihydro-2H-benzo[1,4]oxazin-6-yl)-sulfonyl]-5-trifluoromethyl-1,4-indol-2-yl]methyl]-furan-3-carboxylic acid,
4-{[1-[[3-(1,1-dimethylethyl)-phenyl]sulfonyl]-5-trifluoromethyl-1H-indol-2-yl]-hydroxymethyl}-1-methyl-1H-pyrrol-2-yl-carboxylic acid (1,1-dimethyl-ethyl)ester,
2-[[1-[[3-(1,1-dimethylethyl)-phenyl]-sulfonyl]-3-methyl-5-trifluoro-methyl-1H-indol-2-yl]methyl]-thiazole-4-carboxylic acid, ethyl ester,
2-[[1-[[3-(1,1-dimethylethyl)-phenyl]sulfonyl]-5-trifluoromethyl-1H-indol-2-yl]methyl]-thiazole-4-carboxylic acid, ethyl ester;
4-[[1-[[3-(1,1-dimethylethyl)phenyl]sulfonyl]-5-(trifluoro)-6-fluoro-1H-indol-2-yl]methyl]benzoic acid, methyl ester,
4-[[1-[[3-(1-methylethyl)phenyl]sulfonyl]-5-(trifluoromethyl)-1H-indol-2-yl]methyl]benzoic acid,
4-[[1-[[3-(1,1-dimethylethyl)phenyl]sulfonyl]-3-fluoro-5-(trifluoromethyl)-1H-indol-2-yl]methyl]-benzoic acid, methyl ester,
4-[[1-[[3-(1,1-dimethylethyl)phenyl]sulfonyl]-3-fluoro-5-(trifluoromethyl)-1H-indol-2-yl]methyl]-benzoic acid,
4-[[[1-[3,3-dimethyl-2,3-dihydro-benzofuran-5-sulfonyl]-5-(chloro)-1H-indol-2-yl]methyl]benzoic acid, methyl ester,
4-[[[1-[3,3-dimethyl-2,3-dihydro-benzofuran-5-sulfonyl]-5-(chloro)-1,4-indol-2-yl]methyl]benzoic acid,
4-[[1-[[3-(1,1-dimethylethyl)phenyl]sulfonyl]-3-methyl-5-(trifluoromethyl)-1H-indol-2-yl]methyl]-benzoic acid,
5-[[1-[[3-(1,1-dimethylethyl)-phenyl]sulfonyl]-5-trifluoromethyl-1H-indol-2-yl]-methyl]-thiophene-2-carboxylic acid, methyl ester,
3-[[1-[[3-(1,1-dimethylethyl)-phenyl]sulfonyl]-5-trifluoromethyl-1H-indol-2-yl]methyl]-4-fluoro-benzoic acid,
3-[[1-[[3-(1,1-dimethylethyl)-phenyl]sulfonyl]-5-trifluoromethyl-1H-indol-2-yl]methyl]-5-fluoro-benzoic acid,
3-[[1-[[3-(1,1-dimethylethyl)-phenyl]sulfonyl]-5-trifluoromethyl-1H-indol-2-yl]methyl]-6-methoxy-benzoic acid,
3-[[1-[[3-(1,1-dimethylethyl)-phenyl]sulfonyl]-5-trifluoromethyl-1H-indol-2-yl]methyl]-4-chloro-6-fluoro-benzoic acid,
3-[[1-[[3-(1,1-dimethylethyl)-phenyl]sulfonyl]-5-trifluoromethyl-1H-indol-2-yl]methyl]-5-pyridine carboxylic acid,
4-[[1-[[3-(1,1-dimethylethyl)-phenyl]sulfonyl]-5-trifluoromethyl-1H-indol-2-yl]methyl]-2-chloro-benzoic acid,
3-[[1-[[3-(1,1-dimethylethyl)-phenyl]sulfonyl]-5-trifluoromethyl-1H-indol-2-yl]methyl]-6-fluoro-benzoic acid,
3-[[1-[[4-methyl-3,4-dihydro-2H-benzo[1,4]oxazin-6-yl]-sulfonyl]-5-trifluoromethyl-1H-indol-2-yl]methyl]-6-fluoro-benzoic acid,
4-[[1-[[3-(1,1-dimethylethyl)phenyl]sulfonyl]-5-(trifluoromethyl)-1H-indol-2-yl]fluoromethyl]benzoic acid,
4-[1-[3-(1,1-dimethylethyl)phenyl]sulfonyl]-5-(trifluoromethyl)-1H-indol-2H-tetrazol-5-yl-benzyl, N-[4-[[1-[[3-(1,1-dimethylethyl)phenyl]sulfonyl]-5-(trifluoromethyl)-1H-indol-2-yl]methyl]benzyl]-methanesulfonamide;
and pharmaceutically acceptable salts of any of the foregoing compounds.

15. A pharmaceutical composition comprising a compound corresponding to formula (I): 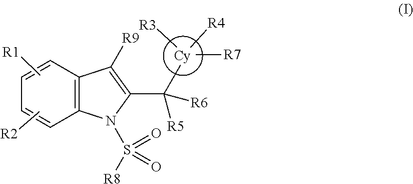
wherein:
Cy represents a phenyl group or a heteroaromatic group having 5 or 6 ring members;
R1 and R2 each independently represent a hydrogen atom, a halogen atom, a nitro group, an alkyl group having 1 to 4 carbon atoms, optionally fully or partially halogenated, an alkoxy group having 1 to 4 carbon atoms, a heterocyclic group having 4 to 6 atoms, a group —SCH₃, OCF₃, —NH₂, —NHR or —NR₂;
R3 and R4 each independently represent a hydrogen atom, a halogen atom, an alkyl group having 1 to 4 carbon atoms, a hydroxyl group or an alkoxy group having 1 to 4 carbon atoms;
R5 and R6 each independently represent a hydrogen atom, a halogen atom, an alkyl group having 1 to 4 carbon atoms, a hydroxyl group; or
R5 and R6 together with the carbon atom to which they are attached form a cycloalkyl group having 3 to 6 carbon atoms, an ethylene group (C═CH₂) or a carbonyl group (C═O);
R7 represents a —COOR group, a bioisosteric group of carboxylic acid or a —CN group;
R8 represents:
an alkyl group having 1 to 6 carbon atoms,
an aryl, heteroaryl, cyclic or heterocyclic group, which group is unsubstituted or substituted with one, two or three substituents which may be identical or different, selected from the group consisting of halogen atoms, alkyl groups having 1 to 6 carbon atoms, optionally fully or partially halogenated or optionally hydroxylated, alkoxy groups having 1 to 6 carbon atoms, optionally fully or partially halogenated, the phenoxy group, cyclic groups having 3 to 6 carbon atoms, aryl and heteroaryl groups, unsubstituted or substituted with one or two substituents, which may be identical or different, selected from halogen atoms and alkyl groups having 1 to 4 carbon atoms, the SCHF₂ and acyl-morpholine groups;
R9 represents a hydrogen atom, a halogen atom or an alkyl group having 1 to 4 carbon atoms; and
R represents a hydrogen atom or a linear or branched alkyl group having 1 to 4 carbon atoms;
or a pharmaceutically acceptable salt thereof as a therapeutically active substance and at least one pharmaceutically acceptable excipient.

16. A method of treating or inhibiting a neurodegenerative disease in a subject in need thereof, said method comprising administering to said subject a therapeutically effective amount of a compound corresponding to formula (I): 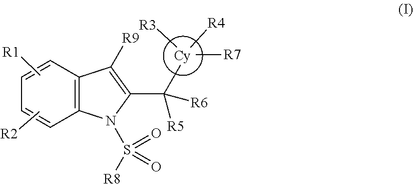
wherein:
Cy represents a phenyl group or a heteroaromatic group having 5 or 6 ring members;
R1 and R2 each independently represent hydrogen, halogen, a nitro group, an alkyl group having 1 to 4 carbon atoms, optionally fully or partially halogenated, an alkoxy group having 1 to 4 carbon atoms, a heterocyclic group having 4 to 6 atoms, a group —SCH₃, OCF₃, —NH₂, —NHR or —NR₂;
R3 and R4 each independently represent hydrogen, a halogen, an alkyl group having 1 to 4 carbon atoms, a hydroxyl group or an alkoxy group having 1 to 4 carbon atoms;
R5 and R6 each independently represent hydrogen, a halogen, an alkyl group having 1 to 4 carbon atoms, or a hydroxyl group; or
R5 and R6 together with the carbon atom to which they are attached form a cycloalkyl group having 3 to 6 carbon atoms, an ethylene group (C═CH₂) or a carbonyl group (C═O);
R7 represents a —COOR group, a bioisosteric group of carboxylic acid or a —CN group;
R8 represents:
an alkyl group having 1 to 6 carbon atoms,
an aryl, heteroaryl, cyclic or heterocyclic group, which group is unsubstituted or substituted with one, two or three substituents which may be identical or different, selected from the group consisting of halogen atoms, alkyl groups having 1 to 6 carbon atoms, optionally fully or partially halogenated or optionally hydroxylated, alkoxy groups having 1 to 6 carbon atoms, optionally fully or partially halogenated, the phenoxy group, cyclic groups having 3 to 6 carbon atoms, aryl and heteroaryl groups, unsubstituted or substituted with one or two substituents, which may be identical or different, selected from halogen atoms and alkyl groups having 1 to 4 carbon atoms, the SCHF₂ and acyl-morpholine groups;
R9 represents a hydrogen atom, a halogen atom or an alkyl group having 1 to 4 carbon atoms; and
R represents a hydrogen atom or a linear or branched alkyl group having 1 to 4 carbon atoms;
or a pharmaceutically acceptable salt thereof.

17. A method according to claim 16, wherein said neurodegenerative disease is Parkinson's disease.

18. A method according to claim 16, wherein in formula (I):
Cy represents a group 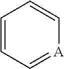
wherein:
A represents a carbon atom monosubstituted with a hydrogen atom or a nitrogen atom, or a heteroaromatic group having 5 ring members and having one or two heteroatoms;
R1 and R2 each independently represent a hydrogen atom, a halogen atom, an alkyl group having 1 to 4 carbon atoms, optionally fully or partially halogenated, an alkoxy group having 1 to 4 carbon atoms, a heterocyclic group having 4 to 6 atoms or a group OCF₃;
R3 and R4 each independently represent a hydrogen atom, a halogen atom, an alkyl group having 1 to 4 carbon atoms, a hydroxyl group or an alkoxy group having 1 to 4 carbon atoms;
R5 and R6 each independently represent a hydrogen atom, a halogen atom, an alkyl group having 1 to 4 carbon atoms, a hydroxyl group; or
R5 and R6 together with the carbon atom to which they are attached form an ethylene group or carbonyl group.
R7 represents a —COOR group, a bioisosteric group of carboxylic acid or a —CN group;
R8 represents:
an alkyl group having 1 to 6 carbon atoms,
an aryl, heteroaryl, cyclic or heterocyclic group, which group is unsubstituted or substituted with one, two or three substituents which may be identical or different, selected from the group consisting of halogen atoms, alkyl groups having 1 to 6 carbon atoms, optionally fully or partially halogenated or optionally hydroxylated, alkoxy groups having 1 to 6 carbon atoms, optionally fully or partially halogenated, the phenoxy group, cyclic groups having 3 to 6 carbon atoms, aryl and heteroaryl groups, unsubstituted or substituted with one or two substituents, which may be identical or different, selected from halogen atoms and alkyl groups having 1 to 4 carbon atoms, the SCHF₂ and acyl-morpholine groups;
R9 represents a hydrogen atom, a halogen atom or an alkyl group having 1 to 4 carbon atoms, and
R represents a hydrogen atom or a linear or branched alkyl group having 1 to 4 carbon atoms.

19. A method according to claim 16, wherein in formula (I), R8 represents:
an alkyl group having 1 to 6 carbon atoms;
a phenyl group substituted with one or two substituents which may be identical or different, selected from the group consisting of halogen atoms, alkyl groups having 1 to 6 carbon atoms, optionally fully or partially halogenated or optionally hydroxylated, alkoxy groups having 1 to 6 carbon atoms, optionally fully or partially halogenated, the phenoxy group, cyclic groups having 3 to 6 carbon atoms, aryl and heteroaryl groups, unsubstituted or substituted with one or two substituents, which may be identical or different, selected from halogen atoms and alkyl groups having 1 to 4 carbon atoms, the SCHF₂ and acyl-morpholine groups;
a naphthyl group; a thienyl group unsubstituted or substituted with a phenyl group; a pyridinyl group unsubstituted or substituted with a substituent selected from alkoxy groups having 1 to 4 carbon atoms, the phenoxy group, heterocyclic groups having 6 ring members; a benzofuranyl group; a dihydrobenzoxazinone group substituted with a methyl group; or
a tetrahydronaphthyl group, unsubstituted or substituted with one to four alkyl groups having 1 to 4 carbon atoms, a dihydrobenzodioxinyl group unsubstituted or substituted with an alkyl group having 1 to 4 carbon atoms, a dihydrobenzoxazinyl group unsubstituted or substituted with an alkyl group having 1 to 4 carbon atoms, a dihydrobenzodioxepinyl group, a piperidinyl group, a dihydrobenzofuranyl group unsubstituted or substituted with one or two alkyl groups having 1 to 4 carbon atoms, a dihydrobenzopyranyl group unsubstituted or substituted with one or two alkyl groups having 1 to 4 carbon atoms.

20. A method according to claim 16, wherein said compound is selected from the group consisting of:
4-[[1-[[3-(1-methylethyl)phenyl]sulfonyl]-5-(trifluoromethyl)-1H-indol-2-yl]methyl]benzoic acid,
4-[[1-[[3-(1,1-dimethylethyl)phenyl]sulfonyl]-5-(trifluoromethyl)-1H-indol-2-yl]methyl]benzoic acid,
6-[[1-[[3-(1,1-dimethylethyl)phenyl]sulfonyl]-5-(trifluoromethyl)-1H-indol-2-yl]hydroxymethyl]-3-pyridinecarboxylic acid,
4-[[1-[[3-(1,1-dimethylethyl)phenyl]sulfonyl]-5-trifluoromethyl-1H-indol-2-yl]methyl]-3-fluoro-benzoic acid,
5-[[1-[[3-(1,1-dimethylethyl)-phenyl]sulfonyl]-5-trifluoromethyl-1H-indol-2-yl]methyl]-furan-2-carboxylic acid,
4-[[1-[[3-(1,1-dimethylethyl)-phenyl]sulfonyl]-5-trifluoromethyl-1H-indol-2-yl]methyl]-thiophene-2-carboxylic acid,
5-[[1-[[4-(1-methylethyl)-phenyl]sulfonyl]-5-trifluoromethyl-1H-indol-2-yl]methyl]-thiophene-2-carboxylic acid,
4-[[1-[[4-(1-methylethyl)-phenyl]sulfonyl]-5-trifluoromethyl-1H-indol-2-yl]methyl]-thiophene-2-carboxylic acid,
5-[[1-[[4-methyl-3,4-dihydro-2H-benzo[1,4]oxazin-6-yl]-sulfonyl]-5-trifluoromethyl-1H-indol-2-yl]methyl]-thiophene-2-carboxylic acid,
4-[[1-[(4-methyl-3,4-dihydro-2H-benzo[1,4]oxazin-6-yl)-sulfonyl]-5-trifluoromethyl-1H-indol-2-yl]methyl]-thiophene-2-carboxylic acid,
5-[[1-[(4-methyl-3,4-dihydro-2H-benzo[1,4]oxazin-6-yl)-sulfonyl]-5-trifluoromethyl-1H-indol-2-yl]methyl]-furan-2-carboxylic acid,
5-[[1-[[4-methyl-3,4-dihydro-2H-benzo[1,4]oxazin-6-yl)-sulfonyl]-5-trifluoromethyl-1H-indol-2-yl]methyl]-furan-3-carboxylic acid,
4-{[1-[[3-(1,1-dimethylethyl)-phenyl]sulfonyl]-5-trifluoromethyl-1H-indol-2-yl]-hydroxymethyl}-1-methyl-1H-pyrrol-2-yl-carboxylic acid (1,1-dimethyl-ethyl)ester,
2-[[1-[[3-(1,1-dimethylethyl)-phenyl]-sulfonyl]-3-methyl-5-trifluoro-methyl-1H-indol-2-yl]methyl]-thiazole-4-carboxylic acid, ethyl ester,
2-[[1-[[3-(1,1-dimethylethyl)-phenyl]sulfonyl]-5-trifluoromethyl-1H-indol-2-yl]methyl]-thiazole-4-carboxylic acid, ethyl ester;
4-[[1-[[3-(1,1-dimethylethyl)phenyl]sulfonyl]-5-(trifluoro)-6-fluoro-1H-indol-2-yl]methyl]benzoic acid, methyl ester,
4-[[1-[[3-(1-methylethyl)phenyl]sulfonyl]-5-(trifluoromethyl)-1H-indol-2-yl]methyl]benzoic acid,
4-[[1-[[3-(1,1-dimethylethyl)phenyl]sulfonyl]-3-fluoro-5-(trifluoromethyl)-1H-indol-2-yl]methyl]-benzoic acid, methyl ester,
4-[[1-[[3-(1,1-dimethylethyl)phenyl]sulfonyl]-3-fluoro-5-(trifluoromethyl)-1H-indol-2-yl]methyl]-benzoic acid,
4-[[[1-[3,3-dimethyl-2,3-dihydro-benzofuran-5-sulfonyl]-5-(chloro)-1H-indol-2-yl]methyl]benzoic acid, methyl ester,
4-[[[1-[3,3-dimethyl-2,3-dihydro-benzofuran-5-sulfonyl]-5-(chloro)-1H-indol-2-yl]methyl]benzoic acid,
4-[[1-[[3-(1,1-dimethylethyl)phenyl]sulfonyl]-3-methyl-5-(trifluoromethyl)-1H-indol-2-yl]methyl]-benzoic acid,
5-[[1-[[3-(1,1-dimethylethyl)-phenyl]sulfonyl]-5-trifluoromethyl-1H-indol-2-yl]-methyl]-thiophene-2-carboxylic acid, methyl ester,
3-[[1-[[3-(1,1-dimethylethyl)-phenyl]sulfonyl]-5-trifluoromethyl-1H-indol-2-yl]methyl]-4-fluoro-benzoic acid,
3-[[1-[[3-(1,1-dimethylethyl)-phenyl]sulfonyl]-5-trifluoromethyl-1H-indol-2-yl]methyl]-5-fluoro-benzoic acid,
3-[[1-[[3-(1,1-dimethylethyl)-phenyl]sulfonyl]-5-trifluoromethyl-1H-indol-2-yl]methyl]-6-methoxy-benzoic acid,
3-[[1-[[3-(1,1-dimethylethyl)-phenyl]sulfonyl]-5-trifluoromethyl-1H-indol-2-yl]methyl]-4-chloro-6-fluoro-benzoic acid,
3-[[1-[[3-(1,1-dimethylethyl)-phenyl]sulfonyl]-5-trifluoro methyl-1H-indol-2-yl]methyl]-5-pyridine carboxylic acid,
4-[[1-[[3-(1,1-dimethylethyl)-phenyl]sulfonyl]-5-trifluoro methyl-1H-indol-2-yl]methyl]-2-chloro-benzoic acid,
3-[[1-[[3-(1,1-dimethylethyl)-phenyl]sulfonyl]-5-trifluoromethyl-1H-indol-2-yl]methyl]-6-fluoro-benzoic acid,
3-[[1-[[4-methyl-3,4-dihydro-2H-benzo[1,4]oxazin-6-yl]-sulfonyl]-5-trifluoromethyl-1H-indol-2-yl]methyl]-6-fluoro-benzoic acid,
4-[[1-[[3-(1,1-dimethylethyl)phenyl]sulfonyl]-5-(trifluoromethyl)-1H-indol-2-yl]fluoromethyl]benzoic acid,
4-[1-[3-(1,1-dimethylethyl)phenyl]sulfonyl]-5-(trifluoromethyl)-1H-indol-2H-tetrazol-5-yl-benzyl,
N-[4-[[1-[[3-(1,1-dimethylethyl)phenyl]sulfonyl]-5-(trifluoromethyl)-1H-indol-2-yl]methyl]benzyl]-methanesulfonamide;
and pharmaceutically acceptable salts of any of the foregoing compounds.
